(19) United States
(12) Patent Application Publication
(10) Pub. No.: US 2009/0018136 A1
(43) Pub. Date: Jan. 15, 2009

(54) METHODS AND COMPOSITIONS FOR THE TREATMENT OF VASCULAR DISEASE

(76) Inventors: Daniel I. Oppenheimer, Castro Valley, CA (US); Emil D. Kakkis, Novato, CA (US); Alejandro Dorenbaum, Mill Valley, CA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 12/134,023

(22) Filed: Jun. 5, 2008

Related U.S. Application Data
(63) Continuation-in-part of application No. PCT/US2006/046449, filed on Dec. 5, 2006.
(60) Provisional application No. 60/742,578, filed on Dec. 5, 2005.
(60) Provisional application No. 60/764,979, filed on Feb. 3, 2006.
(60) Provisional application No. 60/817,847, filed on Jun. 30, 2006.

(51) Int. Cl. A61K 31/4985; A61K 31/53; A61P 7/00

(52) U.S. Cl. 514/243; 514/249

(57) ABSTRACT

The present invention is directed to a novel methods and compositions for the therapeutic intervention of vascular complications associated with diabetes, hyperlipidemias, and various cardiovascular disorders including but not limited to recalcitrant hypertension, coronary artery disease, pulmonary arterial hypertension, congestive heart failure, and hemolytic anemias. More specifically, the specification describes methods and compositions for treating such vascular disorders using compositions comprising BH4 and derivative thereof. Combination therapies of BH4 and other therapeutic regimens are contemplated.

### CROSS REFERENCE TO RELATED APPLICATIONS

This is a continuation-in-part of international application PCT/US2006/046449, filed Dec. 5, 2006, which claims priority of U.S. Provisional Application No. 60/742,578 filed Dec. 5, 2005, U.S. Provisional Application No. 60/764,979 filed Feb. 3, 2006, and U.S. Provisional Application No. 60/817,847 filed Jun. 30, 2006, each of which is hereby incorporated by reference in its entirety.

### BACKGROUND

1. Field of the Invention

The present invention is generally directed to the therapeutic intervention of vascular disease. More particularly, the present invention is directed to methods and compositions for the treatment of endothelial dysfunction associated with vascular dysfunction.

2. Background of the Related Art

Diabetes and its cardiovascular complications are the leading cause of mortality and morbidity in the United States and the western world. Several causative factors are implicated in the development of these diseases including hereditary predisposition to the disease, gender, lifestyle factors such as smoking and diet, age, insulin resistance, hypertension, and hyperlipidemia, including hypercholesterolemia.

Treatment options for diabetes and related cardiovascular diseases include various therapeutic agents such as cholesterol lowering drugs (e.g. statins), vasoactive agents (e.g. s. PPAR gamma ligands, β blockers), ACE inhibitors, Angiotensin II receptor blockers, calcium channel blockers, vitamins and antioxidants (e.g. niacin, ascorbic acid or vitamin C). The rationale for using statin drugs to lower plasma cholesterol fails to explain why coronary heart attacks generally occur in individuals with non-critical blockages and why blockages do not occur in capillaries or veins. When used, statin drugs reduce the risk of a recurrent coronary event, only by 30 to 40%. The rationale for vasoactive drugs is to reduce blood pressure by acting directly or indirectly on vascular and/or cardiac smooth muscle, thereby decreasing vascular resistance to flow. However, such drugs do not treat the initial cause of elevated pressure and abnormal flow, but seek to reduce the resulting effect of the disorder. Such drugs activate the sympathetic nervous system by way of a baroreceptor reflex to produce an increased heart rate and force of myocardial contraction, which are not beneficial or desirable effects. Vitamin E, vitamin C, probucol and β-carotene constitute most of antioxidants currently applied for treatment of diabetes. Unfortunately, however, none of these agents when administered alone or in combination with other agents can adequately address cellular (i.e., skin or endothelial) dysfunction and other oxidative stress-mediated pathologies. Because of their ‘mode’ of action, tissue uptake and other relevant characteristics, all currently available antioxidants can only indirectly affect endothelium derived relaxing factor metabolism and action act only on certain reactive oxygen species (ROS). Further, they may adversely affect the course of the disease if incorrectly dosed.

Moreover, present treatments for such disorders are short-term and have serious shortcomings with respect to long-term effectiveness. The use of therapeutic drugs for diabetes and the related acute and chronic occlusive vascular diseases of the heart central and peripheral vascular systems have to date been ineffective for favorable long-term results and do not treat the underlying pathophysiology or restore the structure and function of the blood vessels to normal states.

Each of the therapeutic agents while having some beneficial effects on the patient have serious side effects and often need to be taken in high non-physiological doses. The side effects are often dose related. The adverse effects for the classes of therapeutic agents above include hypoglycemia, renal dysfunction, and myopathy including rhadomyolysis, hepatotoxicity, airway resistance and teratogenic effects if taken by pregnant subjects. Other side effects for such drugs include headache, heart palpitations, anxiety, mild depression, myocardial infarction, congestive heart failure, fatigue and weakness. Further, a pharmacological dose may not be specific in its effect on the initial molecular cause of the disease activity, and treats a limited spectrum of effects in the diseases, which are dependent on several factors. In some cases, the adverse effects may be as simple as flushing and dyspepsia but result in a serious lack of patient compliance with the treatment regimen. To offset the adverse effects of the drugs, various combination therapies have been suggested as treatment options.

Thus, there remains a need for a consistently effective and specific agent for the management of endothelial dysfunction underlying vascular disease without causing severe adverse side effects. The present invention is directed to addressing such a need.

### SUMMARY

In general, the invention describes a therapeutic intervention of endothelial dysfunction resulting in vascular disease. The invention contemplates methods and compositions for treating a subject having a disease or disorder characterized by endothelial dysfunction, comprising administering to said subject a composition comprising tetrahydrobiopterin (BH4) or a precursor or derivative thereof, alone or in combination with a therapeutic agent, wherein said administration is effective in alleviating endothelial dysfunction of said subject as compared to said endothelial dysfunction in the absence of said BH4-containing composition. The invention further contemplates a method of treating a subject with endothelial dysfunction comprising administering a factor or combination of factors that enhances the production of the vasodilator nitric oxide (NO) alone or in combination with a therapeutic agent.

In one aspect, the invention provides a method for treating a subject diagnosed as having diabetes-related vascular complications comprising administering BH4 or a precursor or derivative thereof, alone or in combination with another agent, wherein such agent is a therapeutic agent or a factor that enhances the production of the vasodilator nitric oxide (NO).

In one embodiment, diabetes-related vascular complications include but are not limited to disorders of general vascular functions such as abnormal vascular compliance, endothelial dysfunction and hypertension; recalcitrant hypertension and disorders of insulin sensitivity and glucose control. In another embodiment diabetes-related vascular complications include but are not limited to abnormal peripheral perfusion such as intermittent claudication, reduced peripheral perfusion, decreased skin blood flow and defective wound healing. In a further embodiment, diabetes-related vascular complications include but are not limited to cardiac disease such as congestive heart failure, pulmonary hypertension with or without congestive heart failure, exercise-associated angina, coronary artery disease and related atherosclerosis, albuminuria associated with coronary artery disease; ophthalmic disease such as optic atrophy and diabetic retinal disease; and renal disease such as microalbuminuria in diabetic renal disease, renal failure and decreased glomerular filtration rate.

In a related embodiment, the therapeutic agent is an agent used to treat diabetes, including but not limited to agents that improve insulin sensitivity such as PPAR gamma ligands (thiazolidinedones, glitazones, troglitazones, rosiglitazone (Avandia), pioglitazone), stimulators of insulin secretion such as sulphonylureas (gliquidone, tolbutamide, glimepride, chlorpropamide, glipizide, glyburide, acetohexamide) and meglitinides (meglitinide, repaglinide, nateglinide) and agents that reduce liver production of glucose such as metformin.

In a second aspect, the invention provides a method for treating a subject diagnosed as having vascular disease unrelated to diabetes comprising administering BH4 or a precursor or derivative thereof alone or in combination with another agent, wherein such agent is a therapeutic agent or a factor that enhances the production of the vasodilator nitric oxide (NO). Such vascular disease unrelated to diabetes is selected from the group consisting of pulmonary vascular disease, hemolytic anemias, stroke and related ischemic vascular disease (such as stroke, cardiac or coronary disease, arteriosclerosis, peripheral vascular disease, peripheral arterial disease, intermittent claudication), thrombosis, transplant-related endothelial dysfunction, cardiac or coronary artery disease, albuminuria associated with chronic kidney disease, hypertension and isolated systolic hypertension. In one embodiment, pulmonary vascular disease includes but is not limited to pulmonary hypertension in sickle cell anemia and other hemoglobinopathies, idiopathic pulmonary hypertension, pulmonary arterial hypertension, persistent pulmonary hypertension of the newborn (PPHN). In a further embodiment, hemolytic anemias include hereditary hemolytic anemias and acquired hemolytic anemia. Hereditary hemolytic anemias include but are not limited to sickle-cell anemia, thalassemia, hemolytic anemia due to G6PD deficiency, pyruvate kinase deficiency, hereditary elliptocytosis, hereditary spherocytosis, hereditary stomatocytosis, hereditary ovalocytosis, paroxysmal nocturnal hemoglobinuria, and hemoglobin SC disease. Acquired hemolytic anemias include but are not limited to microangiopathic hemolytic anemia, idiopathic autoimmune hemolytic anemia, non-immune hemolytic anemia caused by chemical or physical agents or devices (left ventricular assist devices), mechanical heart valves and bypass devices), and secondary immune hemolytic anemia.

In another embodiment, stroke and related ischemic vascular disease includes but is not limited to vasospasm, such as post-stroke cerebrovascular spasm. Thrombosis includes but is not limited to thrombogenesis, thrombosis, clotting, and coagulation. In a further embodiment, transplant-related endothelial dysfunction includes but is not limited to vascular dysfunction after solid organ transplantation and cyclosporine A induced endothelial dysfunction. In yet another embodiment, cardiac or coronary disease includes but is not limited to congestive heart failure, vascular dysfunction and angina associated with hypercholesterolemia, and vascular dysfunction and angina associated with tobacco smoking.

In a related embodiment the therapeutic agent is an agent used to treat vascular disease, including but not limited to endothelin receptor antagonists commonly used for the treatment of hypertension and other endothelial dysfunction-related disorders, such as bosentan, darusentan, enrasentan, tezosentan, atrasentan, ambrisentan sitaxsentan; smooth muscle relaxants such as PDE5 inhibitors (indirect-acting) and minoxidil (direct-acting); angiotensin converting enzyme (ACE) inhibitors such as captopril, enalapril, lisinopril, fosinopril, perindopril, quinapril, trandolapril, benazepril, ramipril; angiotensin II receptor blockers such as irbesartan, losartan, valsartan, eprosartan, olmesartan, candesartan, telmisartan; beta blockers such as atenolol, metoprolol, nadolol, bisoprolol, pindolol, acebutolol, betaxolol, propranolol; diuretics such as hydrochlorothiazide, furosemide, torsemide, metolazone; calcium channel blockers such as amlodipine, felodipine, nisoldipine, nifedipine, verapamil, diltiazem; alpha receptor blockers doxazosin, terazosin, alfuzosin, tamsulosin; and central alpha agonists such as clonidine.

In one embodiment, the combination of BH4 or a precursor or derivative of BH4 with a PDE5 inhibitor provides unexpectedly beneficial effects on vascular pressure parameter(s). Thus the BH4 or precursor or derivative is expected to attenuate adverse effects of such drugs, e.g. attenuate elevation of blood pressure.

In a related aspect, the invention provides methods of treating hypertension in humans by administering to a human suffering from hypertension an amount of purified 6R BH4 at a dose of at least 200 mg daily (e.g. given as 100 mg twice daily), or at least 150 mg daily, or at least 100 mg daily. In such exemplary embodiments the range of doses of BH4 may also be less than 500 mg daily, or less than 400 mg daily or less than 300 mg daily. In one embodiment, the BH4 is administered at a daily dose of at least 5 mg/kg/day, or at least 10 mg/kg/day, up to 20 mg/kg/day or 30 mg/kg/day. The BH4 may be administered alone or in combination with other therapeutic agents used to treat vascular disease or hyperlipidemia, such as any of those listed above or below.

In a third aspect, the invention provides a method for treating a subject diagnosed as having hyperlipidemia comprising administering BH4 or a precursor or derivative thereof alone or in combination with another agent, wherein such agent is a therapeutic agent or a factor that enhances the production of the vasodilator nitric oxide (NO). In a related embodiment the therapeutic agent is an agent used to treat hyperlipidemia, including but not limited to agents that lower LDL such as statins (atorvastatin, fluvastatin, lovastatin, pravastatin, rosuvastatin calcium, simvastatin) and nicotinic acid, cholesteryl ester transfer protein inhibitors (such as torcetrapib), agents that stimulate PPAR alpha such as fibrates, gemfibrozil, fenofibrate, bezafibrate, ciprofibrate, agents that bind and prevent readsorption of bile acids and reduce cholesterol levels such as bile acid sequestrants, cholestyramine and colestipol, and cholesterol absorption inhibitors. BH4 or a precursor or derivative may be administered with combinations of such agents (such as statin/cholesteryl ester transfer protein inhibitor combinations) and is expected to attenuate adverse effects relating to elevation of blood pressure.

In a fourth aspect, the invention further contemplates a method of treating a subject with endothelial dysfunction comprising administering a factor or combination of factors that enhances or normalizes the production of the vasodilator nitric oxide (NO) alone or in combination with a therapeutic agent. In one embodiment, such factor(s) enhances the activity or expression the de novo biosynthesis of BH4 and is selected from the group consisting of guanosine triphosphate cyclohydrolase I (GTPCH1), 6-pyruvoyltetrahydropterin synthase (PTPS) and sepiapterin reductase. In a preferred embodiment of the invention, BH4 synthesis is increased by increasing the expression of GTPCH1 expression by the use of any one or more cyclic adenosine monophosphate (cAMP) analogs or agonists including forskolin, 8-bromo cAMP or other agents that function to increase cAMP mediated cell signaling, for example, cytokines and growth factors including interleukin-1, interferon-gamma (IFN-γ), tumor necrosis factor alpha (TNF-α), c-reactive protein, HMG-CoA-reductases (statins like atorvastatin) nerve growth factor (NGF), epidermal growth factor (EGF), hormones including adrenomedullin and estradiol benzoate, and other compounds such as NADPH and NADPH analogs, caffeine, cyclosporine A methyl-xanthines including 3-isobutyl-1-methyl xanthine, theophylline, reserpine, hydrogen peroxide.

One embodiment of invention therefore relates to increasing GTPCH1 levels by inhibiting the degradation of 3′5′-cyclic nucleotides using inhibitors of the eleven phosphodiesterases families (PDE1-11) including PDE1, PDE3, PDE5. The PDE inhibitors of the present invention include Viagra/sildanefil, cialis/tadalafil, vardenafil/levitra, udenafil, 8-Methoxymethyl-IBMX, UK-90234, dexamethasone, hesperetin, hesperedins, Irsogladine, vinpocetine, cilostamide, rolipram, ethyl beta-carboline-3-carboxylate (beta-CCE), tetrahydro-beta-carboline derivatives, 3-O-methylquercetin and the like.

Another embodiment of the invention relates to increasing the levels of BH4 by increasing the levels of BH4-synthesizing enzymes by gene therapy or endothelium-targeted delivery of polynucleotides of the synthetic machinery of BH4. Yet another embodiment of the invention relates to increasing the levels of BH4 by supplementation with BH4-synthesizing enzymes GTPCH1, PTPS, SR, PCD, DHPR and DHFR. It is contemplated that BH4-synthesizing enzymes encompasses all natural and unnatural forms of the enzymes including mutants of the proteins.

Another embodiment of the invention relates to increasing BH4 levels by diverting the substrate 7,8-dihydroneopterin triphosphate towards BH4 synthesizing enzyme PTPS instead of alkaline phosphatase (AP) by inhibiting AP activity. The agents or compounds that inhibit the activity of AP include phosphate analogs, levamisole, and L-Phe. Another embodiment of the invention relates to agents or compounds that inhibit alkaline phosphatase includes the small inhibitory RNA (siRNA), antisense RNA, dsDNA, small molecules, neutralizing antibodies, single chain, chimeric, humanized and antibody fragments to inhibit the synthesis of alkaline phosphatase.

Another embodiment of the invention includes agents or compounds that enhance the activity of catalysts or cofactors needed for the synthesis of enzymes of the de novo synthesis pathway of BH4 synthesis.

Another embodiment of the invention includes agents or compounds that prevent the degradation of the enzymes needed for the synthesis of BH4. Yet another embodiment of the invention includes agents or compounds that prevent the degradation of the catalysts needed for the synthesis of BH4 and its synthetic enzymes including GTPCH1, PTPS and SR.

Another embodiment of the invention relates to increasing the levels of BH4 by increasing the reduction of BH2 via the salvage pathway. In vivo, BH4 becomes oxidized to BH2. BH2 which exist as the quinoid form (qBH2) and as the 7,8-dihydropterin which is reduced to BH4 by DHPR and DHFR respectively. A preferred embodiment of the invention relates to increasing the regeneration or salvage of BH4 from BH2 by modulating the activity and synthesis of the enzymes PCD, DHPR and DHFR using agents or compounds that pathway NADPH, thiols, perchloromercuribenzoate, hydrogen peroxide and the like.

Another embodiment of the invention relates to agents that stabilize BH4 by decreasing the oxidation of BH4 using agents or compounds such as antioxidants including ascorbic acid (vitamin C), alpha tocopherol (vitamin E), tocopherols (e.g vitamin A), selenium, beta-carotenes, carotenoids, flavones, flavonoids, folates, flavones, flavanones, isoflavones, catechins, anthocyanidins, and chalcones.

In a further embodiment, such factor(s) may increase the activity or expression of nitric oxide synthase and thereby enhance the generation of NO.

In yet another embodiment, the invention contemplates factors that inhibit the GTPCH feedback regulatory protein, GFRP. A preferred embodiment of the invention relates to agents or compounds that inhibit the binding of BH4 to the GTPCH1/GFRP complex, thereby preventing the feedback inhibition by BH4. Agents or compounds of this invention include competitive inhibitors such as alternate forms of BH4 with altered affinities for the complex, structural analogs etc. Still another embodiment of the invention includes agents or compounds that enhance the binding of L-phenylalanine to CTPCH1/GFRP inducing the synthesis of BH4. Another embodiment of the invention includes agents or compounds that increase the levels of L-Phe such as precursors of L-Phenylalanie, which serves to inhibit the feedback inhibition of GTPCH1 by GFRP and BH4.

Yet another embodiment of the invention relates to agents or compounds that modulate the activity or the synthesis of GFRP. A preferred embodiment of the invention includes agents or compounds that inhibit the activity of GFRP. Another embodiment of the invention includes the use of siRNA, small molecules, antibodies, antibody fragments and the like to inhibit the synthesis of GFRP.

The invention further contemplates agents or compounds that are the precursors of BH4 including guanosine tripohosphate, 7,8-dihydro-neopterin triphosphate and 6-pyrovoyl tetrahydropbiopterin.

BH4 is administered in an amount of between about 0.1 mg/kg to about 30 mg/kg, for example, between about 5 mg/kg and 10 mg/kg daily. BH4 may be administered in a single daily dose or in multiple doses on a daily basis. In some embodiments, the BH4 therapy is not continuous, but rather BH4 is administered on a daily basis until improvement in clinical endpoints (e.g. normal blood pressure in patients with recalcitrant hypertension) is maintained.

In yet another aspect of the invention, the administration of a BH4 derivative, is carried out at an unexpectedly lower dose that still achieves therapeutic efficacy. Such BH4 derivatives are contemplated to have improved biological properties relative to natural BH4. In one embodiment, it is contemplated that the efficacious doses of BH4 derivatives for hypertension, vascular disease, or any of the diseases described herein will be lower than the usual dose of BH4 for treatment of other BH4-responsive disorders such as hyperphenylalanemia. In particular, the invention contemplates that any of the 1′,2′-diacyl-(6R,S)-5,6,7,8-tetrahydro-L-biopterins or lipoidal tetrahydrobiopterins described herein exhibit improved biological properties at low doses.

Also contemplated is a composition comprising a stabilized, crystallized form of BH4 that is stable at room temperature for more than 8 hours and a pharmaceutically acceptable carrier, diluent or excipient. Preferably, the BH4 being administered is a stabilized crystallized form of BH4 that has greater stability than non-crystallized stabilized BH4. More preferably, the stabilized crystallized form of BH4 comprises at least 99.5%, or 99.9% pure 6R BH4. Precursors such as dihydrobiopterin (BH2), and sepiapterin also may be administered. BH4 may be administered orally.

BH4 may be administered intramuscularly, subcutaneously, or intravenously, via intrapulmonary administration either alone or in combination with other therapeutic agents or interventions currently used to treat endothelial dysfunction including but not limited to agents and intervention used to maintain homeostasis, adjuvant therapy and specific therapy. Specific therapy may include an agent used to treat diabetes, including but not limited to agents that improve insulin sensitivity such as PPAR gamma ligands (thiazolidinedones, glitazones, troglitazones, rosiglitazone (Avandia), pioglitazone), stimulators of insulin secretion such as sulphonylureas (gliquidone, tolbutamide, glimepride, chlorpropamide, glipizide, glyburide, acetohexamide) and meglitinides (meglitinide, repaglinide, nateglinide) and agents that reduce liver production of glucose such as metformin. Specific therapy may include an agent used to treat vascular disease, including but not limited to endothelin receptor antagonists commonly used for the treatment of hypertension and other endothelial dysfunction-related disorders, such as bosentan, darusentan, enrasentan, tezosentan, atrasentan, ambrisentan sitaxsentan; smooth muscle relaxants such as PDE5 inhibitors (indirect-acting) and minoxidil (direct-acting); angiotensin converting enzyme (ACE) inhibitors such as captopril, enalapril, lisinopril, fosinopril, perindopril, quinapril, trandolapril, benazepril, ramipril; angiotensin II receptor blockers such as irbesartan, losartan, valsartan, eprosartan, olmesartan, candesartan, telmisartan; beta blockers such as atenolol, metoprolol, nadolol, bisoprolol, pindolol, acebutolol, betaxolol, propranolol; diuretics such as hydrochlorothiazide, furosemide, torsemide, metolazone; calcium channel blockers such as amlodipine, felodipine, nisoldipine, nifedipine, verapamil, diltiazem; alpha receptor blockers doxazosin, terazosin, alfuzosin, tamsulosin; and central alpha agonists such as clonidine. Specific therapy may include an agent used to treat hyperlipidemia, including but not limited to agents that lower LDL such as statins (atovastatin, fluvastatin, lovastatin, pravastatin, rosuvastatin calcium, simvastatin) and nicotinic acid, agents that stimulate PPAR alpha such as fibrates, gemfibrozil, fenofibrate, bezafibrate, ciprofibrate, agents that bind and prevent readsorption of bile acids and reduce cholesterol levels such as bile acid sequestrants, cholestyramine and colestipol, and cholesterol absorption inhibitors. Agents used to maintain homeostatic levels of BH4 and/or NO production may include factor(s) that enhance the activity or expression the de novo biosynthesis of BH4, such as guanosine triphosphate cyclohydrolase I (GTPCH1), 6-pyruvoyltetrahydropterin synthase (PTPS) and sepiapterin reductase; factor(s) that may act to stabilize BH4, such as Vitamin C, ascorbic acid, alpha tocopherol; factor(s) that increase the activity or expression of nitric oxide synthase and thereby enhance the generation of NO; and factors that inhibit the GTPCH feedback regulatory protein, GFRP.

The present invention contemplates administering one or more of crystal form of BH4 selected from the group consisting of crystal polymorph form A, crystal polymorph form B, crystal polymorph form F, crystal polymorph form J, crystal polymorph form K, crystal hydrate form C, crystal hydrate form D, crystal hydrate form E, crystal hydrate form H, crystal hydrate form O, solvate crystal form G, solvate crystal form I, solvate crystal form L, solvate crystal form M, solvate crystal form N, and combinations thereof.

In other embodiments, BH4 may be administered optionally and concurrently with folates, including folate precursors, folic acids, and folate derivatives. Such folates include but are not limited to tetrahydrofolate is 5-formyl-(6S)-tetrahydrofolic acid and salts thereof, 5-methyl-(6S)-tetrahydrofolic acid and salts thereof, 5,10-methylene-(6R)-tetrahydrofolic acid and salts thereof, 5,10-methenyl-(6R)-tetrahydrofolic acid and salts thereof, 10-formyl-(6R)-tetrahydrofolic acid, 5-formimino-(6S)-tetrahydrofolic acid salts thereof, (6S)-tetrahydrofolic acid and salts thereof, and combinations of the foregoing. In a further embodiment, BH4 may be administered optionally and concurrently with arginine.

Each of the uses, compositions, and methods of treatment described herein is contemplated to optionally exclude patients having one or more of the following conditions: subjects suffering from defects in BH4 synthesis, BH4 deficiency, PKU, HPA, autism, Parkinson's disease, spinocerebellar degeneration, Machado-Joseph disease, muscular dystrophy, Sjögren's syndrome, and depression.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1. Production of NO

FIG. 2. Endothelial Dysfunction and BH4 Deficiency

FIG. 3. Pathophysiology of Secondary BH4 Deficiency

FIG. 4. Secondary BH4 deficiency and Uncoupled eNOS

FIG. 5. Diabetes and BH4 Deficiency

FIG. 6. Characteristic X-ray diffraction pattern exhibited by (6R)-BH4 form B.

FIG. 7. Characteristic X-ray diffraction pattern exhibited by (6R)-BH4 form A.

FIG. 8. Characteristic X-ray diffraction pattern exhibited by (6R)-BH4 form F.

FIG. 9. Characteristic X-ray diffraction pattern exhibited by (6R)-BH4 form J.

FIG. 10. Characteristic X-ray diffraction pattern exhibited by (6R)-BH4 form K.

FIG. 11. Characteristic X-ray diffraction pattern exhibited by (6R)-BH4 form C.

FIG. 12. Characteristic X-ray diffraction pattern exhibited by (6R)-BH4 form D.

FIG. 13. Characteristic X-ray diffraction pattern exhibited by (6R)-BH4 form E.

FIG. 14. Characteristic X-ray diffraction pattern exhibited by (6R)-BH4 form H.

FIG. 15. Characteristic X-ray diffraction pattern exhibited by (6R)-BH4 form O.

FIG. 16. Characteristic X-ray diffraction pattern exhibited by (6R)-BH4 form G.

FIG. 17. Characteristic X-ray diffraction pattern exhibited by (6R)-BH4 form I.

FIG. 18. Characteristic X-ray diffraction pattern exhibited by (6R)-BH4 form L.

FIG. 19. Characteristic X-ray diffraction pattern exhibited by (6R)-BH4 form M.

FIG. 20. Characteristic X-ray diffraction pattern exhibited by (6R)-BH4 form N.

FIG. 21. Blood pressure response of humans administered BH4 at a dose of 5 mg/kg/day

FIG. 22. Blood pressure response of humans administered BH4 at a dose of 200 mg twice daily.

FIG. 23. Blood pressure response of humans administered BH4 at a dose of 100 mg twice daily.

FIG. 24. Mean systolic pressure after treatment with BH4 and sildenafil, alone or in combination, over the 24 hour period after dosing.

FIG. 25. Mean diastolic pressure after treatment with BH4 and sildenafil, alone or in combination, over the 24 hour period after dosing.

FIG. 26. Mean arterial pressure after treatment with BH4 and sildenafil, alone or in combination, over the 24 hour period after dosing.

FIG. 27. Mean arterial pulse pressure after treatment with BH4 and sildenafil, alone or in combination, over the 24 hour period after dosing.

FIG. 28. Mean (+dP/dtₘₐₓ) after treatment with BH4 and sildenafil, alone or in combination, over the 24 hour period after dosing.

FIG. 29. Mean heart rate after treatment with BH4 and sildenafil, alone or in combination, over the 24 hour period after dosing.

FIG. 30. Flowchart for the randomized, placebo-controlled study of two doses of oral 6R-BH4 on vascular function in subjects with coronary artery disease.

### DESCRIPTION OF THE PREFERRED EMBODIMENTS

### I. Role of BH4 in Endothelial Dysfunction

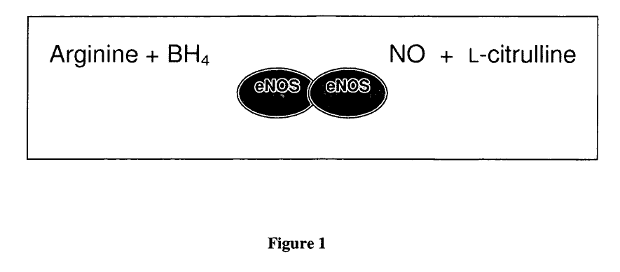

BH4 is a required cofactor in the biosynthesis of NO from arginine by the enzyme endothelial nitric oxide synthase (eNOS) as shown in FIG. 1. The inability to generate NO from arginine and BH4 is localized to the endothelium and is therefore called endothelial dysfunction.

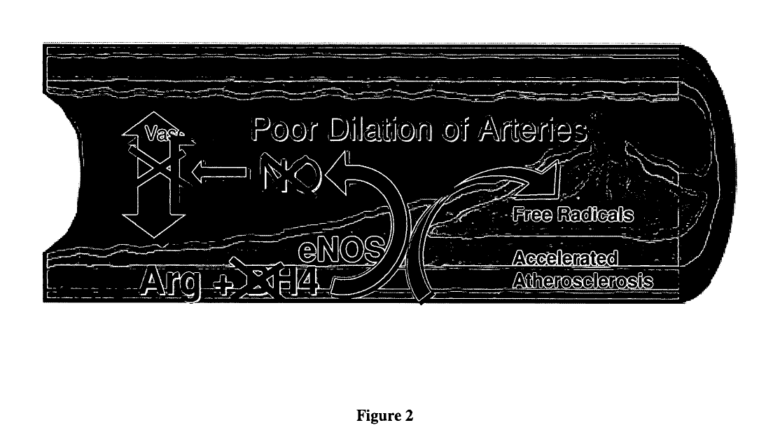

Endothelial dysfunction is characterized by the abnormal inability of the endothelium (the single cell layer lining that forms the barrier between blood vessel walls and the blood) to produce the vasodilator NO using endothelial eNOS, as shown in FIG. 2 (Alp, et al., J Clin Invest 2003; 112(5):725-735; Katusic, Am J Physiol Heart Circ Physiol 2001; 281(3):H981-H986; Meininger, et al., Biochem J 2000; 349(Pt 1):353-356; Pieper, J Cardiovasc Pharmacol 1997; 29(1):8-15; Fukuda, et al., Heart 2002; 87(3):264-269; Ueda, et al., J Am Coll Cardiol 2000; 35(1):71-75; Maier, et al., J Cardiovasc Pharmacol 2000; 35(2):173-178; Kakoki, et al., J Am Soc Nephrol 2000; 11(2):301-309; de Vriese, et al., Br J Pharmacol 2000; 130(5):963-974; Pannirselvam, et al., Br J Pharmacol 2002; 136(2):255-263; Shinozaki, et al., Circ Res 2000; 87(7):566-573; Cosentino, et al., Eur Heart J 1998; 19 Suppl G:G3-G8).

This deficiency of NO results in excessive vasoconstriction as a direct effect, and as secondary effects, there are increased free radicals generated (Channon, Trends Cardiovasc Med 2004; 14(8):323-327; Meininger, et al., Biochem J 2000; 349(Pt 1):353-356; Berka, et al., Biochemistry 2004; 43(41):13137-13148; Vasquez-Vivar, et al, Biochem J 2002; 362(Pt 3):733-739; Cosentino, et al., Cardiovasc Res 1999; 43(2):274-278; Guzik, et al., Circulation 2002; 105(14):1656-1662), accelerated vascular injury and atherosclerosis (Cosentino, et al., Eur Heart J 1998; 19 Suppl G:G3-G8; Vasquez-Vivar, et al., Biochem J 2002; 362(Pt 3):733-739) and increased thrombogenicity and coagulation. The overall data show that NO supports blood flow, and in its absence, there is reduced blood flow and increased potential for atherosclerosis and clotting. Thus, endothelial dysfunction is associated with vasoconstriction/hypertension, inadequate dilation, accelerated atherosclerosis, increased thrombogenesis, increased selections and a higher cardiac event rate.

Studies using transgenic mouse models of sickle cell disease have consistently shown that tissue NOS levels and basal NOS activity are increased whereas vasodilatory responses to endothelium-dependent agonists such as acetylcholine were impaired (Reiter, et al., Current Opinions in Hematology 10:99-107 (2003)). Thus, findings from animal studies suggest that NO plays a compensatory role in response to chronic vascular injury associated with sickle cell disease. However, in human studies, other factors appear to influence the bioavailability of NO. In women with sickle cell disease, ovarian-produced estrogens protect endothelial function by enhancing NOS expression and basal endothelial NO production. Thus women with sickle cell disease had normal basal and stimulated NO production. However, men with sickle cell disease and all patients with vaso-occlusive crisis and acute chest syndrome had reduced basal and stimulated NO production. The combined effects of circulating plasma hemoglobin and superoxide result in the destruction of NO. In sickle cell disease, an increase in NO production reduces the endothelial expression of adhesion molecules and subsequent adhesion of red blood cells and leukocytes, thereby preventing the development of a vaso-occlusive crises (Space, et al., Am. J. Hematology 63:200-204 (2000)). NO also reduces platelet activation and thrombin generation, thereby preventing coagulation. Other beneficial actions of NO in the management of sickle cell disease include inactivation of reactive oxygen species (ROS) and the relaxation of smooth muscle in subjects with asthma and sickle cell disease.

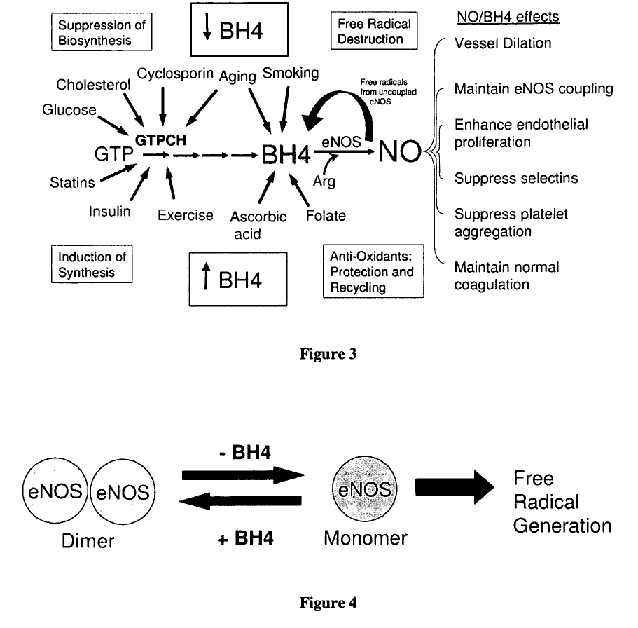

The basis for involvement of BH4 in endothelial dysfunction has become clear in the last few years and appears through various mechanisms to be due to secondary BH4 deficiency as the key pathophysiologic step as shown in Table 1 (Meininger, et al., Biochem J 2000; 349(Pt 1):353-356; Meininger, et al., FASEB J 2004). BH4 is a required on a mole for mole basis as a cofactor in the enzyme reaction of eNOS to make NO from arginine (FIG. 1) and in many cases BH4 deficiency caused by various factors has been shown to be the underlying cause of the inability to make NO. FIG. 3 provides a diagrammatic representation of the pathophysiology of secondary BH4 deficiency.

TABLE 1
Diseases and Mechanisms Behind BH4 Deficiency

| | | BH4 replacement data or |
|---|---|---|
| General Population | Pathophysiology | pathophysiological support |
| Diabetes | Glucose suppresses BH4 | Replacement reverses vascular |
| | biosynthesis through GTPCH | disease in arm, eye and kidney |
| Coronary artery | Atherosclerotic changes are | Addition of BH4 to vascular |
| disease | associated with decreasing | specimens from patients |
| | endothelial function | normalizes the dilatory response |
| | | to acetyl choline |
| Hypercholesterolemia | Increased cholesterol suppresses | Replacement of BH4 restores |
| | BH4 biosynthesis at GTPCH | vascular function |
| Smoking | Oxidation products of smoke | Replacement restores vascular |
| | deplete endothelium of BH4 | dilation in smokers |
| | pool | |
| Idiopathic Pulmonary | Mutations in BH4 biosynthesis | BH4 biosynthesis knockout |
| hypertension | decrease endothelial function | mouse has PH and is reversed |
| | | with BH4 |
| Stroke | Endothelial injury post stroke | Infusion with nitrite reverses |
| | leads to vasospasm | vasospasm |
| Hemolytic anemia | Free heme creates free radicals | No data yet, but destruction of |
| | that destroy endothelial function | BH4 pool is likely mechanism |
| Transplant patients | Ischemia and Cyclosporin A | BH4 replacement reduces |
| | induce vascular dysfunction by | vascular injury in rat cardiac |
| | repression of BH4 biosynthesis | transplant model |
| Pulmonary | Infants, many of diabetic | NO inhalation improves |
| hypertension in the | mothers, have decreased | oxygenation and reduces |
| newborn (PPHN) | endothelial function | pulmonary hypertension |

As a secondary function, recent data show that BH4 is also required to stabilize the normal functioning, coupled, dimeric form of eNOS (FIG. 4). When BH4 is deficient, eNOS dissociates into dysfunctional monomers and is uncoupled leading to the generation of free radical species such as peroxynitrite that then lead to further destruction of the BH4 pool exacerbating the underlying deficiency (Channon, Trends Cardiovasc Med 2004; 14(8):323-327; Werner, et al., Exp Biol Med (Maywood) 2003; 228(11):1291-1302; Vasquez-Vivar, et al., Biochem J 2002; 362(Pt 3):733-739; Wei, Chem Rev 2003; 103(6):2365-2383).

In diabetics, the higher glucose levels directly suppress BH4 biosynthesis by decreasing the expression of the first enzyme GTP cyclohydrolase (GTPCH) (Meininger, et al., Biochem J 2000; 349(Pt 1):353-356; Meininger, et al., FASEB J 2004; Cai, et al., Cardiovasc Res 2005; 65(4):823-831) and leads to a vascular dysfunction that is responsive to BH4 replacement in animals (Alp, et al., J Clin Invest 2003; 112(5):725-735; Yu, et al., J Ocul Pharmacol Ther 2001; 17(2):123-129; Meininger, et al., Biochem J 2000; 349(Pt 1):353-356; Pieper, J Cardiovasc Pharmacol 1997; 29(1):8-15; Pannirselvam. et al., Br J Pharmacol 2002; 136(2):255-263; Hamon, et al., Clin Chim Acta 1989; 181(3):249-253) and humans (Nystrom, et al., Am J Physiol Endocrinol Metab 2004; Channon, Trends Cardiovasc Med 2004; 14(8):323-327; Werner, et al., Exp Biol Med (Maywood) 2003; 228(11):1291-1302; Heitzer, et al., Diabetologia 2000; 43(11):1435-1438; Guzik, et al., Circulation 2002; 105(14):1656-1662). Excess cholesterol in hypercholesterolemia also appears to repress GTPCH, which is reversed by BH4 administration (Fukuda, et al., Heart 2002; 87(3):264-269). Patients with heart failure and coronary artery disease, also appear to have endothelial dysfunction due to BH4 deficiency (Setoguchi, et al., J Cardiovasc Pharmacol 2002; 39(3):363-368; Maier, et al., J Cardiovasc Pharmacol 2000; 35(2):173-178). In smoking, the excess oxidizing compounds from smoke destroy the BH4 pool and function is restored by BH4 replacement (Katusic, Am J Physiol Heart Circ Physiol 2001; 281(3):H981-H986; Ueda, et al., J Am Coll Cardiol 2000; 35(1):71-75). In hemolytic anemias, the free heme generates free radicals which may also deplete the BH4 pool (Rother, et al., JAMA 2005; 293(13):1653-1662). The resulting BH4 deficiency leads to the inability to produce NO. In subjects with sickle cell disease, BH4 replacement would be effective in the management of pulmonary arterial hypertension, ischemia-reperfusion injury, and organ damage due to poor vascular flow, vaso-occlusive crises due to blockage and/or adhesion by red blood cells and white blood cells, and coagulation.

The list of possible indications for the use of 6R-BH4 based on the recent literature includes diabetes (Nystrom, et al., Am J Physiol Endocrinol Metab 2004; Channon, Trends Cardiovasc Med 2004; 14(8):323-327; Werner, et al., Exp Biol Med (Maywood) 2003; 228(11):1291-1302; Alp, et al., J Clin Invest 2003; 112(5):725-735; Katusic, Am J Physiol Heart Circ Physiol 2001; 281(3):H981-H986; Yu, et al., J Ocul Pharmacol Ther 2001; 17(2):123-129; Heitzer, et al., Diabetologia 2000 43(11):1435-1438; Meininger, et al., Biochem J 2000; 349(Pt 1):353-356; Pieper, et al., J Cardiovasc Pharmacol 1997; 29(1):8-15), hypercholesterolemia (Fukuda, et al., Heart 2002; 87(3):264-269), smoking (Lowe, et al., Drug Metab Dispos 2004; Ueda, et al., J Am Coll Cardiol 2000; 35(1):71-75), congestive heart failure (Setoguchi, et al., J Cardiovasc Pharmacol 2002; 39(3):363-368), atherosclerosis (Channon, Trends Cardiovasc Med 2004; 14(8):323-327; Katusic, Am J Physiol Heart Circ Physiol 2001; 281(3):H981-H986; Maier, et al., J Cardiovasc Pharmacol 2000; 35(2):173-178), pulmonary hypertension (Pritchard, et al., Circulation 2005; 111(16):2022-2024; Khoo, et al., Circulation 2005; 111(16):2126-2133), coronary artery disease (Nystrom, et al., Am J Physiol Endocrinol Metab 2004; Maier, et al., J Cardiovasc Pharmacol 2000; 35(2):173-178; Setoguchi, et al., J Am Coll Cardiol 2001; 38(2):493-498), post-organ transplant reperfusion injury/vascular dysfunction (Yamashiro, et al., J Cardiovasc Surg (Torino) 2003; 44(1):37-49; Kakoki, et al., J Am Soc Nephrol 2000; 11(2):301-309; Duranski et al., J Clin Invest 2005), post-stroke vasospasm (Pluta, et al., JAMA 2005; 293(12):1477-1484), and hemolytic anemias, including sickle cell disease (U.S. Patent Application Publication No. 2003/0078231; Wood et al., Free Radical Biology & Medicine 40:1443-1453 (2006)). In all of these indications, BH4 deficiency occurs and leads to a deficiency of nitric oxide (NO) production and the inability of the vasculature to respond to normal dilatory signals.

In general, the invention describes a therapeutic intervention of endothelial dysfunction resulting in vascular disease. The invention contemplates methods and compositions for treating a subject having a disease or disorder characterized by endothelial dysfunction, comprising administering to said subject a composition comprising tetrahydrobiopterin (BH4) or a precursor or derivative thereof, alone or in combination with a therapeutic agent, wherein said administration is effective in alleviating endothelial dysfunction of said subject as compared to said endothelial dysfunction in the absence of said BH4-containing composition. The invention further contemplates a method of treating a subject with endothelial dysfunction comprising administering a factor or combination of factors that enhances the production of the vasodilator nitric oxide (NO) alone or in combination with a therapeutic agent.

### 1. Diabetes-Related Vascular Complications

Diabetes mellitus and other cardiovascular disease states are characterized by loss of nitric oxide (NO) bioactivity resulting in altered the balance between vasodilators and vasoconstrictors in the endothelium and contributing to endothelial dysfunction. Endothelial dysfunction underlies the increased vasoconstriction resulting in hypertension, inadequate dilation response to flow or other signals, increased thrombogenesis and platelet aggregation, increased cell surface adhesion molecules such as the selectins, increased coagulation factors and accelerated atherosclerosis due to excess free radical production such as reactive oxygen species (ROS), for e.g., superoxide molecules. Since NO plays a central role in maintaining vascular homeostasis, loss of NO bioactivity contributes to vascular disease pathogenesis and is a marker of adverse outcome of the diseases.

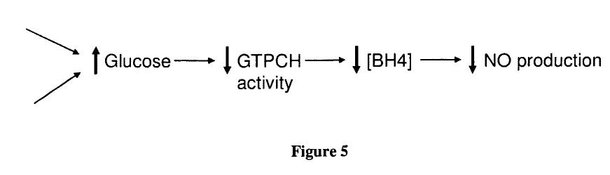

Recent findings suggest that accelerated catabolism of BH4 in arteries exposed to oxidative stress contributes to the pathogenesis of the endothelial dysfunction known to exist in the arteries of diabetics. Additionally, elevated glucose prevents an increase in cellular levels of BH4. Deficiency of ascorbic acid as observed in diabetics also contributes to reduced availability of BH4 levels to eNOS. Fortunately, in animals and humans, experimental supplementation of BH4 has demonstrated beneficial effects on endothelial function. High-concentration BH4 supplementation studies using vessel rings from animals with diabetes or atherosclerosis and in mammary artery rings from patients with diabetes support the idea that BH4 could potentially ameliorate endothelial dysfunction and restore vascular function. Some examples of the positive effects on BH4 on cardiovascular and diabetic subjects include: BH4 administration appears to augment NO-mediated effects on forearm blood flow in patients with diabetes or hypercholesterolemia but not normal subjects (Heitzer et al, Diabetologia. 43(11): 1435-8 (2000)). Acute BH4 restores vascular function in venous grafts and arteries in diabetic subjects undergoing coronary artery bypass graft surgery (Guzik et al, Circulation 105(14):1656-1662 (2002)). BH4 increases insulin sensitivity in patients with Type II diabetes and coronary heart disease compared to control subjects (Nystrom et al, Am J Physiol Endocrinol Metab. 2004 November; 287(5):E919-25. Epub (2004)). Supplementation of BH4 precursors in the biosynthetic pathway has also been shown to assist in increased BH4 levels intracellularly and improve NO synthesis in vivo and improve endothelial function. FIG. 5 shows role of BH4 deficiency in diabetes.

### 2. Uncontrolled Hypertension

The effect of BH4 was studied in humans with hypertension. Eight patients with poorly controlled hypertension (systolic BP>135 mmHg) and on treatment with traditional antihypertensive therapy were given oral BH4 (5 mg/kg/d, 10 mg/kg/d) over a period of 8 weeks. Patients were assessed for brachial artery flow-mediated vasodilatation (FMD) and dilations after sublingual nitroglycerin at baseline and after 8 weeks of treatment and 1 week post-treatment, and blood pressure changes weekly for 9 weeks and 6 weeks post-treatment. FMD increased significantly after 8 weeks of BH4 treatment and returned to baseline after 1 week following withdrawal. Oral BH4 significantly reduced systolic and diastolic pressures and blood pressure returned to baseline values 6 weeks after cessation of therapy. Blood pressure reduction was significant after 3 weeks of treatment and both doses of BH4 produced similar effects. The findings show that BH4 improves endothelium-dependent vasodilation in patients with poorly controlled hypertension (Lefever, et al., American Heart Association Scientific Sessions 2003, Session No. AOP.43.3, Presentation Number 2378)

### 3. Coronary Artery Disease

Coronary artery disease may be characterized as combination of endothelial dysfunction and accelerated atherosclerosis. BH4 has been shown to restore vessel function in multiple studies. BH4 increased coronary vessel flow (Fukuda, et al., 2002). BH4 infused during angiography prevented abnormal vasoconstriction to acetylcholine in patients with endothelial dysfunction based on changes in coronary blood flow velocity (Maier et al (2000) J. Cardiovasc Pharmacol 35:173-178). BH4 therapy provided the combination of improved coronary blood flow and reduced atherosclerosis/thrombogenicity. Mortality from repeat MI in diabetics is very high.

### 4. Pulmonary Vascular Disease—Pulmonary Arterial Hypertension

There is some evidence that NO deficiency exists in pulmonary arterial hypertension (PAH): (1) Patients with PAH were shown to have decreased breath NO levels. (2) Knock-out mice with defects in BH4 biosynthesis and BH4 deficiency develop pulmonary hypertension as their major manifestation (Nandi et al 2005, Circulation 111:2086). (3) Recent data on sildenafil (PDE5 inhibitor) suggests that NO signaling is deficient in PAH. However, there is no human BH4 replacement data found so far.

5. Hemolytic Anemias, including Sickle Cell Disease

Some data exists that show that endothelial dysfunction occurs in patients with hemolytic anemias and lack of NO underlies the problem. BH4 deficiency is likely cause due to oxidative destruction of BH4 pool. Animal studies suggest that NO plays a compensatory role in response to chronic vascular injury associated with sickle cell disease. The combined effects of circulating plasma hemoglobin and superoxide result in the destruction of NO (Reiter, et al., Current Opinions in Hematology 10:99-107 (2003)). New therapeutic approaches that increase the bioavailability of NO or counteract the oxidative stress and uncontrolled free radical proliferation associated with sickle cell disease have been considered. The co-administration of arginine with hydroxyurea may augment the production of NO and improve use of arginine in patients with SCD at steady state (Morris, et al., J. Pediatric Hematology 25(8):629-634 (2003)). In addition to hydroxyurea and arginine, other therapies such as inhaled NO to increase NO levels, allopurinol to reduce NO destruction, and statins and sildenafil to amplify the NO response have been considered (Mack, et al., Intl. J. Biochem. Cell Biol., In Press (2006)). U.S. Patent Application Publication 2003/0078231 describes the use of the orthomolecular sulpho-adenosylmethionine derivatives as a nutritional or food supplement with antioxidant properties to treat several diseases resulting from oxidative stress and uncontrolled free radical proliferation, including sickle cell anemia. US Patent Application Publication No. 2005/0239807 A1 describes the use of an inhibitor of reactive oxygen generating enzyme which includes a group providing NO donor bioactivity (e.g. allopurinol) to treat diseases associated with oxidative stress such as sickle cell anemia.

In sickle cell disease, NO reduces the endothelial expression of adhesion molecules and subsequent adhesion of red blood cells and leukocytes, thereby preventing the development of a vaso-occlusive crises (Space, et al., Am. J. Hematology 63:200-204 (2000)). The cell-associated NADPH oxidase was shown to be a source of superoxide (Wood, et al. FASEB J. 19(8):989-991 (2005)). The rapid generation of superoxide radicals associated with Sickle Cell Disease may trigger the production of secondary reactive oxygen and nitrogen metabolites such as OH and ONOO which are known to oxidize BH4, thereby causing a deficiency in BH4. In one study, the administration of sepiapterin, a precursor of BH4, to sickle cell transgenic (PS) mice was associated with an attenuation of blood cell adhesion (Wood, et al., J. Free Radical Biology & Medicine 40:1443-1453 (2006)). Although consistent with the present invention, the authors specifically indicate that sepiapterin lacks the anti- and auto oxidative properties of exogenous BH4, the use of which is contemplated in the present invention. Further, as described above, transgenic sickle cell mouse models may not accurately reflect the complex homeostatic mechanisms that control the levels of NOS, NO and BH4 observed in humans (Reiter, et al., Current Opinion in Hematology 10:99-107 (2003)).

### 6. Intermittent Claudication

Intermittent claudication is the most prominent symptom of peripheral artery disease. It is most often caused by atherosclerotic narrowing of the iliac and femoral arteries, often combined with lesions in distal arteries of the leg. In intermittent claudication, blood flow is sufficient for the needs of a person at rest. However, when such person exercises, the vessels become blocked, thereby limiting blood flow and reducing the oxygen supply to levels insufficient to meet the exercising muscles' demands. In response, the body reduces the release of factors, such as NO that would dilate the blood vessels and increases the release of factors that constrict the blood vessels, such as thromboxane, serotonin, angiotensin II, endothelin, norepinephrine. There is also evidence that blood cells may become abnormal and prone to forming clots. Symptoms of intermittent claudication include withered calf muscles, hair loss over the toes and feet, thick toenails, shiny tight skin, painful ulcers in the toe that are discolored black and do not bleed, and in some cases, blood clot formation in the arteries of the legs. Intermittent claudication may affect as many as 5% of men over 50 years. For 10-20% affected, the symptoms worsen and may result in amputation in one in twenty due to development of a gangrenous limb. Therapy for intermittent claudication includes lifestyle changes (exercise, cessation of smoking and alcohol consumption), exercise therapy, medication (pentoxifyline, nafronyl, antithrombotics, phosphodiesterase inhibitor cilostazol) and supplements (Vitamin E, B, and C), a diet low in cholesterol and endovascular repair (Hiatt, Atherosclerosis Supplements (2005) in press; Wolosker, et al., Clinics 60(3):193-200 (2005)). Studies in diabetics show BH4 administration was associated with improved ischemia-induced blood flow and FMD.

### 7. Persistent Pulmonary Hypertension of the Newborn

Term babies with Persistent Pulmonary Hypertension of the Newborn (PPHN) have a high mortality rate and number perhaps 40,000 babies per year. The mortality rate is very high, perhaps in the 20-50% range. Many of these babies are LGA and infants of diabetic mothers, consistent with the relationship of BH4 deficiency due to hyperglycemia.

### 8. Stroke and Related Ischemic Vascular Disease

Data from a canine stroke model shows that post-stroke vasospasm around the site of the clot, causes extension and greater damage than the original event and can be prevented by infusing nitrite solutions.

### II. BH4 and the Treatment of Vascular Disorders

The present invention describes a pharmaceutical intervention of vascular disorders based on the administration of BH4. It is further contemplated that any type of BH4, in a stabilized or other form may be used to treat that patient population comprising subjects with various forms of vascular disease, including but not limited to recalcitrant or uncontrolled hypertension, isolated systolic hypertension, peripheral arterial disease, intermittent claudication, coronary artery disease, pulmonary arterial hypertension, and hemolytic anemias including sickle cell disease, albuminuria associated with chronic kidney disease in the presence and absence of diabetes. Such BH4-based compositions may be administered alone or in combination with any other therapeutic agent and/or intervention that is commonly used for the treatment of vascular disorders. Such agents include but are not limited to agents used to treat diabetes, including but not limited to agents that improve insulin sensitivity such as PPAR gamma ligands (thiazolidinedones, glitazones, troglitazones, rosiglitazone (Avandia), pioglitazone), stimulators of insulin secretion such as sulphonylureas (gliquidone, tolbutamide, glimepride, chlorpropamide, glipizide, glyburide, acetohexamide) and meglitinides (meglitinide, repaglinide, nateglinide) and agents that reduce liver production of glucose such as metformin. Such agents include but are not limited to agents used to treat vascular disease, including but not limited to endothelin receptor antagonists commonly used for the treatment of hypertension and other endothelial dysfunction-related disorders, such as bosentan, darusentan, enrasentan, tezosentan, atrasentan, ambrisentan sitaxsentan; smooth muscle relaxants such as PDE5 inhibitors (indirect-acting) and minoxidil (direct-acting); angiotensin converting enzyme (ACE) inhibitors such as captopril, enalapril, lisinopril, fosinopril, perindopril, quinapril, trandolapril, benazepril, ramipril; angiotensin II receptor blockers such as irbesartan, losartan, valsartan, eprosartan, olmesartan, candesartan, telmisartan; beta blockers such as atenolol, metoprolol, nadolol, bisoprolol, pindolol, acebutolol, betaxolol, propranolol; diuretics such as hydrochlorothiazide, furosemide, torsemide, metolazone; calcium channel blockers such as amlodipine, felodipine, nisoldipine, nifedipine, verapamil, diltiazem; alpha receptor blockers doxazosin, terazosin, alfuzosin, tamsulosin; and central alpha agonists such as clonidine. Such agents include but are not limited to agents used to treat hyperlipidemia, including but not limited to agents that lower LDL such as statins (atovastatin, fluvastatin, lovastatin, pravastatin, rosuvastatin calcium, simvastatin) and nicotinic acid, agents that stimulate PPAR alpha such as fibrates, gemfibrozil, fenofibrate, bezafibrate, ciprofibrate, agents that bind and prevent readsorption of bile acids and reduce cholesterol levels such as bile acid sequestrants, cholestyramine and colestipol, and cholesterol absorption inhibitors.

Certain embodiments of the present invention are directed to treating vascular dysfunction administering to the subject a composition comprising BH4 or a precursor or derivative thereof alone or in combinations with conventional vascular treatment, wherein the administration of BH4 alone or in combination with conventional vascular therapy is effective to improve clinically relevant endpoints of said subject as compared to said concentration in the absence of BH4 alone or in combination with conventional vascular therapy.

One embodiment of the invention entails administering a BH4 composition to any individual with abnormal endpoints in an amount effective to normalize values. In a preferred embodiment, such individual is diagnosed with the specific vascular disease. The invention contemplates administering the stabilized BH4 compositions described herein to patients diagnosed with a specific vascular disease characterized by specific symptoms and/or common tests used to diagnose a specific vascular disease in an amount effective to improve endpoints to normal levels.

Those of skill in the art would understand that the invention contemplates treating patients having typical symptoms with BH4 to produce normal values for clinically relevant endpoints. Further, any changes in endpoint values within the minimal of normal ranges for clinically relevant endpoints will be considered a therapeutic outcome for the therapeutic regimens for the patients.

### 1. Combination Therapy

The present invention further contemplates the therapeutic intervention of various types of vascular dysfunction by administration of BH4 alone or in combination with an agent or intervention commonly used to treat vascular dysfunction. It should be understood that the BH4 therapies may be combined with conventional agents or interventions to treat vascular dysfunction to effect the therapeutic increase in clinically relevant endpoints for such disease in such patients. As described above, treatment of vascular dysfunction is directed at maintaining homeostasis, providing adjuvant therapy and providing specific therapy to improve clinical relevant endpoints. Homeostasis is maintained by correcting factors that predispose to vascular dysfunction including levels of BH4 and NO production without increasing the generation of damaging free radicals. Adjuvant therapy consists of administering agents or interventions that increase the effectiveness of the primary therapy. Specific therapy is directed at maintaining normal clinical relevant endpoints. The conventional agents and interventions currently used to treat vascular dysfunction have been discussed above. Some of the conventional interventions used to manage or treat vascular dysfunction include anti-diabetic agents, agents used to treat various types of vascular disease, and anti-hyperlipidemic therapy. Agents used to treat diabetes include but are not limited to agents that improve insulin sensitivity such as PPAR gamma ligands (thiazolidinedones, glitazones, troglitazones, rosiglitazone (Avandia), pioglitazone), stimulators of insulin secretion such as sulphonylureas (gliquidone, tolbutamide, glimepride, chlorpropamide, glipizide, glyburide, acetohexamide) and meglitinides (meglitinide, repaglinide, nateglinide) and agents that reduce liver production of glucose such as metformin. Agents used to treat vascular disease include but are not limited to endothelin receptor antagonists commonly used for the treatment of hypertension and other endothelial dysfunction-related disorders, such as bosentan, darusentan, enrasentan, tezosentan, atrasentan, ambrisentan sitaxsentan; smooth muscle relaxants such as PDE5 inhibitors (indirect-acting) and minoxidil (direct-acting); angiotensin converting enzyme (ACE) inhibitors such as captopril, enalapril, lisinopril, fosinopril, perindopril, quinapril, trandolapril, benazepril, ramipril; angiotensin II receptor blockers such as irbesartan, losartan, valsartan, eprosartan, olmesartan, candesartan, telmisartan; beta blockers such as atenolol, metoprolol, nadolol, bisoprolol, pindolol, acebutolol, betaxolol, propranolol; diuretics such as hydrochlorothiazide, furosemide, torsemide, metolazone; calcium channel blockers such as amlodipine, felodipine, nisoldipine, nifedipine, verapamil, diltiazem; alpha receptor blockers doxazosin, terazosin, alfuzosin, tamsulosin; and central alpha agonists such as clonidine. Agents used to treat hyperlipidemia include but are not limited to agents that lower LDL such as statins (atovastatin, fluvastatin, lovastatin, pravastatin, rosuvastatin calcium, simvastatin) and nicotinic acid, agents that stimulate PPAR alpha such as fibrates, gemfibrozil, fenofibrate, bezafibrate, ciprofibrate, agents that bind and prevent readsorption of bile acids and reduce cholesterol levels such as bile acid sequestrants, cholestyramine and colestipol, and cholesterol absorption inhibitors.

The BH4 to be administered alone or in combination with therapeutic agents and interventions to manage and/or treat vascular dysfunction, need not necessarily be a stabilized BH4 composition described herein. Those of skill in the art are aware of methods of producing a BH4 composition that is unstable at room temperature and in light. While therapies using such a composition are hindered by the instability of the BH4 composition, its use is still contemplated in certain combination therapies where patients suffering from vascular dysfunction are treated with a course of BH4 treatment and conventional therapy used to treat vascular disease.

The methods and compositions for producing such a stabilized BH4 compositions are described in further detail herein. The stabilized BH4 compositions of the present invention comprise BH4 crystals that are stable at room temperature for longer than 8 hours, or at least 24 hours, 3 months, 6 months, 9 months, 12 months or longer. The methods and compositions of the present invention contemplate pharmaceutical compositions of the stabilized BH4 alone that may be delivered through any conventional route of administration, including but not limited to oral, intramuscular injection, subcutaneous injection, intravenous injection and the like. The compositions of the present invention may further comprise BH4 compositions in combination with an antioxidant that aids in prolonging the stability of the BH4 composition.

Certain methods of the invention involve the combined use of BH4 and conventional agents and interventions to effect a therapeutic outcome in patients with vascular disease. To achieve the appropriate therapeutic outcome in the combination therapies contemplated herein, one would generally administer to the subject the BH4 composition and the agents/intervention in a combined amount effective to produce the desired therapeutic outcome. This process may involve administering the BH4 composition and the agent/intervention at the same time. This may be achieved by administering a single composition or pharmacological formulation that includes both the therapeutic agent and BH4 in a combined dosage form or administering the BH4 formulation at the same time as the interventions is being conducted. Alternatively, the agent/intervention is taken at about the same time as a pharmacological formulation (tablet, injection or drink) of BH4. In other alternatives, the BH4 treatment may precede or follow the agent/intervention by intervals ranging from minutes to hours. In embodiments where the agent/intervention and the BH4 compositions are administered separately, one would generally ensure that both agents are exerting their effect concurrently, such that the BH4 will still be able to exert an advantageous effect on the patient. In such instances, it is contemplated that one would administer the BH4 within about 2-6 hours (before or after) of the agent/intervention, with a delay time of only about 1 hour being most preferred. However, it should be understood the 2-6 hour time frame between administration of the two agents is merely exemplary, it may be that longer time intervals, e.g., 24 hours, 36 hours, 48 hours, 72 hours, one week or more between administration of the BH4 and the second agent/intervention also is contemplated. In certain embodiments, it is contemplated that the BH4 therapy will be a continuous therapy where a daily dose of BH4 is administered to the patient indefinitely.

### 2. BH4 Compositions for Use in Therapy

The present section provides a discussion of the compositions that may be used in the treatments contemplated herein.

U.S. Pat. Nos. 5,698,408; 2,601,215; 3,505,329; 4,540,783; 4,550,109; 4,587,340; 4,595,752; 4,649,197; 4,665,182; 4,701,455; 4,713,454; 4,937,342; 5,037,981; 5,198,547; 5,350,851; 5,401,844; 5,698,408 and Canadian application CA 2420374 (each incorporated herein by reference) each describe methods of making dihydrobiopterins, BH4 and derivative thereof that may be used as compositions for the present invention. Any such methods may be used to produce BH4 compositions for use in the therapeutic methods of the present invention.

U.S. Pat. Nos. 4,752,573; 4,758,571; 4,774,244; 4,920,122; 5,753,656; 5,922,713; 5,874,433; 5,945,452; 6,274,581; 6,410,535; 6,441,038; 6,544,994; and U.S. Patent Publications US 20020187958; US 20020106645; US 2002/0076782; US 20030032616(each incorporated herein by reference) each describe methods of administering BH4 compositions for various treatments. Each of those patents is incorporated herein by reference as providing a general teaching of methods of administering BH4 compositions known to those of skill in the art, that may be adapted for the treatment of vascular diseases described herein.

In particular, U.S. Pat. No. 4,540,783 describes BH4 derivatives that are 1′,2′-diacyl-(6R,S)-5,6,7,8-tetrahydro-L-biopterins, and inorganic or organic salts thereof, that are useful according to the therapeutic methods of the invention. Preferably pharmaceutically acceptable salts are used for therapeutic methods of the invention. The compounds described in U.S. Pat. No. 4,540,783 have the general formula (I):

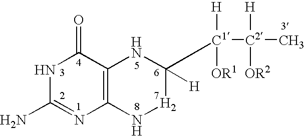

wherein R¹ and R² are the same or different and each is an acyl group.

The acyl group has preferably 1 to 10 carbon atoms, in particular 3 to 10 carbon atoms. Preferable acyl group is represented by the general formula R⁵CO— wherein R⁵ is hydrogen or a hydrocarbon residue having 1 or more carbon atoms, in particular 2 to 9 carbon atoms. Preferable examples of the hydrocarbon residue represented by R⁵ are, for instance, a linear or branched alkyl group having 1 or more carbon atoms, preferably 2 to 9 carbon atoms, which is either saturated or unsaturated; a substituted or unsubstituted phenyl group represented by the general formula

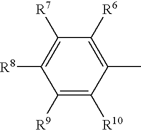

wherein R⁶, R⁷, R⁸, R⁹ and R¹⁰ are hydrogen or a linear or branched alkyl group wherein the combined number of carbon atoms is R⁶, R⁷, R⁸, R⁹, R¹⁰ is preferably not more than 3; a substituted or unsubstituted benzyl group represented by the general formula

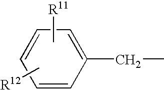

wherein R¹¹ and R¹² are hydrogen, methyl or ethyl wherein the combined number of carbon atoms R¹¹ and R¹² is preferably not more than 2; and a substituted or unsubstituted arylalkyl group represented by the general formula

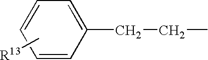

wherein R¹³ is hydrogen or methyl group. Among the above acyl groups, formyl, acetyl, propionyl, butyryl, isobutyryl, valeryl, isovaleryl and benzoyl are most preferable. It is preferable that R¹ and R² are the same.

The compound of the general formula (I) has two diastereomers, i.e. 1′,2′-diacyl-(6R)-5,6,7,8-tetrahydro-L-biopterin and 1′,2′-diacyl-(6S)-5,6,7,8-tetrahydro-L-biopterin which are diastereomeric at the 6 position. The compound of the present invention includes the two diastereomers and a mixture thereof.

The compound (I) of the present invention can be in a form of an inorganic salt such as a hydrochloride, a sulfate or a phosphate, an organic salt such as an acetate, an oxalate, or a complex salt.

U.S. Pat. No. 4,550,109 describes BH4 derivatives that are lipoidal biopterins and tetrahydrobiopterins. These lipoidal BH4 derivatives may be administered as pharmaceutically acceptable salts according to the therapeutic methods of the invention. The compounds described in U.S. Pat. No. 4,550,109 are represented by the following structure:

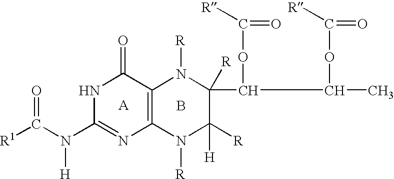

wherein
—R is absent when there are two double bonds in ring B;
—R is hydrogen when the two double bonds in ring B are absent; and
—R′ and R″ are independently saturated or unsaturated, aliphatic hydrocarbon groups which are balanced in molecular weight such that they confer to the lipoidal compound a lipoidal property.

Generally R′ and R″ are selected from hydrocarbons having from 1 to 31 carbon units, with the limitation that the sum of carbon units of R′+2R″ is greater than 10 but less than 33.

In these derivatives, the 2-N-acyl group is desirably from 9 to 32 and preferably 9 to 20 carbon units so as to confer lipoidal characteristics upon the final product. The 2-N-acyl group is exemplified by decanolyl-, palmitoyl-, stearoyl- and linoleyl. The 2-N-acyl group may be saturated as is stearoyl- or unsaturated as is linoleyl. In addition, non-toxic aromatic 2-N-acyl groups like phenylacetyl can also confer the desirable lipoidal characteristics to the final product. The 1′,2′-di-O-acyl groups, are desirably lower molecular weight alkyls and alkenyls having from 2 to 8 and preferably 2 to 4 carbon units, with acetyl being exemplary.

U.S. Pat. No. 4,550,109 also describes biopterin compounds of the formula:

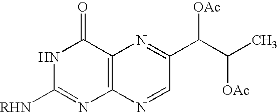

where R is a naturally occurring fatty acid, which can be saturated or unsaturated, and Ac COCH₃. These biopterin compounds can be hydrogenated to form the corresponding tetrahydrobiopterins, which are useful according to the therapeutic methods of the invention. Exemplary chain lengths of the group R fatty acid range from C₁₀ to C₁₈ units. Exemplary compounds include 2N-Acetyl-1′,2′-di-O-Acetyl-L-Biopterin, 2-N-Decanoyl-1′,2′-di-O-acetyl-L-biopterin, 2-N-Palmitoyl-1′,2′-di-O-acetyl-L-biopterin, 2-N-Stearoyl-1′,2′-di-O-acetyl-L-biopterin, 2-N-Linoleyl-1′,2′-di-O-acetyl-L-biopterin, and 2-N-Phenylacetyl-1′,2′-di-O-acetyl-L-biopterin, and the corresponding tetrahydrobiopterins.

In addition to the above general methods of making BH4, the present invention particularly contemplates making and using a BH4 composition which is a stabilized BH4 composition, preferably a 99.5% or 99.9% pure 6R BH4. If BH4 itself is being administered, any of the salts or polymorph or crystalline forms described in U.S. patent application Ser. No. 11/143,887 (and counterpart Int'l Application No. PCT/US04/38296 filed Nov. 17, 2004, published as WO 2005/049000) and in U.S. patent application Ser. No. 10/990,316 (and counterpart Int'l Application No. PCT/IB04/04447 filed Nov. 17, 2004, published as WO 2005/065018) may be administered in purified form. The various crystalline forms may conveniently be formed into a tablets, powder or other solid for oral administration. The crystalline forms may also prove useful as an additive to conventional foodstuffs. The BH4 may be administered as a stable solid formulation as described in Int'l Application No. PCT/US05/41252 filed Nov. 16, 2005, published as WO 2006/055511, incorporated herein by reference in its entirety. The forms and routes of administration of BH4 are discussed in further detail below.

In preferred embodiments, it is contemplated that the methods of the present invention will provide to a patient in need thereof, a daily dose of between about 10 mg/kg to about 20 mg/kg of BH4. Of course, one skilled in the art may adjust this dose up or down depending on the efficacy being achieved by the administration. The daily dose may be administered in a single dose or alternatively may be administered in multiple doses at conveniently spaced intervals. In exemplary embodiments, the daily dose may be 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 22 mg/kg, 24 mg/kg, 26 mg/kg, 28 mg/kg, 30 mg/kg, 32 mg/kg, 34 mg/kg, 36 mg/kg, 38 mg/kg, 40 mg/kg, 42 mg/kg, 44 mg/kg, 46 mg/kg, 48 mg/kg, 50 mg/kg, or more mg/kg.

In the low dose therapeutic methods of the invention, low doses, e.g., doses of 0.1 to 5 mg/kg per day are contemplated, including doses of 0.1 to 2 mg/kg, or 0.1 to 3 mg/kg, or 1 mg/kg to 5 mg/kg. Doses of less than 5 mg/kg per day are preferred. According to the invention, such doses are expected to provide improvements with relevant study endpoints, and BH4 derivatives are expected to have improved biological properties relative to natural BH4 at such doses. In particular, the invention contemplates that any of the 1′,2′-diacyl-(6R,S)-5,6,7,8-tetrahydro-L-biopterins or lipoidal tetrahydrobiopterins described herein exhibit improved biological properties at low doses.

The invention specifically contemplates the use of BH4, or a precursor or derivative thereof, for treating any of the disease states mentioned in the present application or any of the vascular disease states mentioned in U.S. application Ser. No. 11/143,887 filed Jun. 1, 2005 (and counterpart Int'l Application No. PCT/US04/38296 filed Nov. 17, 2004, published as WO 2005/049000), incorporated herein by reference in its entirety, at a dose in the range of 0.1 to 5 mg/kg body weight/day, via any route of administration including but not limited to oral administration, in a once daily dose or multiple (e.g. 2, 3 or 4) divided doses per day, for a duration of at least 1, 2, 3, or 4 weeks or longer, or 1, 2, 3, 4, 5, 6 months or longer. Exemplary doses include less than 5 mg/kg/day, 4.5 mg/kg/day or less, 4 mg/kg/day or less, 3.5 mg/kg/day or less, 3 mg/kg/day or less, 2.5 mg/kg/day or less, 2 mg/kg/day or less, 1.5 mg/kg/day or less, 1 mg/kg/day or less, or 0.5 mg/kg/day or less. Equivalent doses per body surface area are also contemplated.

For the person of average weight/body surface area (e.g. 70 kg), the invention also contemplates a total daily dose of less than 400 mg. Exemplary such total daily doses include 360 mg/day, 350 mg/day, 300 mg/day, 280 mg/day, 210 mg/day, 180 mg/day, 175 mg/day, 150 mg/day, or 140 mg/day. For example, 350 mg/day or 175 mg/day is easily administrable with an oral dosage formulation of 175 mg, once or twice a day. Other exemplary total daily doses include 320 mg/day or less, 160 mg/day or less, or 80 mg/day or less. Such doses are easily administrable with an oral dosage formulation of 80 or 160 mg. Other exemplary total daily doses include 45, 90, 135, 180, 225, 270, 315 or 360 mg/day or less, easily administrable with an oral dosage formulation of 45 or 90 mg. Yet other exemplary total daily doses include 60, 120, 180, 240, 300, or 360 mg/day, easily administrable with an oral dosage formulation of 60 or 120 mg. Other exemplary total daily doses include 70, 140, 210, 280, or 350 mg/day, easily administrable with an oral dosage formulation of 70 or 140 mg. Exemplary total daily doses also include 55, 110, 165, 220, 275 or 330 mg/day, easily administrable with an oral dosage formulation of 55 mg. Other exemplary total daily doses include 65, 130, 195, 260, or 325 mg/day, or 75, 150, 225, 300 or 375 mg/day, e.g. in dosage formulations of 65 mg or 75 mg.

### 3. Pharmaceutical Compositions

Pharmaceutical compositions for administration according to the present invention can comprise a first composition comprising BH4 in a pharmaceutically acceptable form optionally combined with a pharmaceutically acceptable carrier. These compositions can be administered by any means that achieve their intended purposes. Amounts and regimens for the administration of a composition according to the present invention can be determined readily by those with ordinary skill in the art for treating vascular disease. As discussed above, those of skill in the art could initially employ amounts and regimens of BH4 currently being proposed in a medical context, e.g., those compositions that are being proposed for modulating NOS activity. Any of the protocols, formulations, routes of administration and the like described that have been used for administering BH4 for loading tests can readily be modified for use in the present invention.

Compositions within the scope of this invention include all compositions comprising BH4, analogs and derivative thereof according to the present invention in an amount effective to achieve its intended purpose. Similarly, as certain therapeutic methods of the present invention contemplate a combination therapy in which BH4-based compositions are administered in addition to agents and interventions commonly used to treat vascular disease, the pharmaceutical compositions of the invention also contemplate all compositions comprising at least BH4-based therapeutic agent, analog or homologue thereof in an amount effective to achieve the amelioration of one or more of the symptoms of vascular disease when administered in combination with the conventional agents and interventions used to treat vascular disease. Of course, the most obvious symptom that may be alleviated is that the combined therapy produces improvement in clinically relevant endpoints, however, other symptoms and the like also may be monitored. Such indicia are monitored using techniques known to those of skill in the art.

Crystal Polymorphs of (6R) L-Tetrahydrobiopterin Dihydrochloride Salt

It has been found that BH4, and in particular, the dihydrochloride salt of BH4, exhibits crystal polymorphism. The structure of BH4 is shown below:

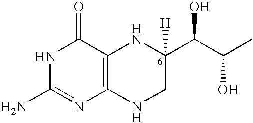

The (6R) form of BH4 is the known biologically active form, however, BH4 is also known to be unstable at ambient temperatures. It has been found that one crystal polymorph of BH4 is more stable, and is stable to decomposition under ambient conditions.

BH4 is difficult to handle and it is therefore produced and offered as its dihydrochloride salt (Schircks Laboratories, Jona, Switzerland) in ampoules sealed under nitrogen to prevent degradation of the substance due to its hygroscopic nature and sensitivity to oxidation. U.S. Pat. No. 4,649,197 discloses that separation of (6R)- and 6(S)-L-erythro-tetrahydrobiopterin dihydrochloride into its diastereomers is difficult due to the poor crystallinity of 6(R,S)-L-erythro-tetrahydrobiopterin dihydrochloride. The European patent number 0 079 574 describes the preparation of tetrahydrobiopterin, wherein a solid tetrahydrobiopterin dihydrochloride is obtained as an intermediate. S. Matsuura et al. describes in Chemistry Letters 1984, pages 735-738 and Heterocycles, Vol. 23, No. 12, 1985 pages 3115-3120 6(R)-tetrahydrobiopterin dihydrochloride as a crystalline solid in form of colorless needles, which are characterized by X-ray analysis disclosed in J. Biochem. 98, 1341-1348 (1985). An optical rotation of 6.81° was found the crystalline product, which is quite similar to the optical rotation of 6.51° reported for a crystalline solid in form of white crystals in example 6 of EP-A2-0 191 335.

Results obtained during development of (6R)-L-erythro-tetrahydrobiopterin dihydrochloride indicated that the compound may exist in different crystalline forms, including polymorphic forms and solvates. The continued interest in this area requires an efficient and reliable method for the preparation of the individual crystal forms of (6R)-L-erythro-tetrahydrobiopterin dihydrochloride and controlled crystallization conditions to provide crystal forms, that are preferably stable and easy to handle and to process in the manufacture and preparation of formulations, and that provide a high storage stability in substance form or as formulated product, or which provide less stable forms suitable as intermediates for controlled crystallization for the manufacture of stable forms.

### Polymorph Form B

The crystal polymorph that has been found to be the most stable is referred to herein as “form B,” or alternatively as “polymorph B.” Results obtained during investigation and development of (6R)-L-erythro-tetrahydrobiopterin dihydrochloride development revealed that there are several known crystalline solids have been prepared, but none have recognized the polymorphism and its effect on the stability of the BH4 crystals.

Polymorph B is a slightly hygroscopic anhydrate with the highest thermodynamic stability above about 20° C. Furthermore, form B can be easily processed and handled due to its thermal stability, possibility for preparation by targeted conditions, its suitable morphology and particle size. Melting point is near 260° C. (ΔHf>140 J/g), but no clear melting point can be detected due to decomposition prior and during melting. These outstanding properties render polymorph form B especially feasible for pharmaceutical application, which are prepared at elevated temperatures. Polymorph B can be obtained as a fine powder with a particle size that may range from 0.2 μm to 500 μm.

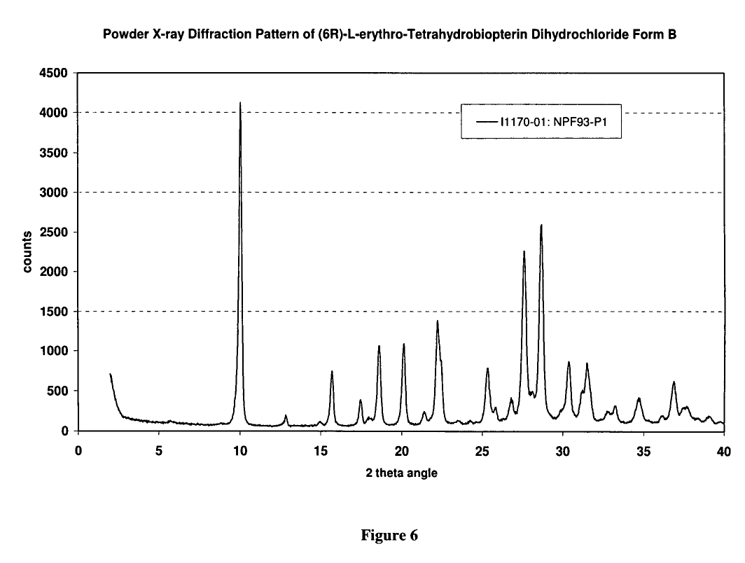

Form B exhibits an X-ray powder diffraction pattern, expressed in d-values (Å) at: 8.7 (vs), 6.9 (w), 5.90 (vw), 5.63 (m), 5.07 (m), 4.76 (m), 4.40 (m), 4.15 (w), 4.00 (s), 3.95 (m), 3.52 (m), 3.44 (w), 3.32 (m), 3.23 (s), 3.17 (w), 3.11 (vs), 3.06 (w), 2.99 (w), 2.96 (w), 2.94 (m), 2.87 (w), 2.84 (s), 2.82 (m), 2.69 (w), 2.59 (w), 2.44 (w). FIG. 6 is a graph of the characteristic X-ray diffraction pattern exhibited by form B of (6R)-L-erythro-tetrahydrobiopterin dihydrochloride.

As used herein, the following the abbreviations in brackets mean: (vs)=very strong intensity; (s)=strong intensity; (m)=medium intensity; (w)=weak intensity; and (vw)=very weak intensity. A characteristic X-ray powder diffraction pattern is exhibited in FIG. 6.

It has been found that other polymorphs of BH4 have a satisfactory chemical and physical stability for a safe handling during manufacture and formulation as well as providing a high storage stability in its pure form or in formulations. In addition, it has been found that form B, and other polymorphs of BH4 can be prepared in very large quantities (e.g., 100 kilo scale) and stored over an extended period of time.

All crystal forms (polymorphs, hydrates and solvates), inclusive crystal form B, can be used for the preparation of the most stable polymorph B. Polymorph B may be obtained by phase equilibration of suspensions of amorphous or other forms than polymorph form B, such as polymorph A, in suitable polar and non aqueous solvents. Thus, the pharmaceutical preparations described herein refer to a preparation of polymorph form B of (6R)-L-erythro-tetrahydrobiopterin dihydrochloride.

Other forms of BH4 can be converted for form B by dispersing the other form of BH4 in a solvent at room temperature, stirring the suspension at ambient temperatures for a time sufficient to produce polymorph form B, thereafter isolating crystalline form B and removing the solvent from the isolated form B. Ambient temperatures, as used herein, mean temperatures in a range from 0° C. to 60° C., preferably 15° C. to 40° C. The applied temperature may be changed during treatment and stirring by decreasing the temperature stepwise or continuously. Suitable solvents for the conversion of other forms to form B include but are not limited to, methanol, ethanol, isopropanol, other C3- and C4-alcohols, acetic acid, acetonitrile, tetrahydrofurane, methyl-t-butyl ether, 1,4-dioxane, ethyl acetate, isopropyl acetate, other C3-C6-acetates, methyl ethyl ketone and other methyl-C3-C5 alkyl-ketones. The time to complete phase equilibration may be up to 30 hours and preferably up to 20 hours or less than 20 hours.

Polymorph B may also be obtained by crystallization from solvent mixtures containing up to about 5% water, especially from mixtures of ethanol, acetic acid and water. It has been found that polymorph form B of (6R)-L-erythro-tetrahydrobiopterin dihydrochloride can be prepared by dissolution, optionally at elevated temperatures, preferably of a solid lower energy form than form B or of form B of (6R)-L-erythro-tetrahydrobiopterin dihydrochloride in a solvent mixture comprising ethanol, acetic acid and water, addition of seeds to the solution, cooling the obtained suspension and isolation of the formed crystals. Dissolution may be carried out at room temperature or up to 70° C., preferably up to 50° C. There may be used the final solvent mixture for dissolution or the starting material may be first dissolved in water and the other solvents may than be added both or one after the other solvent. The composition of the solvent mixture may comprise a volume ratio of water:acetic acid:tetrahydrofuran of 1:3:2 to 1:9:4 and preferably 1:5:4. The solution is preferably stirred. Cooling may mean temperatures down to −40° C. to 0° C., preferably down to 10° C. to 30° C. Suitable seeds are polymorph form B from another batch or crystals having a similar or identical morphology. After isolation, the crystalline form B can be washed with a non-solvent such as acetone or tetrahydrofurane and dried in usual manner.

Polymorph B may also be obtained by crystallization from aqueous solutions through the addition of non-solvents such as methanol, ethanol and acetic acid. The crystallization and isolation procedure can be advantageously carried out at room temperature without cooling the solution. This process is therefore very suitable to be carried out at an industrial scale.

In one embodiment of the compositions and methods described herein, a composition including polymorph form B of (6R)-L-erythro-tetrahydrobiopterin dihydrochloride is prepared by dissolution of a solid form other than form B or of form B of (6R)-L-erythro-tetrahydrobiopterin dihydrochloride in water at ambient temperatures, adding a non-solvent in an amount sufficient to form a suspension, optionally stirring the suspension for a certain time, and thereafter isolation of the formed crystals. The composition is further modified into a pharmaceutical composition as described below.

The concentration of (6R)-L-erythro-tetrahydrobiopterin dihydrochloride in the aqueous solution may be from 10 to 80 percent by weight, more preferably from 20 to 60 percent by weight, by reference to the solution. Preferred non-solvents (i.e., solvents useful in preparing suspensions of BH4) are methanol, ethanol and acetic acid. The non-solvent may be added to the aqueous solution. More preferably, the aqueous solution is added to the non-solvent. The stirring time after formation of the suspension may be up to 30 hours and preferably up to 20 hours or less than 20 hours. Isolation by filtration and drying is carried out in known manner as described above.

Polymorph form B is a very stable crystalline form that can be easily filtered off, dried and ground to particle sizes desired for pharmaceutical formulations. These outstanding properties render polymorph form B especially feasible for pharmaceutical application.

### Polymorph Form A

It has been found that another crystal polymorph of (6R)-L-erythro-tetrahydrobiopterin dihydrochloride is a stable preferred form of BH4 for use in a pharmaceutical preparation described herein, which shall be referred to herein as “form A,” or “polymorph A.” Polymorph A is slightly hygroscopic and adsorbs water to a content of about 3 percent by weight, which is continuously released between 50° C. and 200° C., when heated at a rate of 10° C./minute. The polymorph A is a hygroscopic anhydrate, which is a meta-stable form with respect to form B; however, it is stable over several months at ambient conditions if kept in a tightly sealed container. Form A is especially suitable as intermediate and starting material to produce stable polymorph forms. Polymorph form A can be prepared as a solid powder with desired medium particle size range which is typically ranging from 1 μm to about 500 μm.

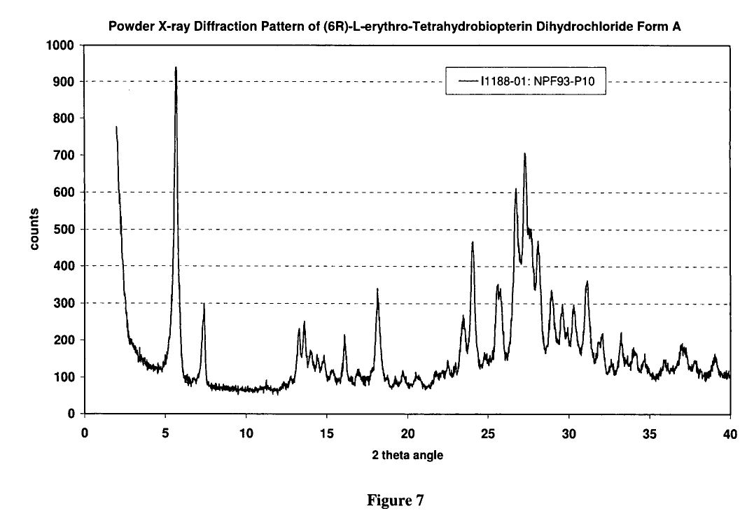

Polymorph A which exhibits a characteristic X-ray powder diffraction pattern with characteristic peaks expressed in d-values (A) of: 15.5 (vs.), 12.0 (m), 6.7 (m), 6.5 (m), 6.3 (w), 6.1 (w), 5.96 (w), 5.49 (m), 4.89 (m), 3.79 (m), 3.70 (s), 3.48 (m), 3.45 (m), 3.33 (s), 3.26 (s), 3.22 (m), 3.18 (m), 3.08 (m), 3.02 (w), 2.95 (w), 2.87 (m), 2.79 (w), 2.70 (w). FIG. 7 is a graph of the characteristic X-ray diffraction pattern exhibited by form A of (6R)-L-erythro-tetrahydrobiopterin dihydrochloride.

Polymorph A exhibits a characteristic Raman spectra bands, expressed in wave numbers (cm-1) at: 2934 (w), 2880 (w), 1692 (s), 1683 (m), 1577 (w), 1462 (m), 1360 (w), 1237 (w), 1108 (w), 1005 (vw), 881 (vw), 813 (vw), 717 (m), 687 (m), 673 (m), 659 (m), 550 (w), 530 (w), 492 (m), 371 (m), 258 (w), 207 (w), 101 (s), 87 (s) cm-1.

Polymorph form A may be obtained by freeze-drying or water removal of solutions of (6R)-L-erythro-tetrahydrobiopterin dihydrochloride in water. Polymorph form A of (6R)-L-erythro-tetrahydrobiopterin dihydrochloride can be prepared by dissolving (6R)-L-erythro-tetrahydrobiopterin dihydrochloride at ambient temperatures in water, (1) cooling the solution to low temperatures for solidifying the solution, and removing water under reduced pressure, or (2) removing water from said aqueous solution.

The crystalline form A can be isolated by filtration and then dried to evaporate absorbed water from the product. Drying conditions and methods are known and drying of the isolated product or water removal pursuant to variant (2) described herein may be carried out in applying elevated temperatures, for example up to 80° C., preferably in the range from 30° C. to 80° C., under vacuum or elevated temperatures and vacuum. Prior to isolation of a precipitate obtained in variant (2), the suspension may be stirred for a certain time for phase equilibration. The concentration of (6R)-L-erythro-tetrahydrobiopterin dihydrochloride in the aqueous solution may be from 5 to 40 percent by weight, by reference to the solution.

A fast cooling is preferred to obtain solid solutions as starting material. A reduced pressure is applied until the solvent is completely removed. Freeze drying is a technology well known in the art. The time to complete solvent removal is dependent on the applied vacuum, which may be from 0.01 to 1 mbar, the solvent used and the freezing temperature.

Polymorph form A is stable at room temperature or below room temperature under substantially water free conditions, which is demonstrated with phase equilibration tests of suspensions in tetrahydrofuran or tertiary-butyl methyl ether stirred for five days and 18 hours respectively under nitrogen at room temperature. Filtration and air-drying at room temperature yields unchanged polymorph form A.

### Polymorph Form F

It has been found that another crystal polymorph of (6R)-L-erythro-tetrahydrobiopterin dihydrochloride is a stable preferred form of BH4 for use in a pharmaceutical preparation described herein, which shall be referred to herein as “form F,” or “polymorph F.” Polymorph F is slightly hygroscopic and adsorbs water to a content of about 3 percent by weight, which is continuously released between 50° C. and 200° C., when heated at a rate of 10° C./minute. The polymorph F is a meta-stable form and a hygroscopic anhydrate, which is more stable than form A at ambient lower temperatures and less stable than form B at higher temperatures and form F is especially suitable as intermediate and starting material to produce stable polymorph forms. Polymorph form F can be prepared as a solid powder with desired medium particle size range which is typically ranging from 1 μm to about 500 μm.

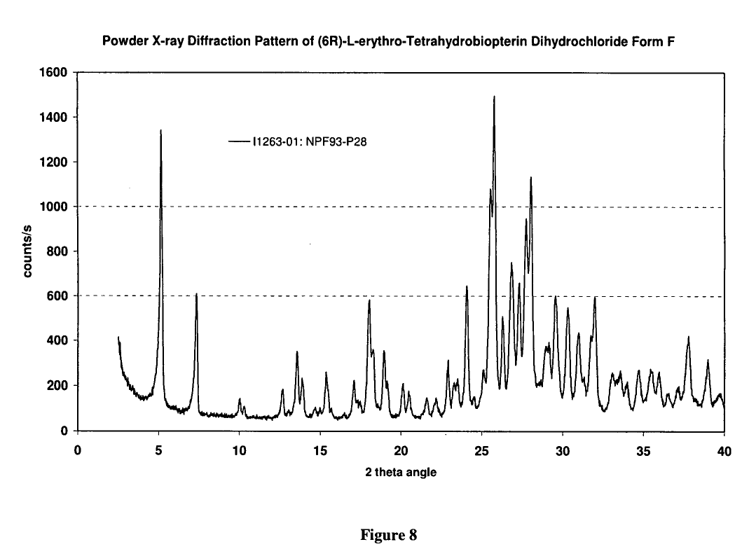

Polymorph F exhibits a characteristic X-ray powder diffraction pattern with characteristic peaks expressed in d-values (Å) at: 17.1 (vs.), 12.1 (w), 8.6 (w), 7.0 (w), 6.5 (w), 6.4 (w), 5.92 (w), 5.72 (w), 5.11 (w), 4.92 (m), 4.86 (w), 4.68 (m), 4.41 (w), 4.12 (w), 3.88 (w), 3.83 (w), 3.70 (m), 3.64 (w), 3.55 (m), 3.49 (s), 3.46 (vs), 3.39 (s), 3.33 (m), 3.31 (m), 3.27 (m), 3.21 (m), 3.19 (m), 3.09 (m), 3.02 (m), and 2.96 (m). FIG. 8 is a graph of the characteristic X-ray diffraction pattern exhibited by form F of (6R)-L-erythro-tetrahydrobiopterin dihydrochloride.

Polymorph F may be obtained by phase equilibration of suspensions of polymorph form A in suitable polar and non-aqueous solvents, which scarcely dissolve said lower energy forms, especially alcohols such as methanol, ethanol, propanol and isopropanol. Polymorph form F of (6R)-L-erythro-tetrahydrobiopterin dihydrochloride can also be prepared by dispersing particles of solid form A of (6R)-L-erythro-tetrahydrobiopterin dihydrochloride in a non-aqueous solvent that scarcely dissolves said (6R)-L-erythro-tetrahydrobiopterin dihydrochloride below room temperature, stirring the suspension at said temperatures for a time sufficient to produce polymorph form F, thereafter isolating crystalline form F and removing the solvent from the isolated form F. Removing of solvent and drying may be carried out under air, dry air or a dry protection gas such as nitrogen or noble gases and at or below room temperature, for example down to 0° C. The temperature during phase equilibration is preferably from 5 to 15° C. and most preferably about 10° C.

### Polymorph Form J

It has been found that another crystal polymorph of (6R)-L-erythro-tetrahydrobiopterin dihydrochloride is a stable preferred form of BH4 for use in a pharmaceutical preparation described herein, which shall be referred to herein as “form J,” or “polymorph J.” The polymorph J is slightly hygroscopic and adsorbs water when handled at air humidity. The polymorph J is a meta-stable form and a hygroscopic anhydrate, and it can be transformed back into form E described below, from which it is obtained upon exposure to high relative humidity conditions such as above 75% relative humidity. Form J is especially suitable as intermediate and starting material to produce stable polymorph forms. Polymorph form J can be prepared as a solid powder with desired medium particle size range which is typically ranging from 1 μm to about 500 μm.

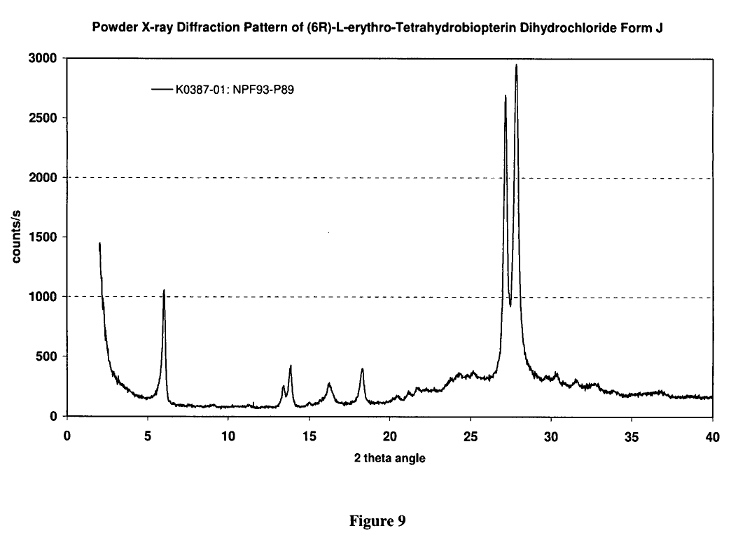

Form J exhibits a characteristic X-ray powder diffraction pattern with characteristic peaks expressed in d-values (Å) at: 14.6 (m), 6.6 (w), 6.4 (w), 5.47 (w), 4.84 (w), 3.29 (vs), and 3.21 (vs). FIG. 9 is a graph of the characteristic X-ray diffraction pattern exhibited by form J of (6R)-L-erythro-tetrahydrobiopterin dihydrochloride.

Polymorph J may be obtained by dehydration of form E at moderate temperatures under vacuum. In particular, polymorph form J of (6R)-L-erythro-tetrahydrobiopterin dihydrochloride can be prepared by taking form E and removing the water from form E by treating form E in a vacuum drier to obtain form J at moderate temperatures, which may mean a temperature in the range of 25 to 70° C., and most preferably 30 to 50° C.

### Polymorph Form K

It has been found that another crystal polymorph of (6R)-L-erythro-tetrahydrobiopterin dihydrochloride is a stable preferred form of BH4 for use in a pharmaceutical preparation described herein, which shall be referred to herein as “form K,” or “polymorph K.” Polymorph K is slightly hygroscopic and adsorbs water to a content of about 2.0 percent by weight, which is continuously released between 50° C. and 100° C., when heated at a rate of 10° C./minute. The polymorph K is a meta-stable form and a hygroscopic anhydrate, which is less stable than form B at higher temperatures and form K is especially suitable as intermediate and starting material to produce stable polymorph forms, in particular form B. Polymorph form K can be prepared as a solid powder with desired medium particle size range which is typically ranging from 1 μm to about 500 μm.

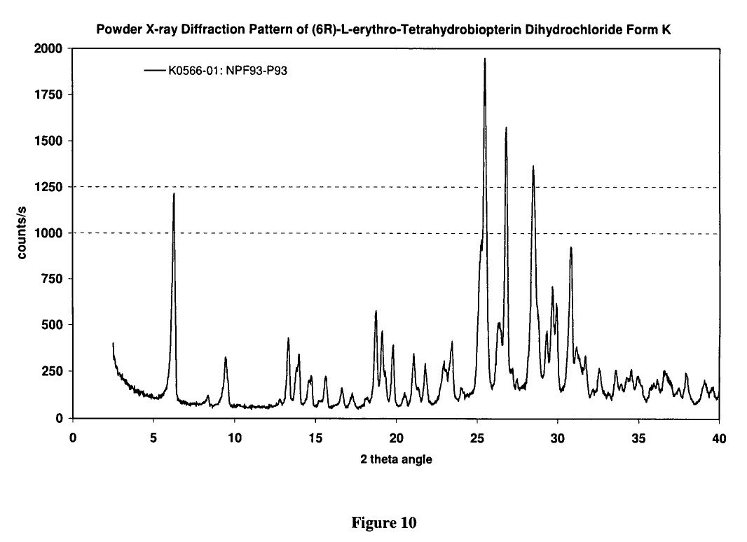

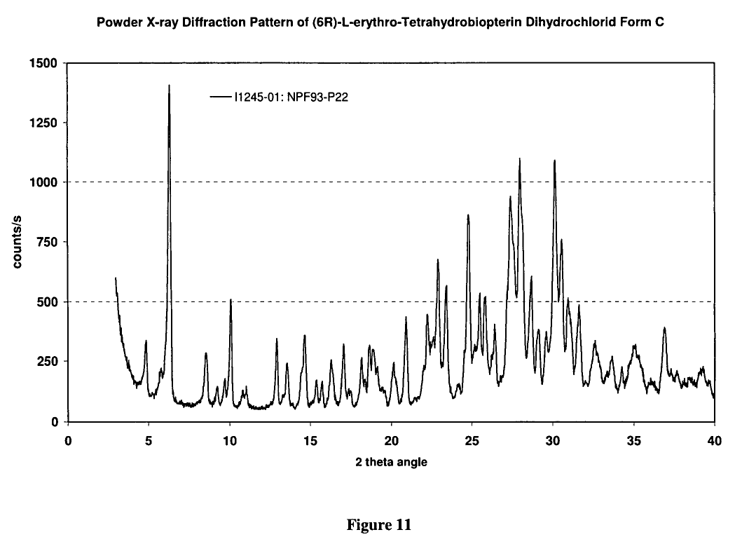

Form K exhibits a characteristic X-ray powder diffraction pattern with characteristic peaks expressed in d-values (Å) at: 14.0 (s), 9.4 (w), 6.6 (w), 6.4 (w), 6.3 (w), 6.1 (w), 6.0 (w), 5.66 (w), 5.33 (w), 5.13 (vw), 4.73 (m), 4.64 (m), 4.48 (w), 4.32 (vw), 4.22 (w), 4.08 (w), 3.88 (w), 3.79 (w), 3.54 (m), 3.49 (vs), 3.39 (m), 3.33 (vs), 3.13 (s), 3.10 (m), 3.05 (m), 3.01 (m), 2.99 (m), and 2.90 (m). FIG. 10 is a graph of the characteristic X-ray diffraction pattern exhibited by form K of (6R)-L-erythro-tetrahydrobiopterin dihydrochloride.

Polymorph K may be obtained by crystallization from mixtures of polar solvents containing small amounts of water and in the presence of small amounts of ascorbic acid. Solvents for the solvent mixture may be selected from acetic acid and an alcohol such as methanol, ethanol, n- or isopropanol. In particular, polymorph form K of (6R)-L-erythro-tetrahydrobiopterin dihydrochloride can be prepared by dissolving (6R)-L-erythro-tetrahydrobiopterin dihydrochloride in a mixture of acetic acid and an alcohol or tetrahydrofuran containing small amounts of water and a small amount of ascorbic acid at elevated temperatures, lowering temperature below room temperature to crystallize said dihydrochloride, isolating the precipitate and drying the isolated precipitate at elevated temperature optionally under vacuum. Suitable alcohols are for example methanol, ethanol, propanol and isopropanol, whereby ethanol is preferred. The ratio of acetic acid to alcohol or tetrahydrofuran may be from 2:1 to 1:2 and preferably about 1:1. Dissolution of (6R)-L-erythro-tetrahydrobiopterin dihydrochloride can be carried out in presence of a higher water content and more of the anti-solvent mixture can be added to obtain complete precipitation. The amount of water in the final composition may be from 0.5 to 5 percent by weight and the amount of ascorbic acid may be from 0.01 to 0.5 percent by weight, both by reference to the solvent mixture. The temperature for dissolution may be in the range from 30 to 100 and preferably 35 to 70° C. and the drying temperature may be in the range from 30 to 50° C. The precipitate may be washed with an alcohol such as ethanol after isolation, e.g., filtration. The polymorph K can easily be converted in the most stable form B by phase equilibration in e.g., isopropanol and optionally seeding with form B crystals at above room temperature such as temperatures from 30 to 40° C.

Hydrate Forms of (6R) L-Tetrahydrobiopterin Dihydrochloride Salt

As further described below, it has been found that (6R)-L-erythro-tetrahydrobiopterin dihydrochloride exists as a number of crystalline hydrate, which shall be described and defined herein as forms C, D, E, H, and O. These hydrate forms are useful as a stable form of BH4 for the pharmaceutical preparations described herein and in the preparation of compositions including stable crystal polymorphs of BH4.

### Hydrate Form C

It has been found that a hydrate crystal form of (6R)-L-erythro-tetrahydrobiopterin dihydrochloride is a stable preferred form of BH4 for use in a pharmaceutical preparation described herein, which shall be referred to herein as “form C,” or “hydrate C.” The hydrate form C is slightly hygroscopic and has a water content of approximately 5.5 percent by weight, which indicates that form C is a monohydrate. The hydrate C has a melting point near 94° C. (ΔH_{f} is about 31 J/g) and hydrate form C is especially suitable as intermediate and starting material to produce stable polymorphic forms. Polymorph form C can be prepared as a solid powder with desired medium particle size range which is typically ranging from 1 μm to about 500 μm.

Form C exhibits a characteristic X-ray powder diffraction pattern with characteristic peaks expressed in d-values (Å) at: 18.2 (m), 15.4 (w), 13.9 (vs), 10.4 (w), 9.6 (w), 9.1 (w), 8.8 (m), 8.2 (w), 8.0 (w), 6.8 (m), 6.5 (w), 6.05 (m), 5.77 (w), 5.64 (w), 5.44 (w), 5.19 (w), 4.89 (w), 4.76 (w), 4.70 (w), 4.41 (w), 4.25 (m), 4.00 (m), 3.88 (m), 3.80 (m), 3.59 (s), 3.50 (m), 3.44 (m), 3.37 (m), 3.26 (s), 3.19 (vs), 3.17 (s), 3.11 (m), 3.06 (m), 3.02 (m), 2.97 (vs), 2.93 (m), 2.89 (m), 2.83 (m), and 2.43 (m). FIG. 11 is a graph of the characteristic X-ray diffraction pattern exhibited by hydrate form C of (6R)-L-erythro-tetrahydrobiopterin dihydrochloride.

Hydrate form C may be obtained by phase equilibration at ambient temperatures of a polymorph form such as polymorph B suspension in a non-solvent, which contains water in an amount of preferably about 5 percent by weight, by reference to the solvent. Hydrate form C of (6R)-L-erythro-tetrahydrobiopterin dihydrochloride cab be prepared by suspending (6R)-L-erythro-tetrahydrobiopterin dihydrochloride in a non-solvent such as, heptane, C1-C4-alcohols such as methanol, ethanol, 1- or 2-propanol, acetates, such as ethyl acetate, acetonitrile, acetic acid or ethers such as terahydrofuran, dioxane, tertiary-butyl methyl ether, or binary or ternary mixtures of such non-solvents, to which sufficient water is added to form a monohydrate, and stirring the suspension at or below ambient temperatures (e.g., 0 to 30° C.) for a time sufficient to form a monohydrate. Sufficient water may mean from 1 to 10 and preferably from 3 to 8 percent by weight of water, by reference to the amount of solvent. The solids may be filtered off and dried in air at about room temperature. The solid can absorb some water and therefore possess a higher water content than the theoretical value of 5.5 percent by weight. Hydrate form C is unstable with respect to forms D and B, and easily converted to polymorph form B at temperatures of about 40° C. in air and lower relative humidity. Form C can be transformed into the more stable hydrate D by suspension equilibration at room temperature.

### Hydrate Form D

It has been found that another hydrate crystal form of (6R)-L-erythro-tetrahydrobiopterin dihydrochloride is a stable preferred form of BH4 for use in a pharmaceutical preparation described herein, which shall be referred to herein as “form D,” or “hydrate D.” The hydrate form D is slightly hygroscopic and may have a water content of approximately 5.0 to 7.0 percent by weight, which suggests that form D is a monohydrate. The hydrate D has a melting point near 153° C. (ΔH_{f} is about 111 J/g) and is of much higher stability than form C and is even stable when exposed to air humidity at ambient temperature. Hydrate form D can therefore either be used to prepare formulations or as intermediate and starting material to produce stable polymorph forms. Polymorph form D can be prepared as a solid powder with desired medium particle size range which is typically ranging from 1 μm to about 500 μm.

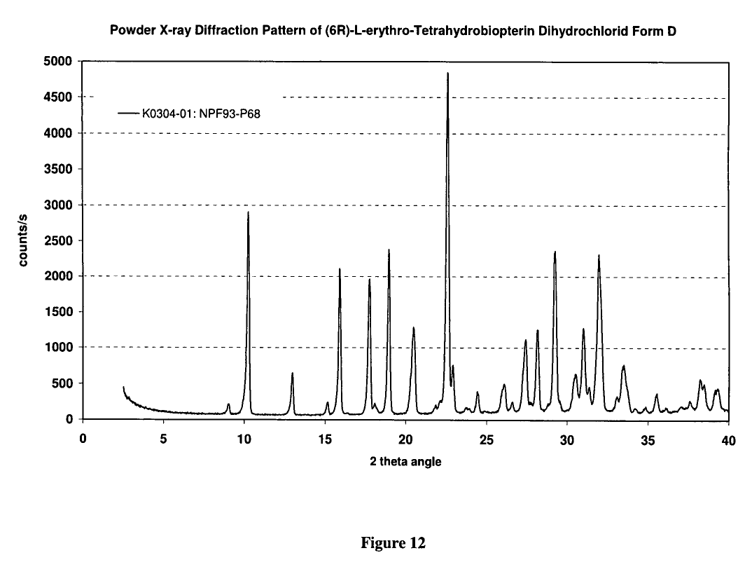

Form D exhibits a characteristic X-ray powder diffraction pattern with characteristic peaks expressed in d-values (Å) at: 8.6 (s), 6.8 (w), 5.56 (m), 4.99 (m), 4.67 (s), 4.32 (m), 3.93 (vs), 3.88 (w), 3.64 (w), 3.41 (w), 3.25 (w), 3.17 (m), 3.05 (s), 2.94 (w), 2.92 (w), 2.88 (m), 2.85 (w), 2.80 (w), 2.79 (m), 2.68 (w), 2.65 (w), 2.52 (vw), 2.35 (w), 2.34 (w), 2.30 (w), and 2.29 (w). FIG. 12 is a graph of the characteristic X-ray diffraction pattern exhibited by hydrate form D of (6R)-L-erythro-tetrahydrobiopterin dihydrochloride.

Hydrate form D may be obtained by adding at about room temperature concentrated aqueous solutions of (6R)-L-erythro-tetrahydrobiopterin dihydrochloride to an excess of a non-solvent such as hexane, heptane, dichloromethane, 1- or 2-propanol, acetone, ethyl acetate, acetonitrile, acetic acid or ethers such as terahydrofuran, dioxane, tertiary-butyl methyl ether, or mixtures of such non-solvents, and stirring the suspension at ambient temperatures. The crystalline solid can be filtered off and then dried under dry nitrogen at ambient temperatures. A preferred non-solvent is isopropanol. The addition of the aqueous solution may carried out drop-wise to avoid a sudden precipitation. Hydrate form D of (6R)-L-erythro-tetrahydrobiopterin dihydrochloride can be prepared by adding at about room temperature a concentrated aqueous solutions of (6R)-L-erythro-tetrahydrobiopterin dihydrochloride to an excess of a non-solvent and stirring the suspension at ambient temperatures. Excess of non-solvent may mean a ratio of aqueous to the non-solvent from 1:10 to 1:1000. Form D contains a small excess of water, related to the monohydrate, and it is believed that it is absorbed water due to the slightly hygroscopic nature of this crystalline hydrate. Hydrate form D is deemed to be the most stable one under the known hydrates at ambient temperatures and a relative humidity of less than 70%. Hydrate form D may be used for formulations prepared under conditions, where this hydrate is stable. Ambient temperature may mean 20 to 30° C.

### Hydrate Form E

It has been found that another hydrate crystal form of (6R)-L-erythro-tetrahydrobiopterin dihydrochloride is a stable preferred form of BH4 for use in a pharmaceutical preparation described herein, which shall be referred to herein as “form E,” or “hydrate E.” The hydrate form E has a water content of approximately 10 to 14 percent by weight, which suggests that form E is a dihydrate. The hydrate E is formed at temperatures below room temperature. Hydrate form E is especially suitable as intermediate and starting material to produce stable polymorph forms. It is especially suitable to produce the water-free form J upon drying under nitrogen or optionally under vacuum. Form E is non-hygroscopic and stable under rather high relative humidities, i.e., at relative humidities above about 60% and up to about 85%. Polymorph form E can be prepared as a solid powder with desired medium particle size range which is typically ranging from 1 μm to about 500 μm.

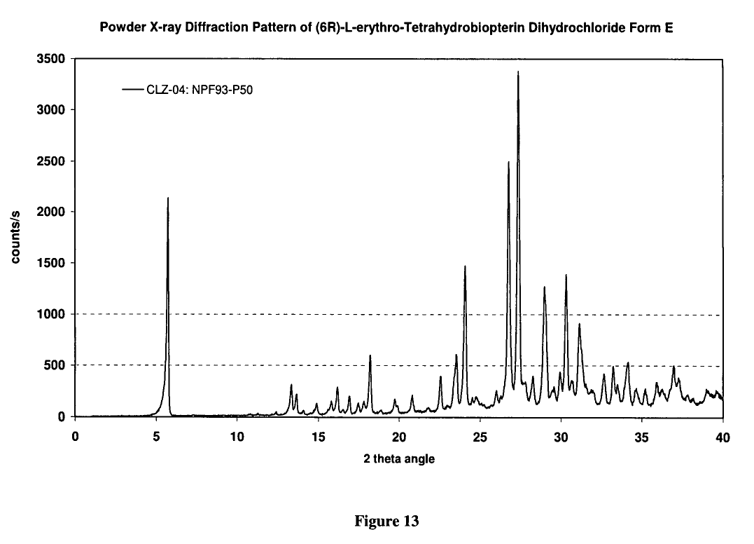

Form E exhibits a characteristic X-ray powder diffraction pattern with characteristic peaks expressed in d-values (Å) at: 15.4 (s), 6.6 (w), 6.5 (w), 5.95 (vw), 5.61 (vw), 5.48 (w), 5.24 (w), 4.87 (w), 4.50 (vw), 4.27 (w), 3.94 (w), 3.78 (w), 3.69 (m), 3.60 (w), 3.33 (s), 3.26 (vs), 3.16 (w), 3.08 (m), 2.98 (w), 2.95 (m), 2.91 (w), 2.87 (m), 2.79 (w), 2.74 (w), 2.69 (w), and 2.62 (w). FIG. 13 is a graph of the characteristic X-ray diffraction pattern exhibited by hydrate form E of (6R)-L-erythro-tetrahydrobiopterin dihydrochloride.

Hydrate form E may be obtained by adding concentrated aqueous solutions of (6R)-L-erythro-tetrahydrobiopterin dihydrochloride to an excess of a non-solvent cooled to temperatures from about 10 to −10° C. and preferably between 0 to 10° C. and stirring the suspension at said temperatures. The crystalline solid can be filtered off and then dried under dry nitrogen at ambient temperatures. Non-solvents are for example such as hexane, heptane, dichloromethane, 1- or 2-propanol, acetone, ethyl acetate, acetonitrile, acetic acid or ethers such as terahydrofuran, dioxane, tertiary-butyl methyl ether, or mixtures of such non-solvents. A preferred non-solvent is isopropanol. The addition of the aqueous solution may carried out drop-wise to avoid a sudden precipitation. Hydrate form E of (6R)-L-erythro-tetrahydrobiopterin dihydrochloride can be prepared by adding a concentrated aqueous solutions of (6R)-L-erythro-tetrahydrobiopterin dihydrochloride to an excess of a non-solvent, which is cooled to temperatures from about 10 to −10° C., and stirring the suspension at ambient temperatures. Excess of non-solvent may mean a ratio of aqueous to the non-solvent from 1:10 to 1:1000. A preferred non-solvent is tetrahydrofuran. Another preparation process comprises exposing polymorph form B to an air atmosphere with a relative humidity of 70 to 90%, preferably about 80%. Hydrate form E is deemed to be a dihydrate, whereby some additional water may be absorbed. Polymorph form E can be transformed into polymorph J upon drying under vacuum at moderate temperatures, which may mean between 20° C. and 50° C. at pressures between 0 and 100 mbar. Form E is especially suitable for formulations in semi solid forms because of its stability at high relative humidities.

### Hydrate Form H

It has been found that another hydrate crystal form of (6R)-L-erythro-tetrahydrobiopterin dihydrochloride is a stable preferred form of BH4 for use in a pharmaceutical preparation described herein, which shall be referred to herein as “form H,” or “hydrate H.” The hydrate form H has a water content of approximately 5.0 to 7.0 percent by weight, which suggests that form H is a hygroscopic monohydrate. The hydrate form H is formed at temperatures below room temperature. Hydrate form H is especially suitable as intermediate and starting material to produce stable polymorph forms. Polymorph form H can be prepared as a solid powder with desired medium particle size range which is typically ranging from 1 μm to about 500 μm.

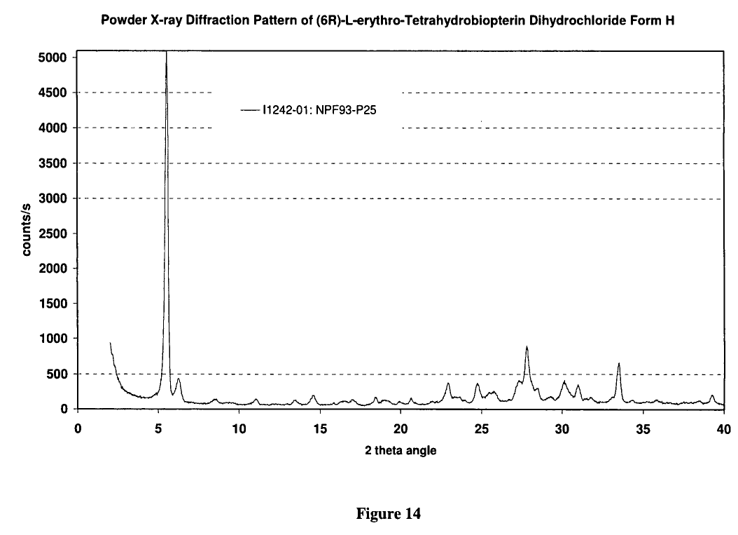

Form H exhibits a characteristic X-ray powder diffraction pattern with characteristic peaks expressed in d-values (Å) at: 8.6 15.8 (vs), 10.3 (w), 8.0 (w), 6.6 (w), 6.07 (w), 4.81 (w), 4.30 (w), 3.87 (m), 3.60 (m), 3.27 (m), 3.21 (m), 3.13 (w), 3.05 (w), 2.96 (m), 2.89 (m), 2.82 (w), and 2.67 (m). FIG. 14 is a graph of the characteristic X-ray diffraction pattern exhibited by hydrate form H of (6R)-L-erythro-tetrahydrobiopterin dihydrochloride.

Hydrate form H may be obtained by dissolving at ambient temperatures (6R)-L-erythro-tetrahydrobiopterin dihydrochloride in a mixture of acetic acid and water, adding then a non-solvent to precipitate a crystalline solid, cooling the obtained suspension and stirring the cooled suspension for a certain time. The crystalline solid is filtered off and then dried under vacuum at ambient temperatures. Non-solvents are for example such as hexane, heptane, dichloromethane, 1- or 2-propanol, acetone, ethyl acetate, acetonitril, acetic acid or ethers such as terahydrofuran, dioxane, tertiary-butyl methyl ether, or mixtures of such non-solvents. A preferred non-solvent is tetrahydrofuran. Hydrate form H of (6R)-L-erythro-tetrahydrobiopterin dihydrochloride can be by prepared by dissolving at ambient temperatures (6R)-L-erythro-tetrahydrobiopterin dihydrochloride in a mixture of acetic acid and a less amount than that of acetic acid of water, adding a non-solvent and cooling the obtained suspension to temperatures in the range of −10 to 10° C., and preferably −5 to 5° C., and stirring the suspension at said temperature for a certain time. Certain time may mean 1 to 20 hours. The weight ratio of acetic acid to water may be from 2:1 to 25:1 and preferably 5:1 to 15:1. The weight ratio of acetic acid/water to the non-solvent may be from 1:2 to 1:5. Hydrate form H seems to be a monohydrate with a slight excess of water absorbed due to the hygroscopic nature.

### Hydrate Form O

It has been found that another hydrate crystal form of (6R)-L-erythro-tetrahydrobiopterin dihydrochloride is a stable preferred form of BH4 for use in a pharmaceutical preparation described herein, which shall be referred to herein as “form O,” or “hydrate O.” The hydrate form O is formed at temperatures near room temperature. Hydrate form O is especially suitable as intermediate and starting material to produce stable polymorph forms. Polymorph form O can be prepared as a solid powder with desired medium particle size range which is typically ranging from 1 μm to about 500 μm.

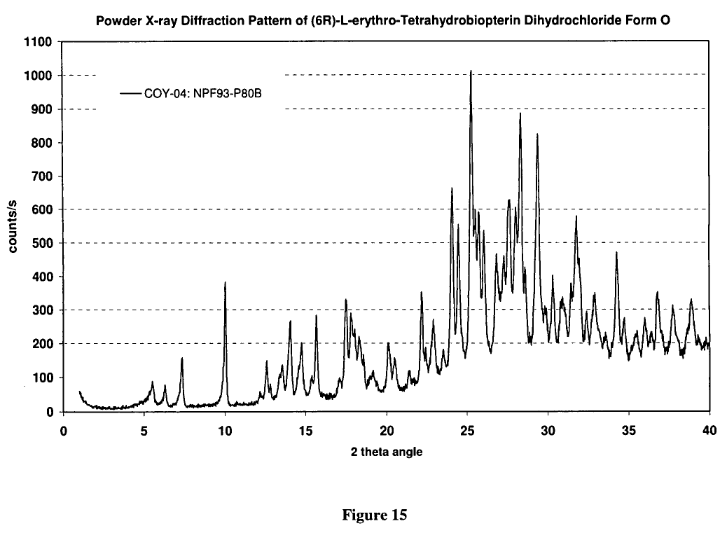

Form O exhibits a characteristic X-ray powder diffraction pattern with characteristic peaks expressed in d-values (Å) at: 15.9 (w), 14.0 (w), 12.0 (w), 8.8 (m), 7.0 (w), 6.5 (w), 6.3 (m), 6.00 (w), 5.75 (w), 5.65 (m), 5.06 (m), 4.98 (m), 4.92 (m), 4.84 (w), 4.77 (w), 4.42 (w), 4.33 (w), 4.00 (m), 3.88 (m), 3.78 (w), 3.69 (s), 3.64 (s), 3.52 (vs), 3.49 (s), 3.46 (s), 3.42 (s), 3.32 (m), 3.27 (m), 3.23 (s), 3.18 (s), 3.15 (vs), 3.12 (m), 3.04 (vs), 2.95 (m), 2.81 (s), 2.72 (m), 2.67 (m), and 2.61 (m). FIG. 15 is a graph of the characteristic X-ray diffraction pattern exhibited by hydrate form O of (6R)-L-erythro-tetrahydrobiopterin dihydrochloride.

Hydrate form O can be prepared by exposure of polymorphic form F to a nitrogen atmosphere containing water vapor with a resulting relative humidity of about 52% for about 24 hours. The fact that form F, which is a slightly hygroscopic anhydrate, can be used to prepare form O under 52% relative humidity suggests that form O is a hydrate, which is more stable than form F under ambient temperature and humidity conditions.

Solvate Forms of (6R) L-Tetrahydrobiopterin Dihydrochloride Salt

As further described below, it has been found that (6R)-L-erythro-tetrahydrobiopterin dihydrochloride exists as a number of crystalline solvate forms, which shall be described and defined herein as forms G, I, L, M, and N. These solvate forms are useful as a stable form of BH4 for the pharmaceutical preparations described herein and in the preparation of compositions including stable crystal polymorphs of BH4.

### Solvate Form G

It has been found that an ethanol solvate crystal form of (6R)-L-erythro-tetrahydrobiopterin dihydrochloride is a stable preferred form of BH4 for use in a pharmaceutical preparation described herein, which shall be referred to herein as “form G,” or “hydrate G.” The ethanol solvate form G has a ethanol content of approximately 8.0 to 12.5 percent by weight, which suggests that form G is a hygroscopic mono ethanol solvate. The solvate form G is formed at temperatures below room temperature. Form G is especially suitable as intermediate and starting material to produce stable polymorph forms. Polymorph form G can be prepared as a solid powder with a desired medium particle size range which is typically ranging from 1 μm to about 500 μm.

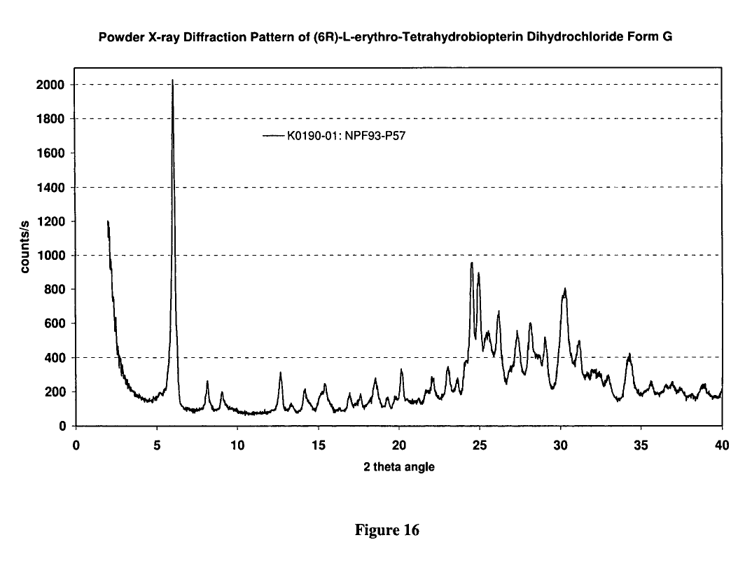

Form G exhibits a characteristic X-ray powder diffraction pattern with characteristic peaks expressed in d-values (Å) at: 14.5 (vs), 10.9 (w), 9.8 (w), 7.0 (w), 6.3 (w), 5.74 (w), 5.24 (vw), 5.04 (vw), 4.79 (w), 4.41 (w), 4.02 (w), 3.86 (w), 3.77 (w), 3.69 (w), 3.63 (m), 3.57 (m), 3.49 (m), 3.41 (m), 3.26 (m), 3.17 (m), 3.07 (m), 2.97 (m), 2.95 (m), 2.87 (w), and 2.61 (w). FIG. 16 is a graph of the characteristic X-ray diffraction pattern exhibited by solvate form G of (6R)-L-erythro-tetrahydrobiopterin dihydrochloride.

Ethanol solvate form G may be obtained by crystallization of L-erythro-tetrahydrobiopterin dihydrochloride dissolved in water and adding a large excess of ethanol, stirring the obtained suspension at or below ambient temperatures and drying the isolated solid under air or nitrogen at about room temperature. Here, a large excess of ethanol means a resulting mixture of ethanol and water with less than 10% water, preferably about 3 to 6%. Ethanolate form G of (6R)-L-erythro-tetrahydrobiopterin dihydrochloride can be prepared by dissolving at about room temperature to temperatures of 75° C. (6R)-L-erythro-tetrahydrobiopterin dihydrochloride in water or in a mixture of water and ethanol, cooling a heated solution to room temperature and down to 5 to 10° C., adding optionally ethanol to complete precipitation, stirring the obtained suspension at temperatures of 20 to 5° C., filtering off the white, crystalline solid and drying the solid under air or a protection gas such as nitrogen at temperatures about room temperature. The process may be carried out in a first variant in dissolving (6R)-L-erythro-tetrahydrobiopterin dihydrochloride at about room temperature in a lower amount of water and then adding an excess of ethanol and then stirring the obtained suspension for a time sufficient for phase equilibration. In a second variant, (6R)-L-erythro-tetrahydrobiopterin dihydrochloride may be suspended in ethanol, optionally adding a lower amount of water, and heating the suspension and dissolute (6R)-L-erythro-tetrahydrobiopterin dihydrochloride, cooling down the solution to temperatures of about 5 to 15° C., adding additional ethanol to the suspension and then stirring the obtained suspension for a time sufficient for phase equilibration.

### Solvate Form I

It has been found that an acetic acid solvate crystal form of (6R)-L-erythro-tetrahydrobiopterin dihydrochloride is a stable preferred form of BH4 for use in a pharmaceutical preparation described herein, which shall be referred to herein as “form I,” or “hydrate I.” The acetic acid solvate form I has an acetic acid content of approximately 12.7 percent by weight, which suggests that form I is a hygroscopic acetic acid mono solvate. The solvate form I is formed at temperatures below room temperature. Acetic acid solvate form I is especially suitable as intermediate and starting material to produce stable polymorph forms. Polymorph form I can be prepared as a solid powder with desired medium particle size range which is typically ranging from 1 μm to about 500 μm.

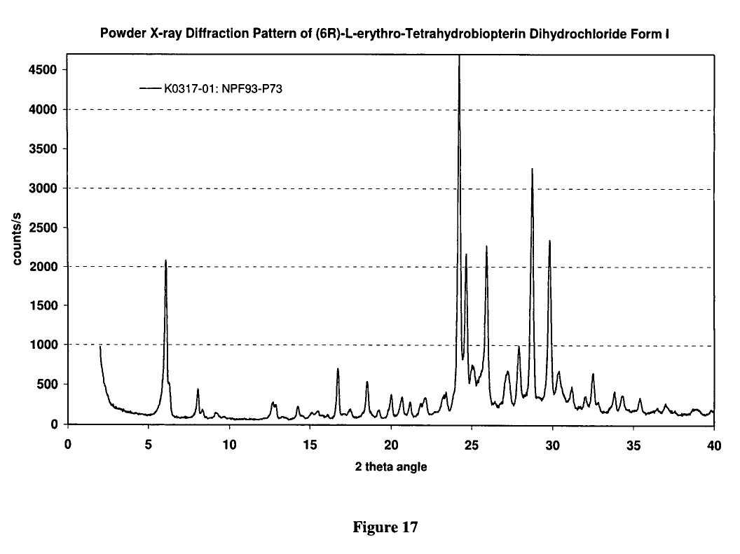

Form I exhibits a characteristic X-ray powder diffraction pattern with characteristic peaks expressed in d-values (Å) at: 14.5 (m), 14.0 (w), 11.0 (w), 7.0 (vw), 6.9 (vw), 6.2 (vw), 5.30 (w), 4.79 (w), 4.44 (w), 4.29 (w), 4.20 (vw), 4.02 (w), 3.84 (w), 3.80 (w), 3.67 (vs), 3.61 (m), 3.56 (w), 3.44 (m), 3.27 (w), 3.19 (w), 3.11(s), 3.00 (m), 2.94 (w), 2.87 (w), and 2.80 (w). FIG. 17 is a graph of the characteristic X-ray diffraction pattern exhibited by solvate form I of (6R)-L-erythro-tetrahydrobiopterin dihydrochloride.

Acetic acid solvate form I may be obtained by dissolution of L-erythro-tetrahydrobiopterin dihydrochloride in a mixture of acetic acid and water at elevated temperature, adding further acetic acid to the solution, cooling down to a temperature of about 10° C., then warming up the formed suspension to about 15° C., and then stirring the obtained suspension for a time sufficient for phase equilibration, which may last up to 3 days. The crystalline solid is then filtered off and dried under air or a protection gas such as nitrogen at temperatures about room temperature.

### Solvate Form L

It has been found that a mixed ethanol solvate/hydrate crystal form of (6R)-L-erythro-tetrahydrobiopterin dihydrochloride is a stable preferred form of BH4 for use in a pharmaceutical preparation described herein, which shall be referred to herein as “form L,” or “hydrate L.” Form L may contain 4% but up to 13% ethanol and 0% to about 6% of water. Form L may be transformed into form G when treated in ethanol at temperatures from about 0° C. to 20° C. In addition form L may be transformed into form B when treated in an organic solvent at ambient temperatures (10° C. to 60° C.). Polymorph form L can be prepared as a solid powder with desired medium particle size range which is typically ranging from 1 μm to about 500 μm.

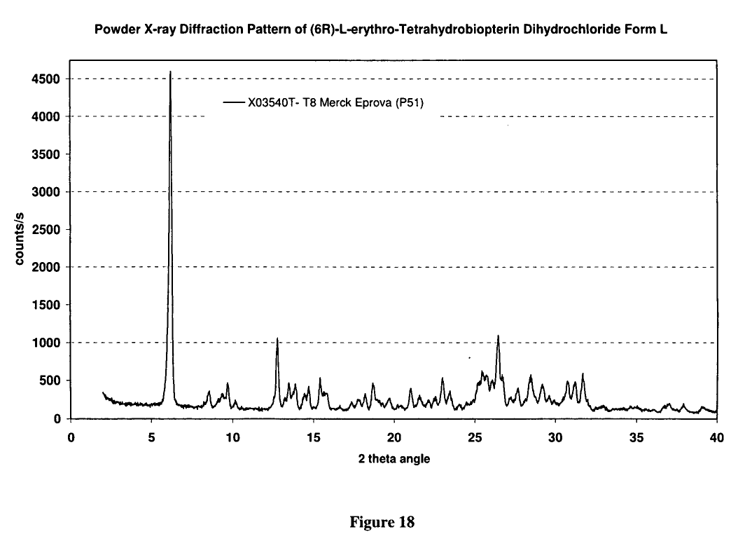

Form L exhibits a characteristic X-ray powder diffraction pattern with characteristic peaks expressed in d-values (Å) at: 14.1 (vs), 10.4 (w), 9.5 (w), 9.0 (vw), 6.9 (w), 6.5 (w), 6.1 (w), 5.75 (w), 5.61 (w), 5.08 (w), 4.71 (w), 3.86 (w), 3.78 (w), 3.46 (m), 3.36 (m), 3.06 (w), 2.90 (w), and 2.82 (w). FIG. 18 is a graph of the characteristic X-ray diffraction pattern exhibited by solvate form L of (6R)-L-erythro-tetrahydrobiopterin dihydrochloride.

Form L may be obtained by suspending hydrate form E at room temperature in ethanol and stirring the suspension at temperatures from 0 to 10° C., preferably about 5° C., for a time sufficient for phase equilibration, which may be 10 to 20 hours. The crystalline solid is then filtered off and dried preferably under reduced pressure at 30° C. or under nitrogen. Analysis by TG-FTIR suggests that form L may contain variable amounts of ethanol and water, i.e., it can exist as an polymorph (anhydrate), as a mixed ethanol solvate/hydrate, or even as a hydrate.

### Solvate Form M

It has been found that an ethanol solvate crystal form of (6R)-L-erythro-tetrahydrobiopterin dihydrochloride is a stable preferred form of BH4 for use in a pharmaceutical preparation described herein, which shall be referred to herein as “form M,” or “hydrate M.” Form M may contain 4% but up to 13% ethanol and 0% to about 6% of water, which suggests that form M is a slightly hygroscopic ethanol solvate. The solvate form M is formed at room temperature. Form M is especially suitable as intermediate and starting material to produce stable polymorph forms, since form M can be transformed into form G when treated in ethanol at temperatures between about −10° to 15° C., and into form B when treated in organic solvents such as ethanol, C3 and C4 alcohols, or cyclic ethers such as THF and dioxane. Polymorph form M can be prepared as a solid powder with desired medium particle size range which is typically ranging from 1 μm to about 500 μm.

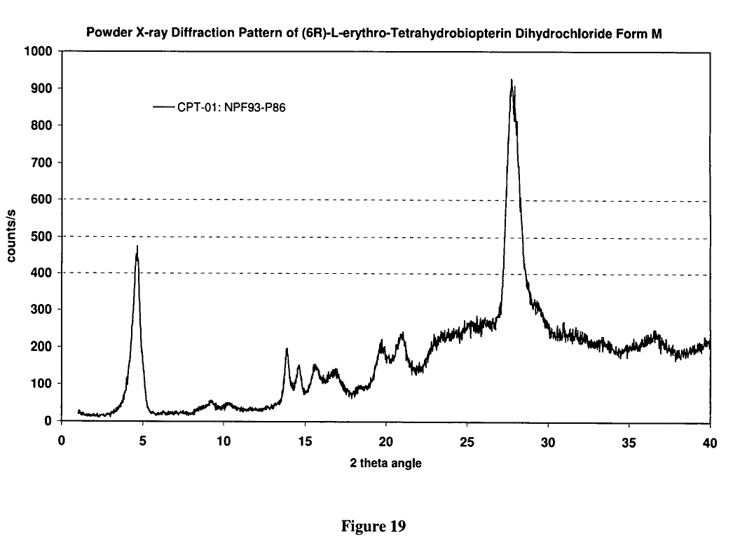

Form M exhibits a characteristic X-ray powder diffraction pattern with characteristic peaks expressed in d-values (Å) at: 18.9 (s), 6.4 (m), 6.06 (w), 5.66 (w), 5.28 (w), 4.50 (w), 4.23 (w), and 3.22 (vs). FIG. 19 is a graph of the characteristic X-ray diffraction pattern exhibited by solvate form M of (6R)-L-erythro-tetrahydrobiopterin dihydrochloride.

Ethanol solvate form M may be obtained by dissolution of L-erythro-tetrahydrobiopterin dihydrochloride in ethanol and evaporation of the solution under nitrogen at ambient temperature, i.e., between 10° C. and 40° C. Form M may also be obtained by drying of form G under a slight flow of dry nitrogen at a rate of about 20 to 100 ml/min. Depending on the extent of drying under nitrogen, the remaining amount of ethanol may be variable, i.e., from about 3% to 13%.

### Solvate Form N

It has been found that another solvate crystal form of (6R)-L-erythro-tetrahydrobiopterin dihydrochloride is a stable preferred form of BH4 for use in a pharmaceutical preparation described herein, which shall be referred to herein as “form N,” or “hydrate N.” Form N may contain in total up to 10% of isopropanol and water, which suggests that form N is a slightly hygroscopic isopropanol solvate. Form N may be obtained through washing of form D with isopropanol and subsequent drying in vacuum at about 30° C. Form N is especially suitable as intermediate and starting material to produce stable polymorph forms. Polymorph form N can be prepared as a solid powder with desired medium particle size range which is typically ranging from 1 μm to about 500 μm.

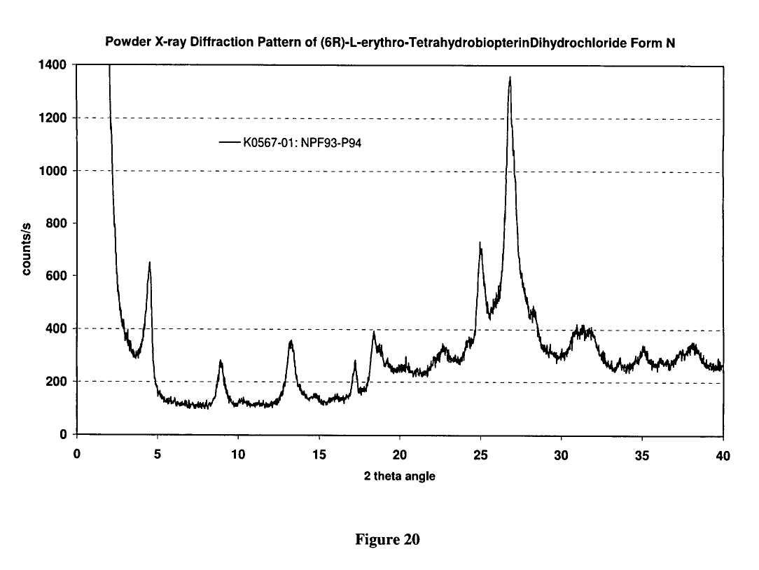

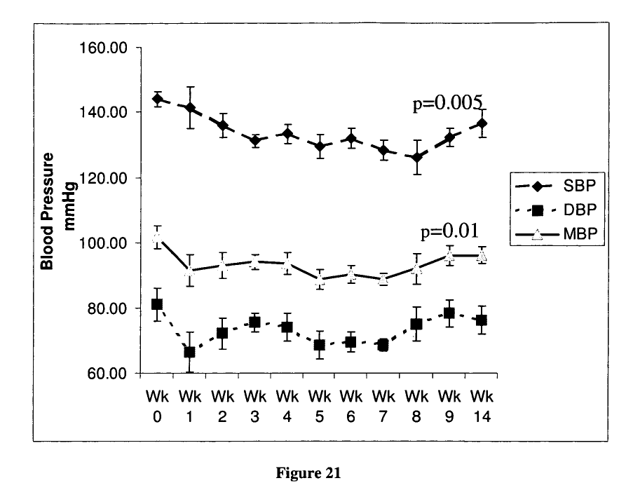

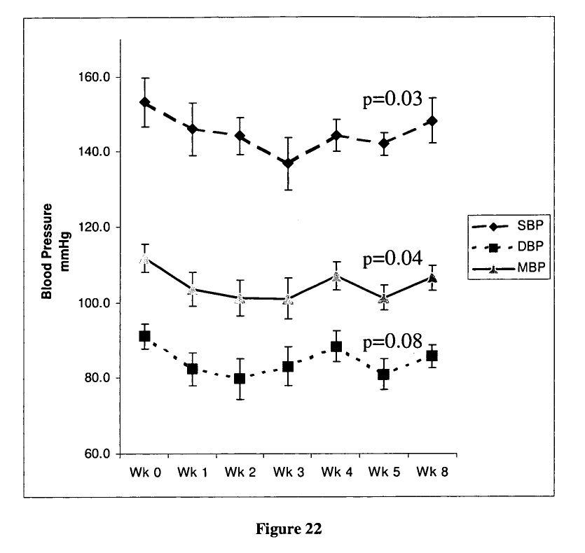

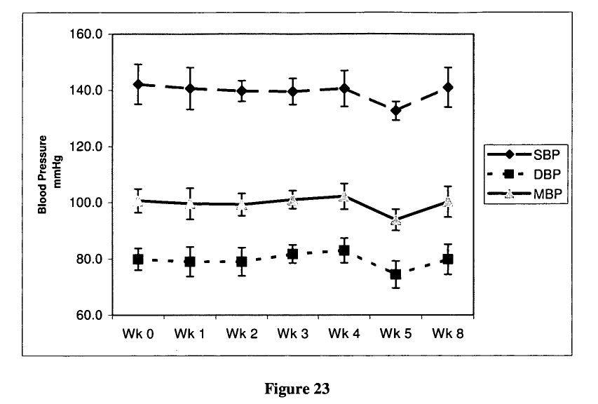

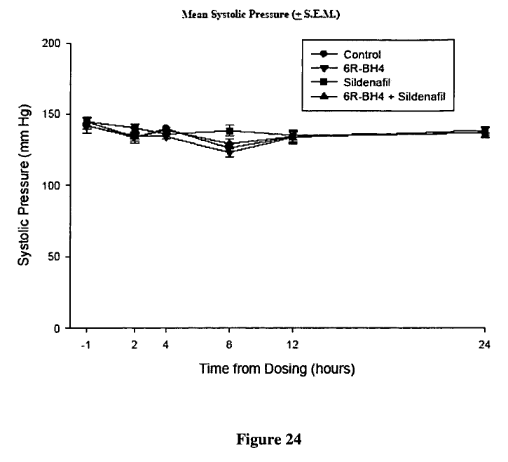

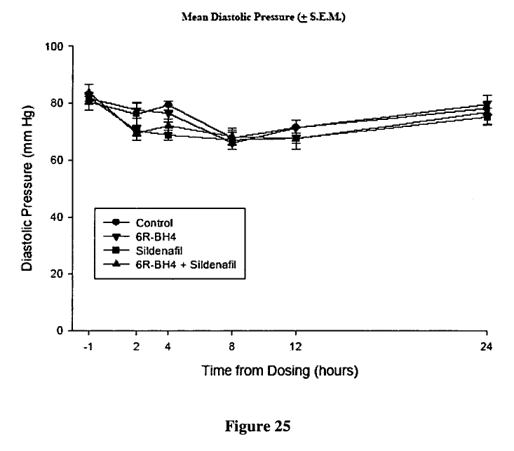

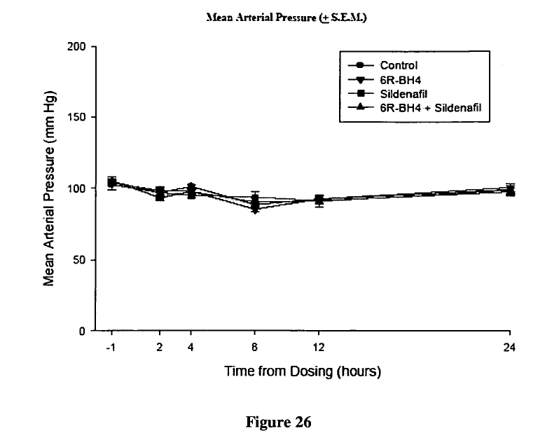

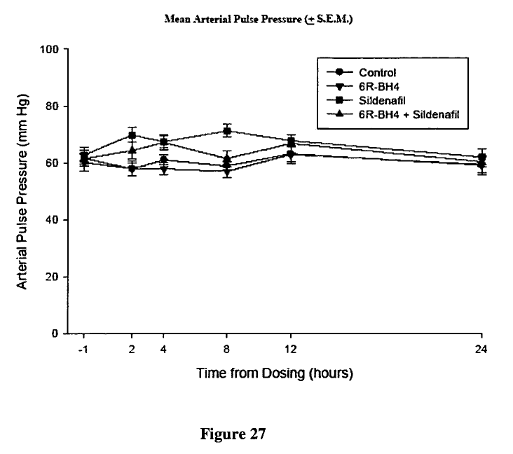

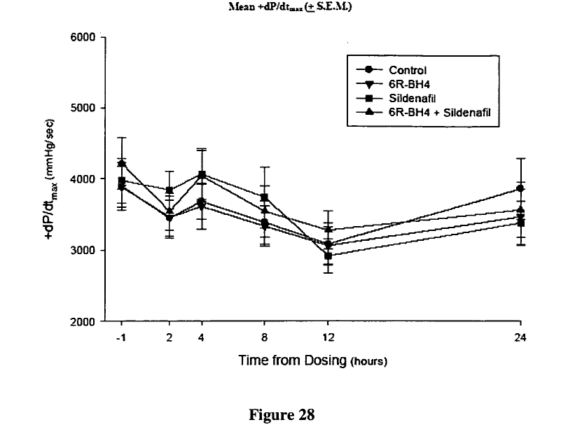

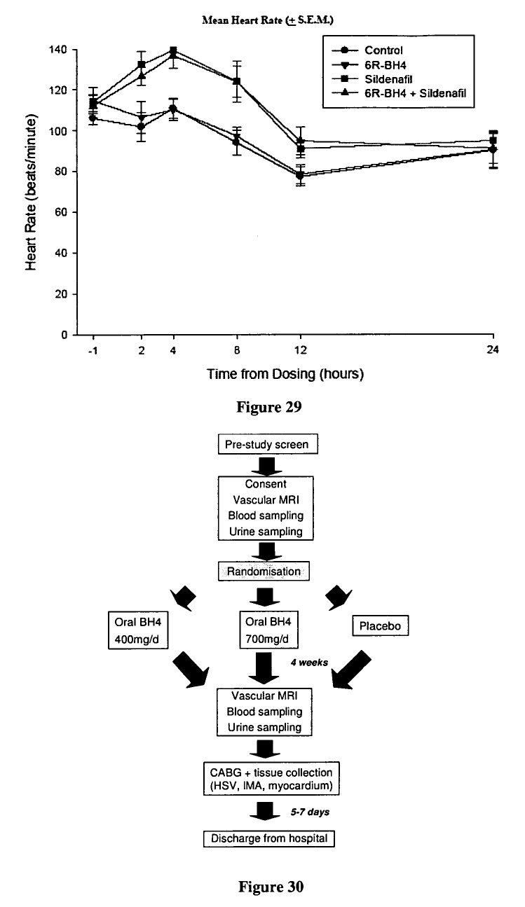

Form N exhibits a characteristic X-ray powder diffraction pattern with characteristic peaks expressed in d-values (Å) at: 19.5 (m), 9.9 (w), 6.7 (w), 5.15 (w), 4.83(w), 3.91 (w), 3.56 (m), 3.33 (vs), 3.15 (w), 2.89 (w), 2.81 (w), 2.56 (w), and 2.36 (w). FIG. 20 is a graph of the characteristic X-ray diffraction pattern exhibited by solvate form N of (6R)-L-erythro-tetrahydrobiopterin dihydrochloride.

The isopropanol form N may be obtained by dissolution of L-erythro-tetrahydrobiopterin dihydrochloride in 4.0 ml of a mixture of isopropanol and water (mixing volume ratio for example 4:1). To this solution is slowly added isopropanol (IPA, for example about 4.0 ml) and the resulting suspension is cooled to 0° C. and stirred for several hours (e.g., about 10 to 18 hours) at this temperature. The suspension is filtered and the solid residue washed with isopropanol at room temperature. The obtained crystalline material is then dried at ambient temperature (e.g., about 20 to 30° C.) and reduced pressure (about 2 to 10 mbar) for several hours (e.g., about 5 to 20 hours). TG-FTIR shows a weight loss of 9.0% between 25 to 200° C., which is attributed to both isopropanol and water. This result suggests that form N can exist either in form of an isopropanol solvate, or in form of mixed isopropanol solvate/hydrate, or as an non-solvated form containing a small amount of water.

For the preparation of the polymorph forms, there may be used crystallization techniques well known in the art, such as stirring of a suspension (phase equilibration in), precipitation, re-crystallization, evaporation, solvent like water sorption methods or decomposition of solvates. Diluted, saturated or super-saturated solutions may be used for crystallization, with or without seeding with suitable nucleating agents. Temperatures up to 100° C. may be applied to form solutions. Cooling to initiate crystallization and precipitation down to −100° C. and preferably down to −30° C. may be applied. Meta-stable polymorphs or pseudo-polymorphic forms can be used to prepare solutions or suspensions for the preparation of more stable forms and to achieve higher concentrations in the solutions.

It was surprisingly found that hydrate form D is the most stable form under the hydrates and forms B and D are especially suitable to be used in pharmaceutical formulations. Forms B and D presents some advantages like an aimed manufacture, good handling due to convenient crystal size and morphology, very good stability under production conditions of various types of formulation, storage stability, higher solubility, and high bioavailability. Accordingly, one embodiment of the compositions and methods disclosed herein is pharmaceutical composition including polymorph form B and/or hydrate form D of (6R)-L-erythro-tetrahydrobiopterin dihydrochloride and a pharmaceutically acceptable carrier or diluent.

The crystal forms of (6R)-L-erythro-tetrahydrobiopterin dihydrochloride may be used together with folic acid or tetrahydrofolic acid or their pharmaceutically acceptable salts such as sodium, potassium, calcium or ammonium salts, each alone or additionally with arginine. The weight ratio of crystal forms: folic acids or salts thereof: arginine may be from about 1:10:10 to about 10:1:1.

The invention provides methods of using any of the tetrahydrobiopterin polymorphs described herein, or stable pharmaceutical preparations comprising any of such polymorphs, for treatment of conditions associated with endothelial dysfunction. Concurrent treatment with folates, including folate precursors, folic acids, or folate derivatives, is also contemplated, as is treatment with a pharmaceutical composition or foodstuff that comprises both a tetrahydrobiopterin, or a BH4 precursor or BH4 derivative, and a folate. Exemplary folates are disclosed in U.S. Pat. Nos. 6,011,040 and 6,544,994, both of which are incorporated herein by reference, and include folic acid (pteroylmonoglutamate), dihydrofolic acid, tetrahydrofolic acid, 5-methyltetrahydrofolic acid, 5,10-methylenetetrahydrofolic acid, 5,10-methenyltetrahydrofolic acid, 5,10-formiminotetrahydrofolic acid, 5-formyltetrahydrofolic acid (leucovorin), 10-formyltetrahydrofolic acid, 10-methyltetrahydrofolic acid, one or more of the folylpolyglutamates, compounds in which the pyrazine ring of the pterin moiety of folic acid or of the folylpolyglutamates is reduced to give dihydrofolates or tetrahydrofolates, or derivatives of all the preceding compounds in which the N-5 or N-10 positions carry one carbon units at various levels of oxidation, or pharmaceutically compatible salts thereof, or a combination of two or more thereof. Exemplary tetrahydrofolates include 5-formyl-(6S)-tetrahydrofolic acid, 5-methyl-(6S)-tetrahydrofolic acid, 5,10-methylene-(6R)-tetrahydrofolic acid, 5,10-methenyl-(6R)-tetrahydrofolic acid, 10-formyl-(6R)-tetrahydrofolic acid, 5-formimino-(6S)-tetrahydrofolic acid or (6S)-tetrahydrofolic acid, and salts thereof.

### 4. Pharmaceutical Formulations

The formulations described herein are preferably administered as oral formulations. Oral formulations are preferably solid formulations such as capsules, tablets, pills and troches, or liquid formulations such as aqueous suspensions, elixirs and syrups. The various form of BH4 described herein can be directly used as powder (micronized particles), granules, suspensions or solutions, or it may be combined together with other pharmaceutically acceptable ingredients in admixing the components and optionally finely divide them, and then filling capsules, composed for example from hard or soft gelatin, compressing tablets, pills or troches, or suspend or dissolve them in carriers for suspensions, elixirs and syrups. Coatings may be applied after compression to form pills.

Pharmaceutically acceptable ingredients are well known for the various types of formulation and may be for example binders such as natural or synthetic polymers, excipients, lubricants, surfactants, sweetening and flavoring agents, coating materials, preservatives, dyes, thickeners, adjuvants, antimicrobial agents, antioxidants and carriers for the various formulation types. Nonlimiting examples of binders useful in a composition described herein include gum tragacanth, acacia, starch, gelatin, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, und natural polymers like chitosan.

Nonlimiting examples of excipients useful in a composition described herein include phosphates such as dicalcium phosphate. Nonlimiting examples of lubricants use in a composition described herein include natural or synthetic oils, fats, waxes, or fatty acid salts such as magnesium stearate.

Surfactants for use in a composition described herein can be anionic, anionic, amphoteric or neutral. Nonlimiting examples of surfactants useful in a composition described herein include lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Nonlimiting examples of sweetening agents useful in a composition described herein include sucrose, fructose, lactose or aspartame. Nonlimiting examples of flavoring agents for use in a composition described herein include peppermint, oil of wintergreen or fruit flavors such as cherry or orange flavor. Nonlimiting examples of coating materials for use in a composition described herein include gelatin, wax, shellac, sugar or other biological degradable polymers. Nonlimiting examples of preservatives for use in a composition described herein include methyl or propylparabens, sorbic acid, chlorobutanol, phenol and thimerosal.

The hydrate form D described herein may also be formulated as effervescent tablet or powder, which disintegrate in an aqueous environment to provide a drinking solution. A syrup or elixir may contain the polymorph described herein, sucrose or fructose as sweetening agent a preservative like methylparaben, a dye and a flavoring agent.

Slow release formulations may also be prepared from the polymorph described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

While individual needs vary, determination of optimal ranges of effective amounts of each component is within the skill of the art. Typical dosages of the BH4 comprise about 1 to about 20 mg/kg body weight per day, which will usually amount to about 5 (1 mg/kg×5 kg body weight) to 3000 mg/day (30 mg/kg×100 kg body weight). Such a dose may be administered in a single dose or it may be divided into multiple doses. While continuous, daily administration is contemplated, it may be desirable to ceases the BH4 therapy when specific clinical indicators are improved to above a certain threshold level. Of course, the therapy may be reinitiated in the event that clinical improvement indicators deteriorate.

It is understood that the suitable dose of a composition according to the present invention will depend upon the age, health and weight of the recipient, kind of concurrent treatment, if any, frequency of treatment, and the nature of the effect desired (i.e., the amount of decrease in pulmonary pressures desired). The frequency of dosing also is dependent on pharmacodynamic effects on arterial oxygen pressures. However, the most preferred dosage can be tailored to the individual subject, as is understood and determinable by one of skill in the art, without undue experimentation. This typically involves adjustment of a standard dose, e.g., reduction of the dose if the patient has a low body weight.

As discussed above, the total dose required for each treatment may be administered in multiple doses or in a single dose. The BH4 compositions may be administered alone or in conjunction with other therapeutics directed to the disease or directed to other symptoms thereof.

As is apparent from the disclosure presented herein, in a broad aspect the present application contemplates clinical application of a composition that contains a crystallized BH4 formulation. The compositions should be formulated into suitable pharmaceutical compositions, i.e., in a form appropriate for in vivo applications in such combination therapies. Generally, this will entail preparing compositions that are essentially free of pyrogens, as well as other impurities that could be harmful to humans or animals. Preferably, the formulation comprising the crystallized BH4 composition may be such that it can be used directly for the treatment of vascular disease.

One will generally desire to employ appropriate salts and buffers to render the BH4 suitable for uptake. Aqueous compositions of the present invention comprise an effective amount of the BH4 dissolved or dispersed in a pharmaceutically acceptable carrier or aqueous medium. Such compositions may be administered orally or via injection.

The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also can be incorporated into the compositions. In exemplary embodiments, the medical protein formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and cyanocobalamin. The amino acids, minerals and vitamins in the supplement should be provided in amounts that provide the recommended daily doses of each of the components.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The active compositions of the present invention include classic pharmaceutical preparations of BH4, which have been discussed herein as well as those known to those of skill in the art. Administration of these compositions according to the present invention will be via any common route for dietary supplementation. The protein is preferably administered orally, as is the BH4.

In certain embodiments, it is contemplated that BH4 or precursors or derivatives thereof used for the treatment of vascular diseases are formulated as an inhalable formulation for administration through inhalation. As such, the BH4 or precursors or derivatives thereof may be prepared as an aerosol formulation. Methods to the treatment of pulmonary hypertension using inhalable compositions are known to those of skill in the art and are described, for example, in U.S. Pat. No. 6,756,033 (incorporated herein by reference), which provides a teaching of treatment of pulmonary hypertension by delivering prostaglandin preparations by inhalation. The inhalation techniques described in the aforementioned patent for prostaglandins also will be useful in producing inhalable preparations of BH4 and/or its precursors and derivatives. In addition, it is contemplated that endothelial dysfunction may be treated by a combined administration of BH4-based compositions and prostaglandin preparations.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The BH4 compositions may be prepared as pharmaceutical forms suitable for injectable use. Such compositions include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

A preferred formulation for the compositions of BH4 and for use with the methods described herein is a tablet formulation. It has surprisingly been found that the addition of ascorbic acid to a tablet formulation increase the stability of the formulation. Without intending to be limited to a particular mechanism of stabilization, it is believed that when the BH4 is mixed into a pharmaceutical formulation with a variety of excipients that the even a small amount of ascorbic acid (e.g., less than 2% by weight) creates a complex with the BH4 and inhibits one or more pathways in which the BH4 is degraded. Thus, as set forth in greater detail in Int'l Application No. PCT/US05/41252 filed Nov. 16, 2005, published as WO 2006/055511, incorporated herein by reference in its entirety, one exemplary tablet formulation of BH4 for use herein includes ascorbic acid.

Exemplary stable oral formulations contain one or more of the following additional ingredients that improve the stability or other characteristics of the formulation: binder, disintegration agent, acidic antioxidant, or lubricant or combinations thereof. Exemplary stable tablet formulations include a binder and disintegration agent, optionally with an acidic antioxidant, and optionally further including a lubricant. Exemplary concentrations of binder are between about 1 wt % to about 5 wt %, or between about 1.5 and 3 wt %; an exemplary weight ratio of binder to BH4 is in the range of about 1:10 to about 1:20. Exemplary concentrations of disintegration agent are between about 1 wt % to about 20 wt %; an exemplary weight ratio of disintegration agent to BH4 is in the range of about 1:5 to about 1:10. Exemplary concentrations of antioxidant are between about 1 wt % and about 3 wt %; an exemplary weight ratio of antioxidant to BH4 is in the range of about 1:5 to 1:30. In one example, ascorbic acid is the antioxidant and is used at a ratio to BH4 of less than 1:1, e.g. 1:2 or less, or 1:10 or less. Exemplary concentrations of lubricant in a stable tablet formulation of the present invention are between about 0.1 wt % and about 2 wt %; an exemplary weight ratio of lubricant to BH4 is in the range of about 1:25 to 1:65.

The stable solid formulation may optionally include other therapeutic agents suitable for the condition to be treated, e.g. folates, including folate precursors, folic acids, or folate derivatives; and/or arginine; and/or vitamins, such as vitamin C and/or vitamin B2 (riboflavin) and/or vitamin B12; and/or neurotransmitter precursors such as L-dopa or carbidopa; and/or 5-hydroxytryptophan.

The BH4 used in a composition described herein is preferably formulated as a dihydrochloride salt, however, it is contemplated that other salt forms of BH4 posses the desired biological activity, and consequently, other salt forms of BH4 can be used.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′ dibenzylethylenediamine, chloroprocaine, choline, diethanolamine, dicyclohexylamine, ethylenediamine, N methylglucamine, and procaine.

Pharmaceutically acceptable acid addition salts include inorganic or organic acid salts. Examples of suitable acid salts include the hydrochlorides, acetates, citrates, salicylates, nitrates, phosphates. Other suitable pharmaceutically acceptable salts are well known to those skilled in the art and include, for example, acetic, citric, oxalic, tartaric, or mandelic acids, hydrochloric acid, hydrobromic acid, sulfuric acid or phosphoric acid; with organic carboxylic, sulfonic, sulfo or phospho acids or N substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4 aminosalicylic acid, 2 phenoxybenzoic acid, 2 acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2 hydroxyethanesulfonic acid, ethane 1,2 disulfonic acid, benzenesulfonic acid, 4 methylbenzenesulfoc acid, naphthalene 2 sulfonic acid, naphthalene 1,5 disulfonic acid, 2 or 3 phosphoglycerate, glucose 6 phosphate, N cyclohexylsulfamic acid (with the formation of cyclamates), or with other acid organic compounds, such as ascorbic acid.

Specifically, BH4 salts with inorganic or organic acids are preferred. Nonlimiting examples of alternative BH4 salts forms includes BH4 salts of acetic acid, citric acid, oxalic acid, tartaric acid, fumaric acid, and mandelic acid.

The frequency of BH4 dosing will depend on the pharmacokinetic parameters of the agent and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435 1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels of Phe in conjunction with relevant dose response data. The final dosage regimen will be determined by the attending physician, considering factors which modify the action of drugs, e.g., the drug's specific activity, severity of the damage and the responsiveness of the patient, the age, condition, body weight, gender and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

In certain aspects of the present invention, all the necessary components for the treatment of the diseases described herein using BH4 either alone or in combination with another agent or intervention traditionally used for the treatment of such disease may be packaged into a kit. Specifically, the present invention provides a kit for use in the therapeutic intervention of the diseases described herein comprising a packaged set of medicaments that comprise BH4 or a derivative or precursor thereof as well as buffers and other components for preparing deliverable forms of said medicaments, and/or devices for delivering such medicaments, and/or any agents that are used in combination therapy with such BH4-based medicaments, and/or instructions for the treatment of the diseases described herein packaged with the medicaments. The instructions may be fixed in any tangible medium, such as printed paper, or a computer-readable magnetic or optical medium, or instructions to reference a remote computer data source such as a world wide web page accessible via the internet.

### III. Factors that Alter BH4 Synthesis and/or NO Production

The present invention contemplates a method of treating a disease or disorder characterized by endothelial dysfunction comprising administering to said subject a composition comprising an agent that increases tetrahydrobiopterin (BH4) or a precursor or derivative thereof alone or in combination with a therapeutic agent wherein said administration is effective in alleviating endothelial dysfunction of said subject as compared to said endothelial dysfunction in the absence of said BH4-containing composition.

One embodiment of the invention includes one or more agents that increase BH4 levels by increasing the expression or synthesis or the activity of the enzymes in the BH4 synthetic pathway including the first and the rate-controlling enzyme GTPCH1, PTPS and SR. In a preferred embodiment of the invention, BH4 synthesis is increased by increasing the expression of GTPCH1 expression by the use of any one or more cyclic adenosine monophosphate (cAMP) analogs or agonists including forskolin, 8-bromo cAMP or other agents that function to increase cAMP mediated cell signaling, for example, cytokines and growth factors including interleukin-1, interferon-gamma (IFN-gamma), tumor necrosis factor alpha (TNF-alpha), c-reactive protein, HMG-CoA-reductases (statins like atorvastatin) nerve growth factor (NGF), epidermal growth factor (EGF), hormones including adrenomedullin and estradiol benzoate, and other compounds such as NADPH and NADPH analogs, caffeine, cyclosporine A methyl-xanthines including 3-isobutyl-1-methyl xanthine, theophylline, reserpine, hydrogen peroxide.

It is well established in the art that the phosphodiesterases degrade the 3′5′-cyclic nucleotides such as cGMP and cAMP. cAMP is a known activator of GTPCH1 the rate controlling enzyme for BH4 synthesis the required co-factor for eNOS. Inhibitors of phosphodiesterases family, thus, have a secondary activating effect on BH4-synthetic enzyme GTPCH1. One embodiment of invention therefore relates to increasing GTPCH1 levels by inhibiting the degradation of 3′5′-cyclic nucleotides using inhibitors of the eleven phosphodiesterases families (PDE1-11) including PDE1, PDE3, PDE5. The PDE inhibitors of the present invention include Viagra/tadalafil, cialis/sildanefil, vardenafil/levitra, 8-Methoxymethyl-IBMX, UK-90234, dexamethasone, hesperetin, hesperedins, Irsogladine, vinpocetine, cilostamide, rolipram, ethyl beta-carboline-3-carboxylate (beta-CCE), tetrahydro-beta-carboline derivatives, 3-O-methylquercetin and the like.

Another embodiment of the invention relates to increasing the levels of BH4 by increasing the levels of BH4-synthesizing enzymes by gene therapy or endothelium-targeted delivery of polynucleotides of the synthetic machinery of BH4. Patents filed claiming BH4-synthesizing genes to be used in gene therapy include US20030198620. Yet another embodiment of the invention relates to increasing the levels of BH4 by supplementation with BH4-synthesizing enzymes GTPCH1, PTPS, SR, PCD, DHPR and DHFR. It is contemplated that by BH4-synthesizing enzymes, it is asserted that all natural and unnatural forms of the enzymes including mutant of the proteins active and inactive are included.

Another embodiment of the invention relates to increasing BH4 levels by diverting the substrate 7,8-dihydroneopterin triphosphate towards BH4 synthesizing enzyme PTPS instead of alkaline phosphatase (AP) by inhibiting AP activity. The agents or compounds that inhibit the activity of AP include phosphate analogs, levamisole, and L-Phe. Another embodiment of the invention relates to agents or compounds that inhibit alkaline phosphatase includes the small inhibitory RNA (siRNA), antisense RNA, dsDNA, small molecules, neutralizing antibodies, single chain, chimeric, humanized and antibody fragments to inhibit the synthesis of alkaline phosphatase.

Another embodiment of the invention includes agents or compounds that enhance the activity of catalysts or cofactors needed for the synthesis of enzymes of the de novo synthesis pathway of BH4 synthesis.

Another embodiment of the invention includes agents or compounds that prevent the degradation of the enzymes needed for the synthesis of BH4. Yet another embodiment of the invention includes agents or compounds that prevent the degradation of the catalysts needed for the synthesis of BH4 and its synthetic enzymes including GTPCH1, PTPS and SR.

Another embodiment of the invention relates to increasing BH4 levels by inhibiting the feedback modulation of the GTPCH1/GFRP complex by BH4. A preferred embodiment of the invention relates to agents or compounds that inhibit the binding of BH4 to the GTPCH1/GFRP complex, thereby preventing the feedback inhibition by BH4. Agents or compounds of this invention include competitive inhibitors such as alternate forms of BH4 with altered affinities for the complex, structural analogs etc. Still another embodiment of the invention includes agents or compounds that enhance the binding of L-phenylalanine to CTPCH1/GFRP inducing the synthesis of BH4. Another embodiment of the invention includes agents or compounds that increase the levels of L-Phe such as precursors of L-Phe.

Yet another embodiment of the invention relates to agents or compounds that modulate the activity or the synthesis of GFRP. A preferred embodiment of the invention includes agents or compounds that inhibit the activity of GFRP. Another embodiment of the invention includes the use of siRNA, small molecules, antibodies, antibody fragments and the like to inhibit the synthesis of GFRP.

Another embodiment of the invention relates to increasing the levels of BH4 by increasing the reduction of BH2 via the salvage pathway. In vivo, BH4 becomes oxidized to BH2. BH2 which exist as the quinoid form (qBH2) and as the 7,8-dihydropterin which is reduced to BH4 by DHPR and DHFR respectively. A preferred embodiment of the invention relates to increasing the regeneration or salvage of BH4 from BH2 by modulating the activity and synthesis of the enzymes PCD, DHPR and DHFR using agents or compounds that pathway NADPH, thiols, perchloromercuribenzoate, hydrogen peroxide and the like.

Another embodiment of the invention relates to increasing the levels of active BH4 by decreasing the oxidation of BH4 using agents or compounds such as antioxidants including ascorbic acid (vitamin C), vitamin E, tocopherols (e.g vitamin A), selenium, beta-carotenes, carotenoids, flavones, flavonoids, folates, flavones, flavanones, isoflavones, catechins, anthocyanidins, chalcones etc. US patents and patent applications U.S. Pat. No. 6,544,994, US20050119270, US20030007961, and US20020052374 describe the use of BH4 and antioxidant.

Yet another embodiment of the invention includes agents or compounds that are the precursors of BH4 including guanosine tripohosphate, 7,8-dihydro-neopterin triphosphate and 6-pyrovoyl tetrahydropbiopterin.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Clinical Evaluation With 6R-Tetrahydrobiopterin

The following example provides guidance on the parameters to be used for the clinical evaluation BH4 in the therapeutic methods of the present invention. As discussed herein throughout, BH4 will be used in the treatment of diabetes-related and non-diabetic cardiovascular complications, including but not limited to resistant hypertension, intermittent claudication, coronary hypertension, coronary artery function, pulmonary arterial hypertension, and hemolytic anemias including sickle cell disease. Clinical trials will be conducted which will provide an assessment of daily oral doses of BH4 for safety, pharmacokinetics, and initial response of both surrogate and defined clinical endpoints. The trial will be conducted for a minimum, but not necessarily limited to 1 week for each patient to assess efficacy in reversing the relevant study endpoints, e.g. development of pain during walking for intermittent claudication, and to collect sufficient safety information for 30 evaluable patients.

The initial dose for the trials will vary from about 2 to about 10 mg/kg, or from about 1 to 20 mg/kg. In the event that this dose does not produce an improvement in the clinical endpoint in a patient, or produce a significant direct clinical benefit, the dose should be increased as necessary, and maintained for an additional minimal period of, but necessarily limited to, 1 weeks to establish safety and to evaluate further efficacy. Lower doses, e.g., doses of between 0.1 to 2 mg/kg also are contemplated, as are doses of 1 mg/kg to 5 mg/kg. Such doses are expected to provide improvements with relevant study endpoints, including but not limited to those relating to atherosclerotic complications or pulmonary hypertension.

The invention specifically contemplates the use of BH4, or a precursor or derivative thereof, for treating any of the vascular disease states mentioned in the present application or any of the vascular disease states mentioned in U.S. application Ser. No. 11/143,887 filed Jun. 1, 2005, incorporated herein by reference in its entirety, at a dose in the range of 1 to 5 mg/kg body weight/day, via any route of administration including but not limited to oral administration, in a once daily dose or multiple (e.g. 2, 3 or 4) divided doses per day, for a duration of at least 1, 2, 3, or 4 weeks or longer, or 1, 2, 3, 4, 5, 6 months or longer. Exemplary doses include less than 5 mg/kg/day, 4.5 mg/kg/day or less, 4 mg/kg/day or less, 3.5 mg/kg/day or less, 3 mg/kg/day or less, 2.5 mg/kg/day or less, 2 mg/kg/day or less, 1.5 mg/kg/day or less, 1 mg/kg/day or less, or 0.5 mg/kg/day or less. Equivalent doses per body surface area are also contemplated.

For the person of average weight/body surface area (e.g. 70 kg), the invention also contemplates a total daily dose of less than 400 mg. Exemplary such total daily doses include 360 mg/day, 350 mg/day, 300 mg/day, 280 mg/day, 210 mg/day, 180 mg/day, 175 mg/day, 150 mg/day, or 140 mg/day. For example, 350 mg/day or 175 mg/day is easily administrable with an oral dosage formulation of 175 mg, once or twice a day. Other exemplary total daily doses include 320 mg/day or less, 160 mg/day or less, or 80 mg/day or less. Such doses are easily administrable with an oral dosage formulation of 80 or 160 mg. Other exemplary total daily doses include 45, 90, 135, 180, 225, 270, 315 or 360 mg/day or less, easily administrable with an oral dosage formulation of 45 or 90 mg. Yet other exemplary total daily doses include 60, 120, 180, 240, 300, or 360 mg/day, easily administrable with an oral dosage formulation of 60 or 120 mg. Other exemplary total daily doses include 70, 140, 210, 280, or 350 mg/day, easily administrable with an oral dosage formulation of 70 or 140 mg. Exemplary total daily doses also include 55, 110, 165, 220, 275 or 330 mg/day, easily administrable with an oral dosage formulation of 55 mg. Other exemplary total daily doses include 65, 130, 195, 260, or 325 mg/day, or 75, 150, 225, 300 or 375 mg/day, e.g. in dosage formulations of 65 mg or 75 mg.

If BH4 itself is being administered, any of the salts or polymorph forms described in U.S. application Ser. No. 11/143,887 and/or the stable solid formulation described in Int'l Application No. PCT/US05/41252 filed Nov. 16, 2005, incorporated herein by reference in its entirety, may be administered. Exemplary precursors and derivatives of BH4 that retain its beneficial activity are described in U.S. application Ser. No. 11/143,887.

Measurements of safety will include adverse events, allergic reactions, complete clinical chemistry panel (kidney and liver function), urinalysis, and CBC with differential. In addition, other parameters also will be monitored. The present example also contemplates the determination of pharmacokinetic parameters of the drug in the circulation, and general distribution and half-life of 6R-BH4 in blood. It is anticipated that these measures will help relate dose to clinical response.

Methods

Patients who have diabetes-related and non-diabetes related vascular disorders will undergo a baseline a medical history and physical exam, and various diagnostic tests commonly used to diagnose the specific indication (e.g. development of pain during treadmill test in patients with intermittent claudication) in the clinical setting including but not limited to measurement of blood pressure, six-minute walk test and echocardiography studies. The proposed human dose of 2 to about 10 mg/kg BH4 will be administered divided in one to three daily doses. Clinical endpoints will be monitored at frequent intervals. A complete evaluation will be conducted one week after completing the treatment period. Should dose escalation be required, the patients will follow the same schedule outlined above. Safety will be monitored throughout the trial.

Diagnosis and Inclusion/Exclusion Criteria

Subjects will be selected based on gender, age and documented diagnosis of the specific vascular disorder confirmed by common diagnostic tests.

Dose, Route and Regimen

Patients will receive BH4 at a dose of 5 mg/kg per day. In the event that the clinical endpoint is not improved by a reasonable amount and no clinical benefit is observed, the dose may be increased as necessary until a total daily dose of 20 mg/kg is administered. The daily BH4 dosage will be administered orally or via nasogastric tube as liquid, powder, tablets or capsules. The total daily dose may be given as a single dose or perhaps divided in two or three daily doses. The patients will be monitored clinically as well as for any adverse reactions. If any unusual symptoms are observed, study drug administration will be stopped immediately, and a decision will be made about study continuation.

BH4 Safety

BH4 therapy will be determined to be safe if no significant acute or chronic drug reactions occur during the course of the study. The longer-term administration of the drug will be determined to be safe if no significant abnormalities are observed in the clinical examinations clinical labs, or other appropriate studies.

### EXAMPLE 2

### Clinical Evaluation With 6R-Tetrahydrobiopterin in Diabetics Establishment of Dose Effect, Dose Interval and Safety in Diabetics in Phase 1/2 or 2a

Prior to initiating any phase 2a dosing/efficacy studies, a short phase 1/2 dose escalation study will be conducted in a variety of diabetic populations to establish the dose effect on vascular compliance and safety should be considered. The first study will establish dose range and regimen and the range of vascular function endpoints that can be monitored to support clinical endpoints. Subjects of the first study will be diabetics with significant vascular disease (reduced microvascular compliance) and hypertension, and daily oral doses of BH4 from 0, 1, 2, 5, 10 and 20 mg/kg will be administered in a once daily or twice daily dosing regimen. The patients will be monitored for vascular compliance, perfusion/reperfusion/forearm blood flow and blood pressure over a week of treatment and within the span of a day once stabilized on a dose. The study will evaluate the appropriate dose range and the daily regimen in phase 2 and establish the presence of quantitative efficacy measures to be used to support the clinical development, as well as safety in this population.

### Exploration of Indications in the Diabetic Population in Phase 2a and 2b

The appropriate population, dose/regimen and indications will be evaluated in the Phase 2a program in two or more studies with relatively shorter in life treatment periods of 30-60 days. In any narrowly focused group of diabetic patients with particular medical need that is included in any Phase 2a study, a variety of other measures will be evaluated in parallel to assess other clinical impacts of changes in vascular dysfunction as well as evaluations of biochemical markers of endothelial function and insulin resistance.

A Phase 2b study in the appropriate population/indication will be performed to test out a longer treatment period, to assist with the design of a Phase 3 study in a larger population, and evaluate specific doses.

### Phase 3 Studies of the Chosen First Indication in the Selected Diabetic

Phase 3 studies will be focused on the key indication but accrual of supportive information on vascular function, as secondary or tertiary endpoints will be included. A minimum of two controlled Phase 3 studies will be conducted but additional Phase 2 or Phase 3 designs will be considered to include other populations at risk such as patients with different concomitant medications, patients with particular medical problems such as renal failure, or patients at other ends of the spectrum of disease.

### General Vascular Function

Vascular compliance/endothelial dysfunction/HTN: A variety of diabetics with evidence of changes in vascular compliance and C2 abnormalities and HTN will be assessed for C2 measure of vascular compliance in smaller vessels measured during one week of escalating doses from 0, 1, 2, 5, 10 and 20 mg/kg. The effects of increasing BH4 on the vascular compliance and HTN will be assessed and the dose effect of BH4 will be established in a varied population of patients. The drug effect over the course of a day will be determined to help establish the regimen

Recalcitrant hypertension: A Phase 2a study will be initiated and based on a randomized, double blind placebo study design. The study will involve 80 patients with blood pressures greater than 140 mmHg/90 mmHg and using three medications. The total number of patients will include 40 diabetic patients. There will be 20 patients per group and four groups including the placebo group. Exclusion criteria will include critical limb ischemia, heart failure and orthopedic complications. BH4 will be administered to patients in a dosage range of 0 to 10 mg/kg for four weeks. Patients will be evaluated for measurements of systolic and diastolic blood pressure, and monitored for ambulatory blood pressure.

Insulin sensitivity/glucose control: Diabetics with poor glucose control and unresponsive or poorly responsive to common antidiabetic oral agents will be assessed based on measurement of glucose/insulin levels, Hgb A1c. The administration of BH4 is expected to improve results in the glucose tolerance test and glucose control (decreased Hgb A1C). Such patients may be assessed in various protocols

Peripheral Perfusion

Studies in Specific Diabetes-Related Indications

Intermittent claudication: A Phase 2 a study will be conducted based on a 1:1, double-blind placebo-controlled study design. The study will include 80 patients with intermittent claudication, including 40 diabetic patients. Exclusion criteria will include critical limb ischemia, heart failure and orthopedic complications. Patients will be given either a placebo or BH4 in a dosage of 10 mg/kg for 12 weeks. Patients with calf pain and limitations in ability to walk longer distances will be evaluated for their ability to walk over a period of time and distance on a treadmill until pain begins. Specifically the clinical relevant endpoints of peak walking time by graded treadmill, pain-free walking distance, blood pressure, blood flow, flow mediated dilation (FMD) and quality of life (QoL). The administration of BH4 is expected to improve the vascular dilatory response to exercise and ability to walk over longer distances, which is the most clinically relevant endpoint for peripheral perfusion.

Poor peripheral perfusion: Patients with poor blood flow to extremities will be evaluated with respect to improvement in blood flow to extremities with escalating BH4 doses and reduced frequency of amputation.

Poor skin blood flow and wound healing: Patients with diabetic ulcers or poor skin blood flow will be evaluated with respect to improvements in blood flow to skin, and enhanced healing of wounds with BH4 therapy.

Cardiac Disease

Congestive heart failure (CHF) or Pulmonary Hypertension: Diabetics with CHF complicated by increased blood pressure and poor vascular function will be evaluated based on measurements provided by echocardiography, cardiac output/ejection fraction, and six-minute walk test. BH4 therapy will be expected to decrease blood pressure, enhance cardiac blood flow to synergistically improve cardiac function, and improve six-minute walk test.

Angina during exercise: Diabetics with exercise-limiting angina requiring nitrate therapy will be assessed by measuring time that subject is able to walk on a treadmill until the development of angina while on BH4 and by monitoring coronary blood flow. BH4 therapy is expected to improve coronary flow and thereby delay or eliminate angina as tested on the treadmill.

Coronary artery disease (CAD) and related atherosclerosis: Diabetics with early or late-stage atherosclerosis and coronary artery disease, with one prior myocardial infarction (MI) or an ischemic event will be evaluated with respect to prevention of progression of atherosclerosis and/or reduction in MI/stroke/death. A Phase 2 a study will be conducted based on a randomized, double blind, placebo-controlled study design. The study will involve 80 patients including 40 diabetics presenting with CAD requiring coronary artery bypass grafting (CABG). There will 20 patients per group and four groups including a placebo group. Exclusion criteria will be the occurrence of heart failure and unstable angina. Patients will be given 0-10 mg/kg BH4 or a placebo for four weeks. Patients will be evaluated for coronary artery dysfunction, large vessel dysfunction, CAD, tissue levels of reactive oxygen species, and BH4 levels. BH4 therapy is expected to decrease the rate of progression of atherosclerosis, frequency of MI and other vascular events. Acutely, the improved vasodilation and reduce thrombogenicity will be expected to enhance coronary function, and later reduced atherosclerosis will be expected to improve progression in vessel disease.

Opthalmologic Disease

Optic atrophy or diabetic retinal disease: Diabetics with declining visual acuity due to retinal vascular insufficiency will be evaluated with respect to visual acuity and blood flow measurement to retina. BH4 is expected to improve blood flow leading to improved vision.

Renal Disease

Microalbuminuria in diabetic renal disease or renal failure/reduced glomerular filtration rate (GFR): Diabetics with protein loss in urine consistent with vascular disease of kidney or elevated creatinine levels will be assessed with respect to measurement of protein loss in urine in 24 hour specimens and GFR. BH4 therapy is expected to reduce proteinuria and improve GFR.

Pulmonary Hypertension in Diabetics

Diabetics with pulmonary hypertension with or without congestive heart failure will be evaluated with respect to measurement of ability to perform six-minute walk test, development of CHF, and measurement of pulmonary pressure. BH4 therapy is expected to improve pulmonary pressure and cardiovascular performance. Some data exists in animal models that BH4 deficiency and pulmonary hypertension are related.

### EXAMPLE 3

### Studies in Other Cardiovascular Indications Unrelated to Diabetes Pulmonary Vascular Disease

A Phase 1, Multicenter, Open-Label, Dose-Escalation Study to Evaluate the Safety and Efficacy of 6R-BH4 in Subjects with Pulmonary Arterial Hypertension

### Objectives:

The primary objective of the study is to evaluate the safety of oral 6R-BH4, administered in escalating doses in addition to standard care, in subjects with pulmonary arterial hypertension (PAH).

The secondary objective of the study is to evaluate change in biochemical markers of endothelial dysfunction and nitric oxide synthetase activity (coupled and uncoupled) in subjects with PAH receiving escalating doses of oral 6R-BH4 in addition to standard care.

The tertiary objective of the study is to evaluate change in biomarkers of disease progression, 6-minute walk (6MW) distance, Borg dyspnea scores, and quality of life (QOL) measures in subjects with PAH receiving escalating doses of oral 6R-BH4 in addition to standard care.

### Methodology:

This is a Phase 1, multicenter, open-label, dose-escalation study in subjects with PAH whose symptoms have been clinically stable for 8 weeks prior to enrollment. Eligible subjects will undergo baseline assessments before beginning daily oral treatment with 6R-BH4. During the treatment period, the planned 6R-BH4 dose-escalation regimen for all subjects will be 2.5 mg/kg/day for two weeks, 5 mg/kg/day for two weeks, 10 mg/kg/day for four weeks, then 20 mg/kg/day for two days. The first 5 subjects enrolled for the first dose level will be admitted to the study unit for 24 hours for safety assessment. If the 24-hour safety profile for these five subjects is considered acceptable by the principal investigators, then for each subject enrolled thereafter, the first dose of each dose level (except 20 mg/kg/day) will be administered in the study unit and the subject will be monitored for 4 hours before leaving. At the 20 mg/kg/day dose level, each subject will be admitted to the study unit for 48 hours for dose administration. For each subject, an acceptable safety profile at each dose level will be required prior to dose escalation. In addition, the safety profile of the planned dose-escalation regimen will be evaluated throughout the study. Subjects will return after a four-week washout period for follow-up assessments. Enrollment will continue until 20 subjects have completed the washout period and follow-up assessments.

The key criteria for including subjects are as follows: documented diagnosis of PAH, defined as mean pulmonary arterial pressure >25 mm Hg (measured by catheter); PAH is primary (idiopathic) or is secondary and caused by collagen vascular disease, congenital heart disease, or thromboembolic disease; Modified New York Heart Association (NYHA) (WHO) classification I, II, or III that has been stable for at least 8 weeks prior to enrollment; 6-minute walk distance, as performed at screening or within three months (12 weeks) prior to screening, of ≧200 and ≦500 meters; receiving stable doses of one or more medications that are approved for treatment of PAH, except for any agents specifically prohibited by this protocol, for a minimum of 8 consecutive weeks before enrollment.

### Dose of BH4 and Mode of Administration:

6R-BH4 at 2.5, 5.0, 10, or 20 mg/kg will be administered once daily, dissolved in water and taken within one hour after the morning meal. The duration of treatment will be approximately 8.5 weeks (54 to 62 days).

### Safety

Safety will be assessed by performing physical examinations; serially measuring BP, heart rate, and oxygen saturation; serially assessing signs and symptoms of PAH; recording adverse events and concomitant medications; monitoring laboratory parameters (chemistry, hematology, coagulation panel, thyroid function and urinalysis); and serially assessing electrocardiograms, echocardiograms (two dimensional and/or Doppler), and NYHA (WHO) classification.

### Efficacy

Efficacy will be assessed by change in biochemical markers (‘biomarkers’) of endothelial dysfunction, nitric oxide synthetase activity (coupled and uncoupled), and disease progression: exhaled nitric oxide (NO), at rest; urinary isoprostanes; urinary cGMP; plasma B-type natriuretic peptide (BNP); C-reactive protein (CRP); monocyte chemoattractant protein-1 (MCP-1).

Efficacy will also be assessed by change in 6-minute walk (6MW) distance, Borg dyspnea score (after 6MW), and results from the Cambridge Pulmonary Hypertension Outcome Review (CAMPHOR) QOL questionnaire.

### Other Studies of Pulmonary Vascular Disease

Pulmonary hypertension in sickle cell or other hemoglobinopathies (Hb): Hb-S patients with pulmonary hypertension with or without congestive heart failure will be evaluated with respect to ability to perform the six-minute walk test, development of CHF, and measurement of pulmonary pressure. BH4 therapy is expected to improve pulmonary pressure and cardiovascular performance

Idiopathic pulmonary hypertension (IPH): IPH patients with no known cardiovascular cause for their disease, and particularly young patients under the age of 40 years will be evaluated with respect to the six-minute walk test, development of CHF, and measurement of pulmonary pressure. BH4 therapy is expected to improve pulmonary pressure and CV performance

Persistent pulmonary hypertension of the newborn (PPHN): Newborns from term pregnancy who get PPHN will be evaluated with respect to resolution of pressures, restoration of oxygenation, and mortality rate. BH4 therapy is expected to cause rapid and profound reversal of pulmonary artery pressures and shunting resulting in improved oxygenation. Unlike nitric oxide inhalation therapy, which is impractical and toxic, BH4 would be nontoxic and effective.

### Stroke and Related Ischemic Vascular Disease

Post-stroke cerebrovascular spasm: Subjects will include post-stroke patients hospitalized and treated for acute stroke. They will be evaluated with respect to infarct size and pre- and post-treatment brain perfusion. BH4 therapy is expected to cause relaxation of the spasm and reduce the size of the infarct. Data from a canine stroke model shows that post-stroke vasospasm around the site of the clot, causes extension and greater damage than the original event and can be prevented by infusing nitrite solutions.

### Transplant-Related Endothelial Dysfunction

Vascular dysfunction after solid organ transplantation: The study will include patients who have undergone solid organ transplant. The subject will be evaluated for organ dysfunction caused by vascular dysfunction following transplant. BH4 is expected to improve organ function post transplant and reduce loss of the transplanted organ.

Cyclosporin A (CsA) induced endothelial dysfunction: Patients on CsA after organ transplant will be evaluated with respect to organ dysfunction caused by vascular dysfunction following transplantation. BH4 therapy is expected to improve organ function and reduce the frequency of vascular complications.

Brandacher et al., Transplantation, 81(4):583 (2006) reports that allograft survival was significantly prolonged by tetrahydrobiopterin and cyclosporine A. Compared to allogeneic untreated controls, intragraft peroxynitrite formation was lowered in all groups. Briefly, two fully allogeneic strains of inbred, male mice were obtained. Cervical heterotopic heart transplantation was performed and cardiac allograft survival was determined by daily palpation and inspection, with complete cessation of heart beats indicating severe rejection, which was confirmed by histology. Groups of 5-10 animals were treated for 7 days following transplantation with BH4 (50 mg/kg every 8 hours, freshly dissolved in phosphate buffered saline and administered intramuscularly). Mice were sacrificed on postoperative days 1 or 6 and the transplants removed for histological evaluation. Corresponding plasma samples were harvested for nitrate and nitrite measurements by HPLC. Tissue samples were either immediately frozen in liquid nitrogen and stored at −75 C, or fixed in formalin and embedded in paraffin until analyzed by H&E staining or immunohistochemistry. Untreated allografts were rejected around days 7 and 8 posttransplantation (mean graft survival time 7.1±0.7 days) whereas tetrahydrobiopterin prolonged survival to 12.3±4.9 days (p<0.05 different from untreated allografts). On histological evaluation, BH4-treated hearts showed infiltrates of mononuclear cells and some foci of inflammatory infiltrate. Nitrate and nitrite concentrations were elevated on day 6 in untreated animals and in animals treated with BH4. The data showed that tetrahydrobiopterin significantly prolonged allograft survival.

### Cardiac or Coronary Disease

A Randomized, Placebo-Controlled Study of Two Doses of Oral 6R-BH4 on Vascular Function in Subjects with Coronary Artery Disease

Objectives

The primary objective is to compare vascular function in subjects with coronary artery disease before and after treatment with different doses (400 mg or 700 mg daily) of 6R-BH4, as assessed by non-invasive MRI. The principal markers of vascular function will be aortic distensibility and pressure pulse wave velocity, carotid artery distensibility and brachial artery flow-mediated dilatation.

The secondary objectives are: (1) to assess the effect of 6R-BH4 therapy on the following markers of vascular function in isolated blood vessels: vascular and myocardial superoxide production and nitric oxide-mediated endothelial function by organ bath isometric tension studies; and (2) to assess the effect of 6R-BH4 therapy on biopterin levels in isolated blood vessels.

The tertiary objectives are: (1) to assess the effect of 6R-BH4 therapy on insulin resistance (fasting glucose & insulin levels) in type II diabetics; (2) to assess the effect of 6R-BH4 therapy on systemic blood pressure; (3) to assess the effect of 6R-BH4 therapy on microalbuminuria; (4) to assess the effect of 6R-BH4 therapy on urinary isoprostane levels; (5) to assess the effect of 6R-BH4 therapy on: incidence of post-operative AF; length of ITU stay; length of hospital stay; and renal function; and (6) to assess the effect of 6R-BH4 therapy on blood markers of oxidative stress and endothelial function.

The safety objective is to assess the safety of 400 or 700 mg/day of oral dosing of 6R-BH4 in subjects with CAD.

Endpoints

The primary endpoint will be the effects of 6R-BH4 (400 mg or 700 mg daily) on the following MRI parameters of vascular function in subjects with CAD: brachial artery reactivity, aortic distensibility, aortic pressure pulse wave velocity, and carotid artery distensibility.

The secondary endpoints include: (1) effect of 6R-BH4 therapy on nitric oxide-mediated endothelial function by organ bath isometric tension studies; (2) effect of 6R-BH4 therapy on vascular and myocardial superoxide production; and (3) effect of 6R-BH4 therapy on tissue biopterin levels in isolated blood vessels.

The tertiary endpoints include: (1) effect of BH4 therapy on insulin resistance (fasting glucose & insulin levels) in type II diabetics; (2) effect of 6R-BH4 therapy on systemic blood pressure; (3.) effect of 6R-BH4 therapy on urinary microalbuminuria as measured by albumin to creatinine ratio; (4) effect of 6R-BH4 therapy on urinary isoprostane levels; (5) effect of 6R-BH4 therapy on: incidence of post-operative AF as assessed by R-test recording and ECG; length of hospital stay; and serum creatinine concentration as a marker of renal function

The safety endpoint will be to assess the overall safety by evaluation of:

ECG, clinical laboratory tests (CBC, Chemistry), adverse events, and concomitant medications.

Study Design and Treatment

The trial will be a placebo-controlled, double blind trial.

Subjects will be randomized to one of 3 treatment groups following MRI scanning at this first visit. See FIG. 30. The study treatments are as follows: (1) Oral 6R-BH4 400 mg daily dissolved in water (at night); (2) Oral 6R-BH4 700 mg daily dissolved in water (at night); (3) placebo daily dissolved in water (at night). Each subject will take 7 tablets PO, dissolved in water, each night. The 7 tablets will comprise either 7 placebo tablets (placebo group), 4×100 mg 6R-BH4 tablets and 3 placebo tablets (400 mg 6R-BH4 group), or 7×100 mg 6R-BH4 tablets (700 mg 6R-BH4 group). Subjects will take their dose within 1 hour after the last (evening) meal of the day. Dosing instructions would be the following: The tablets will be dissolved in at least 4 oz (120 mL) of water. Subjects will be instructed to ingest all of the solution within 15 minutes of dissolving the tablets. To ensure that the full dose is taken, any undissolved study drug remaining in the glass should be rinsed with 1-2 oz, or 30-60 mL of water and ingested immediately.

A key criteria for including a subject includes multivessel coronary artery disease scheduled to undergo coronary bypass surgery.

On the first visit, the following information will be obtained: informed consent, demographics, medical history, physical examination, MRI, and prescription of medication. Subjects will be instructed on the correct method of dosing. The second visit will take place about 4 weeks later and prior to CABG and information with respect to update of adverse effects, concomitant medications, measurement of blood pressure and heart rate, second MRI scan, and blood and urine sampling. Visit 3 will be the day of the CABG. During the operation, samples of the artery (internal mammary or radial) vein (saphenous) and myocardial tissue will be obtained. During in-hospital recovery, the patient will continue to receive the study medication, evaluated by ECG daily for the presence of atrial fibrillation, and blood samples will be collected on the second postoperative day to assess plasma levels of biomarkers of the vascular disease.

Vascular dysfunction/angina: Hypercholesterolemic patients and smokers will be evaluated with respect to prevention of progression of atherosclerosis and/or reduction in MI, stroke or death. BH4 therapy is expected to decrease the rate of progression of atherosclerosis, improve coronary vasodilation and decrease thrombogenicity.

Congestive heart failure: Non-diabetic patients with CHF will be assessed with respect to development of CHF using echocardiography, cardiac output/ejection fraction, and six-minute walk test. BH4 therapy is expected to decrease blood pressure, improve cardiac blood flow to synergistically enhance cardiac function, and improve 6-minute walk test.

Hemolytic Anemias—Sickle Cell Disease

Hemolytic anemia is characterized by an inadequate number of circulating red blood cells (anemia) resulting from the premature destruction of red blood cells. Some of the causes of hemolytic anemia include infection, drug therapy, autoimmune disorders and genetic disorders. There are various types of hemolytic anemias including but not limited to sickle cell anemia, paroxysmal nocturnal hemoglobinuria, hemoglobin SC disease, hereditary elliptocytosis, hereditary ovalocytosis, idiopathic autoimmune hemolytic anemia, non-immune hemolytic anemia caused by chemical or physical agents, secondary immune hemolytic anemia, and thalassemia. Some common treatments for hemolytic anemia include folic acid, iron replacement, and corticosteroids, and transfusion of blood in emergencies. The type of treatment, prognosis and complications may vary with the type of hemolytic anemia. Complications include cardiovascular collapse and aggravation of pre-existing heart disease, lung disease, or cerebrovascular disease.

Patients will be evaluated for symptoms including chills, fatigue, pale skin color, shortness of breath, rapid heart rate, jaundice, dark urine, and enlarged spleen The presence of hemolysis will be determined by detection of elevated indirect bilirubin levels, low serum haptoglobin, hemoglobin in the urine, hemosiderin in the urine, increased urine and fecal urobilinogen, elevated absolute reticulocyte count, low red blood cell count (RBC) and hemoglobin, and elevated serum LDH. The direct measurement of the red cell life span by isotopic tagging techniques will be used to measure life span. Once hemolysis has been established, more specific tests will be used to identify the specific types of hemolytic anemia. Other relevant measures that are affected by the disease will include uric acid, TIBC, RBC indices, protein electrophoresis—serum, potassium test, platelet count, peripheral smear, leukocyte alkaline phosphatase, serum iron, hematocrit, ferritin, febrile or cold agglutinins, Donath-Lansteiner test, direct and indirect Coombs' test, CBC, blood differential, AST, and 24-hour urine protein.

Sickle Cell Disease

Sickle cell disease also known as sickle cell anemia or hemoglobin SS disease (Hb SS) is an inherited disorder characterized by abnormal crescent shaped red blood cells that function abnormally causing small blood clots, which contribute to the development of recurrent painful episodes called “sickle cell pain crises.” Sickle cell disease is caused by an abnormal type of hemoglobin called hemoglobin S that polymerizes and distorts the shape of the red blood cell, and leads to premature destruction and/or rupture of the red blood cell. The fragile, sickle-shaped cells deliver less oxygen to the body's tissues, and can break into pieces that disrupt blood flow. Sickle cell anemia is inherited as an autosomal recessive trait and affects approximately one out of every 500 African Americans, as well as a number of other ethnicities. Although sickle cell disease is present at birth, symptoms commonly develop after 4 months of age and can become life threatening. Blocked blood vessels and damaged organs can cause acute painful episodes, or “crises”, including hemolytic crisis (breakdown of damaged red blood cells), splenic sequestration crisis (enlargement of the spleen due to accumulation of blood cells), and aplastic crisis (infection-induced cessation of bone marrow and red blood cell production). These painful crises can affect the bones of the back, the long bones, and the chest and may be severe enough to require hospitalization for pain control and intravenous fluids. Repeated crises can cause damage to the kidneys, lungs, bones, eyes, and central nervous system.

Treatment is chronic and includes supplementation with folic acid, an essential element in producing cells, is required because of the rapid red blood cell turnover. Therapy is focused on management and the control of symptoms and to try to limit the frequency of crises. Painful episodes are treated with analgesics and adequate liquid intake. Hydroxyurea (Hydrea) was found to help some patients by reducing the frequency of painful crises and episodes of acute chest syndrome and decreasing the need for blood transfusions. There is some concern but it has not been established that hydroxyurea may cause leukemia. Other newer therapies have been explored including agents that induce the body to produce more fetal hemoglobin to reduce the amount of sickling) or agents that increase the binding of oxygen to sickle cells. Bone marrow transplant can be curative, but is indicated in only a minority of patients due to the toxicity of drugs used in the management of transplantation, difficulty in finding suitable donors, and high expense. Antimicrobial agents and vaccines may be used to prevent bacterial infections common in children with sickle cell disease. Additional treatments may include partial exchange transfusion for acute chest syndrome; transfusions or surgery for neurological events, such as strokes, dialysis or kidney transplant for kidney disease, irrigation or surgery for priapism, surgery for eye problems, hip replacement for avascular necrosis of the hip, gallbladder removal in presence of significant gallstone disease, wound care, zinc oxide, or surgery for leg ulcers, drug rehabilitation and counseling for the psychosocial complications.

In the past, patients with sickle cell disease frequently died from organ failure between the ages of 20 and 40 years in most sickle-cell patients, but with improved management, survival has improved with patient living to the ages between 40 and 50 years. Causes of death include organ failure, infection and pulmonary arterial hypertension. Complications include recurrent aplastic and hemolytic crises resulting in anemia and gallstones, multisystem disease (kidney, liver, lung), narcotic abuse, splenic sequestration syndrome, acute chest syndrome, erectile dysfunction as a result of priapism, blindness/visual impairment, neurologic symptoms and stroke, joint destruction, gallstones, infection, including pneumonia, cholecystitis, osteomyelitis, and urinary tract infection, parvovirus B19 infection resulting in aplastic crisis, tissue death of the kidney, loss of function of the spleen, and leg ulcers.

Sickle cell anemia results from 2 carriers with sickle cell trait, and genetic counseling is recommended for all carriers of sickle cell trait (about 1 in 12 African Americans has sickle cell trait). Prenatal diagnosis of sickle cell anemia is available. Prompt treatment of infections, adequate oxygenation, and preventing dehydration may prevent sickling of red blood cells. Antibiotics and vaccinations may prevent infections. To prevent tissue deoxygenation, patient are advised to avoid strenuous physical activity, especially if the spleen is enlarged, emotional stress, environments with low oxygen content such as high altitudes and non-pressurized airplane flights, known sources of infection and dehydration.

A Phase 2a, Multicenter, Open-Label, Dose-Escalation Study to Evaluate the Safety, Tolerability, and Efficacy of 6R-BH4 in Subjects with Sickle Cell Disease

### Objectives

The primary objective is to evaluate the safety of oral 6R-BH4 administered in escalating doses to subjects with sickle cell disease (SCD). The secondary objective is to evaluate changes in physiological and biochemical markers of endothelial function in subjects with SCD receiving escalating doses of oral 6R-BH4. The tertiary objectives are to evaluate changes in clinical measures of SCD in subjects receiving oral 6R-BH4 for up to 2 years and to evaluate changes in the 6-minute walk (6MW) test in subjects receiving oral 6R-BH4 for up to 2 years.

### Methodology

This Phase 2a multicenter, open-label, dose-escalation study is designed to assess the safety and biologic activity of once daily (for 2.5, 5, and 10 mg/kg/day doses) or twice daily (for the 20 mg/kg/day dose) oral administration of 4 escalating doses of 6R-BH4 to subjects with SCD. During an optional extension phase, the study will assess the safety, tolerability, and efficacy of extended treatment with 6R-BH4.

Subjects will receive oral, once-daily (for 2.5, 5, and 10 mg/kg/day) or twice-daily (for 20 mg/kg/day) doses of 6R-BH4 during a 16-week dose-escalation phase, with dose levels increasing within subjects every 4 weeks, as follows: 2.5, 5, 10, and 20 mg/kg/day. Prior to dose escalation, the each subject will be evaluated with respect to safety profile during the previous dose level, using predefined parameters. If a subject does not meet the criteria for escalation to the next dose level or is de-escalated in dose, they will receive their highest tolerated dose for the duration of the 16-week period. Subjects who complete the 16-week dose-escalation phase and show improvement in physiological or biochemical markers of endothelial function, or who are deemed to derive clinical benefit from the study drug, will have the option to continue drug treatment for up to a total of 2 years. Subjects who continue into the extension phase will be treated at the highest dose level found to be both well tolerated and active in improving endothelial function during the dose-escalation phase.

During the dose escalation phase of the study, various assessments will be performed. Safety monitoring will include physical examination, vital signs, pulse oximetry, electrocardiogram (ECG), adverse event (AE) assessment, and changes in concomitant medications. Blood and urine samples will be collected and analyzed for routine clinical laboratory tests, brain-type natriuretic peptide (BNP), ferritin, markers of hemolysis (LDH, cell-free hemoglobin, methemoglobin, reticulocyte count), biochemical markers of endothelial function (plasma cyclic guanosine 3′,5′-monophosphate [cGMP], urinary 8-isoprostane, urine microalbumin, soluble vascular cell adhesion molecule-1 [sVCAM-1], soluble intercellular adhesion molecule-1 [sICAM-1], p-selectin, e-selectin and quantitative amino acids) and multi-analyte profile (MAP) testing for protein biomarkers. Endothelial function will be evaluated using Doppler echocardiogram (DE), peripheral arterial tonometry (EndoPAT), and exhaled nitric oxide (NO). The symptoms of SCD (frequency of sickle cell crises and vaso-occlusive crises), along with changes in the 6-minute-walk (MW) test, will be monitored.

Subjects who continue into the extension phase will be evaluated at 8-week intervals for a total of up to 2 years.

All subjects will have a safety follow-up phone contact at 2 weeks post treatment.

Up to 40 subjects will be screened and enrolled to ensure that subjects with a range of endothelial function abnormalities complete the 16-week dose-escalation phase of the study. Subjects who withdraw for reasons other than adverse events (AEs) may be replaced.

A key criteria for including a subject is the diagnosis of SCD, as confirmed by hemoglobin electrophoresis.

Subjects will receive 6R-BH4 provided as tablets that each contains 100 mg of sapropterin dihydrochloride. The number of tablets will be determined by the dose of sapropterin dihydrochloride, rounded to the nearest 100 mg. Tablets will be taken orally. Subjects taking all except the highest study drug dose (20 mg/kg/day) will take each dose in the morning within 1 hour after the morning meal. At the highest dose level (20 mg/kg/day), the total dose of 6R-BH4 will be divided-half of the tablets taken in the morning within 1 hour after a meal, and half approximately 12 hours later (or BID) within 1 hour after a meal. Subjects may decide whether to ingest study drug as whole tablets, or by dissolving the tablets in water, apple juice, or orange juice.

The study drug dose-escalation phase will be 16 weeks. The study has an optional extension phase, with a total treatment duration of up to 2 years (including the initial dose-escalation phase).

### Criteria for Evaluation

Safety criteria will be evaluated by physical examination, vital signs, pulse oximetry for O₂ saturation, ECG, clinical laboratory tests (complete blood count [CBC], chemistry, urinalysis), BNP, Ferritin levels, changes in markers of hemolysis such as lactate dehydrogenase (LDH), cell-free hemoglobin, methemoglobin, reticulocyte count, AE assessment, change in concomitant medications,

Efficacy criteria will be assessed based on endothelial function.

Changes in physiological assessments will be evaluated based on tricuspid regurgitant velocity (TRV) color-flow DE, as a surrogate for pulmonary arterial blood pressure, EndoPAT determination of endothelial function, and exhaled NO.

Changes in biochemical markers will be assessed by measurement of cGMP and urinary 8-isoprostane, urine microalbumin, sVCAM-1, sICAM-1, p-selectin, and e-selectin, and quantitative amino acids.

Clinical measures of SCD will be determined based on frequency of painful sickle cell crises (a crisis is defined as a visit to a medical facility for acute sickling-related pain that lasts more than 4 hours and is treated with a parenteral narcotic); frequency of vaso-occlusive crises, defined as any of the following: chest syndrome (chest wall pain, new pulmonary infiltrate on chest radiograph, fever), priapism, hepatic sequestration (sudden increase in liver size, pain in right upper quadrant, decrease in hemoglobin ≧2 g/dL, and increase in abnormal liver function tests not due to biliary disease); and change in 6MW test, as a clinical assessment of pulmonary arterial hypertension.

Additional Assessments include multi-analyte profile (MAP) testing of protein biomarkers,

Preliminary findings showed that patients treated with BH4 showed improvement in endothelial function as measured by the EndoPAT device available from Itamar Medical Ltd., of Caesarea, Israel.

### EXAMPLE 4

### BH4 Dosing for Hypertension in Clinical Studies

### Preliminary Studies

Prior studies have used parenteral infusion of BH4 or a single oral dose; thus it was not known whether long-term oral BH4 over a period of days to weeks would result in a sustained improvement in endothelial dysfunction. This study evaluate whether chronic oral therapy with BH4 in hypertensive patients resulted in reduction of arterial blood pressure (BP) as a result of improvement in endothelial dysfunction. Two studies were conducted to investigate the duration of action of oral BH4 and the response of hypertensive subjects to different doses of BH4.

Subjects between the ages of 18 and 75 years were recruited if they had uncontrolled hypertension on traditional stable anti-hypertensive therapy (BP≧135/85), or newly diagnosed hypertension (BP≧140/90). Patients were continued on their current anti-hypertensive therapy which remained unchanged. The criteria for exclusion were: female subjects with childbearing potential, history of recently symptomatic coronary or peripheral vascular disease, known secondary causes for hypertension, severe uncontrolled hypertension (BP>180 mmHg systolic and/or 110 mmHg diastolic), severe co-morbid conditions which would limit life expectancy to less than 6 months, renal or hepatic dysfunction, alteration of any concomitant anti-hypertensive therapy within the last 6 weeks, and any bleeding disorders. BH4 powder was obtained from Schircks Laboratories (Jona, Switzerland). BH4 when administered with vitamin C was compounded with vitamin C at a ratio of 1 mg of vitamin C/1 mg BH4 into appropriate sized capsules. Subjects were advised to keep the pills in a freezer or refrigerator.

Study 1: time of onset and duration of action of oral BH4:

Eight subjects were recruited, 3 male, mean age 60.5±10.9 years. Subjects were assigned to one of two groups. One group received BH4 10 mg/kg/day (n=4) and the other group received 5 mg/kg/day (n=4) given in two divided doses orally for 8 weeks. These dosages were based on dosing of BH4 used in phenylketonuria. Weekly BP measurements were made throughout the treatment period, and at one week and 6 weeks after discontinuation. Mean blood pressure was calculated as: (SBP+2×DBP)/3.

Brachial artery endothelium-dependent and -independent function was measured using ultrasound, at baseline, after 8 weeks of BH4 treatment, and 1 week after discontinuation of therapy. The technique was carried out as described in Prasad et al., Circulation, 2000. 101(20): p. 2349-54. Briefly, using an 11 MHz high resolution ultrasound transducer (Acuson Inc) in a temperature-controlled room, brachial diameter was measured above the antecubital fossa in the non-dominant arm. Flow-mediated vasodilation (FMD) was measured by inflating a BP cuff on the forearm to >200 mm Hg for 5 minutes, deflating rapidly, and measuring brachial diameter at 1 minute after onset of hyperemia. For all measurements three diameters were measured on three separate end-diastolic frames and averaged. To obtain the maximal brachial artery dilation that represents FMD the following equation was used: (average diameter with hyperemia−average baseline diameter)÷average baseline diameter X 100. To measure the endothelium-independent vasodilator response, subjects were given sublingual nitroglycerin 0.4 mg and the brachial diameter was measured as above after 5 minutes. To obtain endothelium-independent vasodilation the following equation was used: (average diameter post nitroglycerin-average baseline diameter)÷average baseline diameter X 100.

For both study 1 and study 2, the primary end point after statistical analysis was a reduction of systolic, mean and diastolic BP during BH4 therapy. Secondary end points included improvement of endothelial function as FMD. Linear mixed effects models for repeated measures data were used to analyze change in BP from baseline to end of treatment period and differences in change between 100 mg and 200 mg dose groups. A quadratic term for week was included in the model to account for non-linear trends over the treatment period. Dose, week and week squared were the main effect terms in the model; dose by week and dose by week squared were the interaction terms in the model. Significant interaction terms indicate a difference in the pattern of change between the two dose groups. FMD data was analyzed using the two-tailed student t-test. All results are expressed as mean±SD, and p values <0.05 are considered statistically significant.

The results for Study 1 showed that, in the combined group, there was a significant reduction in systolic (p=0.005 quadratic trend) and mean arterial BP (p=0.01) with BH4. Figure A displays the blood pressure response. Values are average standard error of mean. SBP=systolic blood pressure, MBP=mean blood pressure, DBP=diastolic blood pressure; p values were determined by ANOVA.

The results showed that systolic BP was lower by a mean of 13±9 mmHg after 3 weeks (p=0.004) and 15±15 mmHg (p=0.04) after 5 weeks and this reduction persisted for the 8 weeks of treatment. BP returned toward pre-treatment levels 6 weeks after discontinuation of therapy (p=ns, compared to baseline). There was no significant change in heart rate with BH4 and the change in diastolic BP did not reach statistical significance. There were also no statistically significant differences between the 5 mg/kg and 10 mg/kg doses of BH4.

Flow-mediated vasodilation of the brachial artery increased from a mean of 3.4±1% to 8.2±3.4% after 8 weeks of BH4 (p=0.05, n=6) and returned to baseline levels 6 weeks after termination of therapy (3.7±1.3%, p=ns compared to baseline). Nitroglycerin-mediated vasodilation remained unchanged (11.9±3% before and 16.1±5% after BH4, p=0.1).

Study 2: Investigation of Dose-Response of oral BH4:

Of the 20 subjects who qualified for participation, 4 were excluded during the run-in phase because of inability to return for follow-up appointments. There were 6 male subjects, mean age 59.5±8.3 years. Eight subjects received 200 mg b.i.d (twice daily) of oral BH4 and 8 received 100 mg b.i.d. of oral BH4. There were no significant differences in baseline characteristics between the two groups. Because BH4 was compounded with vitamin C in a 1:1 ratio, all subjects received vitamin C, at their assigned BH4 dose, twice a day during the first 2-week period. After this 2-week run-in period, subjects received BH4 only at their assigned dosage for the next 4 weeks. Weekly heart rate and BP measurements were made during the run-in period and treatment period. Mean blood pressure was calculated as: (SBP+2×DBP)/3. BH4 was then stopped and follow-up was performed at 1 and 4 weeks after discontinuation where BP and heart rate measurements were made. Routine chemistries and LFT's were drawn prior to initiation of medication, at the end of BH4 therapy and one month after discontinuation of BH4.

In study 2, brachial artery endothelium-dependent and independent function was measured, as described above, at the end of the 2-week vitamin C run-in period, at the end of 4 weeks of oral BH4 therapy, and 4 weeks after discontinuation of BH4.

There was no significant change in heart rate or BP during the run-in phase with vitamin C in either group; systolic, diastolic and mean arterial BP in the 16 subjects was 152±11, 84±13, 106±10 mmHg before, and 148±19, 86±11, and 106±12 mmHg, respectively, after 2 weeks of vitamin C (all p=ns).

Subjects given 200 mg b.i.d of oral BH4 had a significant decrease in systolic (p=0.03), and mean BP (p=0.04 by linear trend analysis). Figure B depicts the blood pressure response in the group treated with 200 mg BH4 b.i.d. Values are average standard error of mean. SBP=systolic blood pressure, MBP=mean blood pressure, DBP=diastolic blood pressure; p values were determined by ANOVA. The decrease in diastolic BP did not reach statistical significance (p=0.08). Mean BP was significantly lower after one week (p=0.02). There was a further significant reduction in BP during the subsequent weeks reaching a nadir after 3 weeks when systolic BP was a mean 16 mmHg lower; p=0.04 (Figure B). A week after termination of therapy, BP remained lower, but 4 weeks after discontinuation of BH4, BP rose and returned to baseline levels.

There was a significant improvement in FMD with 200 mg b.i.d. of BH4 (n=7). FMD improved from 3.7±3% at start of BH4 therapy to 7.1±4.9% after 4 weeks of therapy (p=0.016). One month after discontinuation of BH4 therapy, FMD returned to baseline levels (3.2±1.1%, p=0.6 compared to baseline).

No statistically significant change in BP was observed in subjects given 100 mg b.i.d. of BH4. Figure C depicts the blood pressure response in the group treated with 100 mg BH4 b.i.d. Values are average±standard error of mean. SBP=systolic blood pressure, MBP=mean blood pressure, DBP=diastolic blood pressure. There was no significant alteration in heart rate with BH4 in either group treated with 100 mg or 200 mg b.i.d.

There was no significant change in FMD in subjects given 100 mg b.i.d. of BH4 (5.3±2.5% vs 6.2±3.5%; p=0.55). There was no significant change in nitroglycerin-mediated, endothelium-independent vasodilation during the study in either group.

There were no significant adverse events during either study. The results demonstrate that long-term treatment with oral BH4 is effective in lowering arterial BP in subjects with either poorly controlled or newly diagnosed hypertension. This effect was observed at a dose of 200 mg b.i.d. or higher and was free of any changes in heart rate. Subjects had significant improvement in endothelial dysfunction after BH4 therapy at the doses that also resulted in reduction of BP. Reduction in BP was evident within 1 week of therapy and was sustained for up to 8 weeks of continuous therapy without tachyphylaxis. Blood pressure remained lower for at least a week after discontinuation of therapy, but returned toward baseline after 4 weeks. Oral BH4 appeared to be well tolerated without any serious adverse effects.

Further Clinical Studies

A Phase 2, multicenter, randomized, double blind, placebo-controlled, parallel study was initiated to evaluate the effects of 10 mg/kg/day 6R BH4, administered twice a day (b.i.d.) for 8 weeks, on blood pressure (BP) in subjects with poorly controlled systemic hypertension. A number of parameters are assessed, including arterial systolic blood pressure (SBP), arterial diastolic blood pressure (DBP), endothelial nitric oxide synthetase (eNOS) activity and endothelial dysfunction. In patients with both type 2 diabetes and poorly controlled hypertension, effect on insulin sensitivity is also assessed. Antihypertensive therapy and, where applicable, diabetes therapy remains unchanged throughout the study.

Criteria for inclusion in the study include a history of documented essential hypertension (BP of at least 140 mm Hg systolic and/or 90 mm Hg diastolic measured on 2 separate occasions) that is poorly controlled despite use of at least two conventional antihypertensive agents with different mechanisms of action taken concurrently and consistently for at least 3 months before randomization, a mean SBP and mean DBP during the initial two-week screening period within the following ranges: Mean SBP of at least 135 but not more than 160 mm Hg; Mean DBP of at least 85 but not more than 110 mm Hg. In addition, diabetic subjects must have a documented history of type 2 diabetes that has been treated using the same therapy for at least 3 months.

Criteria for exclusion from the study include: planned or potential pregnancy; previous treatment with any formulation of BH4; known allergy or hypersensitivity to any excipient of 6R BH4; known secondary cause for hypertension; a concurrent disease or condition that would interfere with study participation or safety such as bleeding disorders, history of syncope or vertigo, severe gastrointestinal reflux disease (GERD), symptomatic coronary or peripheral vascular disease, arrhythmia, organ transplant, organ failure, or type 1 diabetes mellitus; any severe co-morbid condition that would limit life expectancy to less than 6 months; serum creatinine >2.0 mg/dL or hepatic enzyme levels more than 2 times the upper limit of normal; concomitant treatment with (a) any drug known to inhibit folate metabolism (e.g., methotrexate), (b) levodopa or (c) any phosphodiesterase (PDE) 5 inhibitor (e.g., Viagra®, Clalis®, Levitra®, or Revatio™) or any PDE 3 inhibitor (e.g., cilostazol, milrinone, or vesnarinone).

Subjects that meet the inclusion criteria and are not excluded by the exclusion criteria receive either 6R BH4 or placebo for an 8 week treatment period and will have follow-up visits on Weeks 9 and 12 (i.e., 1 week and 4-week follow up). Trough BP and heart rate (HR) are measured weekly during the treatment period, and BP and HR are measured at both follow-up visits. The method for obtaining BP is standardized across all study centers and timing of BP measurements is standardized for each subject. Blood and urine samples for routine clinical laboratory tests and for biomarkers are collected at Weeks 0, 4, 8, and 12. Blood for fasting insulin and glucose levels and Hgb A1C is collected at Weeks 0, 8, and 12 for the diabetic cohort. ECGs are evaluated at Weeks 8 and 12.

At each visit, vital signs are recorded: SBP and DBP measured in mm Hg, heart rate (HR) in beats per minute and respiratory rate (RR) in breaths per minute. Physical examination includes assessment of weight, general appearance, neck, thorax/lungs, heart sounds, abdomen, and lower extremities.

During the two-week screening period, BP is measured 3 times (3 repetitions during a 10-minute period) at each of 3 visits. Mean SBP and DBP values are calculated by using these nine (9) measurements to determine whether the BP meets the inclusion criterion. After randomization, BP is again measured three times (Week 0 visit), and the mean of these three values is the baseline value.

During the treatment period beginning at Week 1, weekly trough BP measurements are obtained before the morning dose of study drug. BP should be taken at approximately the same time of day (within about 90 minutes).

A standard 12-lead electrocardiogram is recorded and collects the following measurements: heart rate, rhythm, interval measurements (i.e., PR, QRS, QT, QTc), and axis. A fully automatic, ambulatory BP monitoring (ABPM) apparatus is applied for 24 hours at treatment Weeks 0 and 8. The ABPM measures and records systolic and diastolic BP; multiple BP measurements can be plotted to represent the BP profile. Blood (plasma) and urine samples are collected to assess standard biomarkers of endothelial dysfunction and oxidative stress. Some subjects also undergo additional measurements of FMD, systemic vascular resistance, and arterial compliance using ultrasonography.

Clinical laboratory assessments on the blood and urine samples include the following:

| | |
|---|---|
| Hematology: | white blood cell count with differential, red blood cell |
| | count, platelet count, hemoglobin, and hematocrit |
| Serum chemistry: | albumin, alkaline phosphatase, ALT (SGPT), AST |
| | (SGOT), total bilirubin, BUN, calcium, chloride, total |
| | cholesterol, creatinine, GGT, globulin, glucose, LDH, |
| | phosphorous, potassium, total protein, sodium, and uric |
| | acid. |
| Fasting (4-6 h) serum lipids: | triglycerides, total cholesterol, low-density lipoprotein |
| | (LDL), and high-density lipoprotein (HDL) |
| Additional serum chemistry in | fasting glucose and insulin, and Hgb A₁C |
| subjects with type 2 diabetes: | |
| Urinalysis: | Routine: appearance, color, pH, specific gravity, ketones, |
| | protein, glucose, bilirubin, nitrite, urobilinogen, and |
| | microscopic examination |
| | First morning void sample collected for standard |
| | biomarkers (below): microalbuminuria |
| Biomarkers of endothelial | nitrates/nitrites, cGMP, and isoprostanes |
| dysfunction and oxidative stress: | |
| Standard biomarkers (plasma and | |
| urine): | |

Insulin sensitivity is evaluated in subjects with type 2 diabetes mellitus by using body mass index (BMI) and fasting serum insulin and glucose level results. HgbA1C is also measured.

Upon completion of the study, the effect of 10 mg/kg/day treatment with oral BH4 on blood pressure, endothelial function and insulin sensitivity is assessed. The invention contemplates that statistically significant reduction in either systolic and/or diastolic blood pressure is observed. Measures of endothelial function and/or insulin sensitivity may also improve after treatment with BH4. The effect of additional doses within the range of 2 mg/kg to 10 mg/kg per day is evaluated in further studies.

A Phase 2, Randomized, Open-Label, 2-Treatment, 2-Sequence, 2-Period Crossover, Pharmacokinetic (PK) Study to Compare the Plasma Concentrations of BH4 in Subjects With Endothelial Dysfunction Following 14 Days of Treatment by Each of 2 Regimens: 6R-BH4 with Vitamin C and 6R-BH4 Alone

### Objectives

The primary objective of the study is to compare plasma BH4 concentrations at the end of each of the 2 treatment regimens in subjects with endothelial dysfunction. The secondary objectives of the study are: to evaluate the safety of the 2 treatment regimens; to compare the 2 treatment regimens in terms of plasma concentration of total biopterins, BH2, and B; and the ratios BH4:BH2, BH4:BH2+B, and BH4:B; and to evaluate the efficacy of the 2 treatment regimens in improving endothelial function and in lowering blood pressure BP.

### Study Rationale

Clinical studies have shown that 6R-BH4 raises total biopterin concentration in plasma, but the relative increase in BH4 concentration versus the increase in the partially oxidized form, dihydrobiopterin (BH2), is unknown. Biopterin (B) is the completely oxidized form, and the relative increase in levels of BH4 versus the increase in B is also unknown when 6R-BH4 is given exogenously. Nonclinical data suggest that vitamin C may protect BH4 from oxidation, maintaining higher concentrations of BH4 and a higher BH4 to BH2 ratio (BH4:BH2). BH4 activates endothelial nitric oxide synthetase (eNOS), and BH2 is an eNOS uncoupler; the increase in the plasma BH4 concentration and/or the BH4:BH2 ratio may enhance eNOS activity and endothelial function. This study is designed to determine whether administration of 6R-BH4+vitamin C, compared with 6R-BH4 alone, will result in a higher plasma BH4 concentration, BH4:BH2, or both, in subjects with endothelial dysfunction. Study results will also be used to assess whether vitamin C enhances the expected BH4 actions of improving endothelial function and reducing blood pressure.

### Study Design and Plan:

This is a Phase 2, randomized, open-label, 2-treatment, 2-sequence, 2-period crossover, pharmacokinetic (PK) study to compare the plasma concentrations of BH4 in subjects with endothelial dysfunction following 14 days of treatment by each of 2 regimens: 6R-BH4+vitamin C and 6R-BH4 alone. Each subject receives each regimen; the 2 treatment groups vary only in the sequence of the 2 regimens.

A total of 40 subjects (20 per treatment group) are planned to complete this study. Some key criteria for including a subject in the study is a history of cardiovascular disease or cardiovascular risk factors, e.g., stable and well-controlled Type 2 diabetes, peripheral arterial disease, obesity, smoking, hypercholesterolemia, and endothelial dysfunction, documented at screening by an abnormal PAT of ≦1.70.

Study drug treatments include 6R-BH4 tablets, each containing 100 mg sapropterin dihydrochloride and vitamin C tablets or caplets, each containing 500 mg vitamin C All subjects receive treatment by 2 regimens as follows: Regimen 1-6R-BH4 5 mg/kg BID administered as whole tablets orally within 1 hour after morning and evening meals for 13 days and the last dose within 1 hour after a morning meal on Day 14 or Day 28; and Regimen 2-6R-BH4 5 mg/kg+500 mg vitamin C BID administered as whole tablets orally within 1 hour after morning and evening meals for 13 days and the last dose within 1 hour after a morning meal on Day 14 or Day 28.

The only difference between treatment groups is the sequence in which the regimens are administered. The duration of treatment is 28 days.

### Criteria for Evaluation

The primary efficacy variable of this study is the plasma BH4 concentration (area under the curve [AUC_{0-12 hr}]) at the end of each regimen in subjects with endothelial dysfunction. Secondary efficacy endpoints of interest, listed in order of clinical importance, are: plasma concentration of total biopterins, BH2, and B; and the ratios BH4:BH2, BH4:BH2+B, and BH4:B, calculated using AUC_{0-12 hr} at the end of each regimen; endothelial function, measured by peripheral arterial tonometry (PAT); mean daytime systolic blood pressure (SBP) and diastolic (DBP), measured by ambulatory blood pressure monitoring (ABPM); and plasma concentrations of biomarkers of endothelial function, oxidative stress, and inflammation.

Safety variables are incidence of adverse events (AEs), including serious AEs (SAEs), throughout the study; and changes from baseline in the following at the end of each regimen: ECGs, laboratory test results, and vital signs.

A Phase 2, Randomized, Double-Blind, Placebo-Controlled, Dose-Escalation, Parallel Study of Oral 6R-BH4 in Subjects with Isolated Systolic Hypertension (ISH) and Endothelial Dysfunction

### Study Rationale

Endothelial function, which underlies the ability of arteries to dilate appropriately in response to stimulus, is largely controlled by eNOS activity and the production of NO. Because the ability of eNOS to produce NO requires tetrahydrobiopterin (BH4) as a cofactor, an age-associated secondary BH4 deficiency may contribute to impaired eNOS activity resulting in decreased NO production and increased production of reactive oxygen species such as superoxide. This, in turn, is thought to contribute to endothelial dysfunction and arterial stiffness, and may contribute to Isolated Systolic Hypertension (ISH) in older adults. This study is designed to test the hypothesis that administration of oral 6R-BH4, a synthetic form of BH4 administered alone or concomitantly with antihypertension medications may enhance endothelial function, thus helping reduce SBP and ISH in this patient population.

### Study Design Plan

This Phase 2, randomized, double-blind, placebo-controlled, dose-escalation, parallel study is designed to assess the safety and efficacy of twice-daily oral dosing of 6R-BH4 to improve endothelial function, reduce SBP, and reduce arterial stiffness in subjects with ISH and endothelial dysfunction.

Any subject who is receiving therapy for hypertension is required to have been on a stable regimen for at least 30 days before the Screening Visit (Day—21). Upon entering the study, the subject starts a 3-week single-blind Run-in Period during which all subjects receive placebo BID, along with their currently prescribed antihypertension medications. The subject returns to the clinic for assessments as described in Table 1.

On Day 1, a subject who meets randomization criteria is randomized in a 1:1 ratio (6R-BH4:placebo) for the 8-week double-blind Dose-escalation Period: 4 weeks at 5 mg/kg BID or dosage-equivalent placebo; then 4 more weeks at 10 mg/kg BID or dosage-equivalent placebo, to be administered along with their currently prescribed antihypertension medications. During clinic visits, assessments of primary and secondary efficacy (endothelial function, BP, and arterial stiffness) are performed. Blood and urine samples are collected and diastolic function is measured for assessment of exploratory efficacy variables. Safety monitoring includes AE assessments, vital signs, electrocardiogram (ECG), and standard clinical laboratory tests.

By comparing values measured at different timepoints, the study is expected to provide insight regarding the ability of 6R-BH4, administered along with their currently prescribed antihypertension medications, to improve endothelial function, reduce SBP, and reduce arterial stiffness in patients with ISH and endothelial dysfunction. Additional measurements are expected to provide insight into the effects of 6R-BH4 on biochemical aspects of endothelial dysfunction, oxidative stress, and inflammation, and on diastolic function. Because endothelial dysfunction, ISH, and increased arterial stiffness are associated with increased cardiovascular risk, a novel treatment that targets these conditions may ultimately help to reduce this risk.

### Objectives

The primary objective of the study is to evaluate the efficacy of oral 6R-BH4 versus placebo to improve endothelial function, measured by flow-mediated dilation (FMD), in subjects with ISH and endothelial dysfunction after the 8-week Dose-escalation Period. The secondary objectives of the study are to evaluate the efficacy of oral 6R-BH4 versus dosage-equivalent placebo in subjects with ISH and endothelial dysfunction: to improve endothelial function, measured by FMD, after 4 weeks of treatment with 5 mg/kg BID; to reduce SBP, measured by ambulatory blood pressure monitoring (ABPM), after 4 weeks of treatment with 5 mg/kg BID; to reduce SBP, measured by ABPM, after the 8-week Dose-escalation Period; to reduce arterial stiffness after 4 weeks of treatment with 5 mg/kg BID; and to reduce arterial stiffness after the 8-week Dose-escalation Period. The exploratory objectives are to assess changes from baseline in levels in the blood and urine of biomarkers of endothelial function, oxidative stress and inflammation, and changes in diastolic function after 4 weeks at 5 mg/kg BID and after the 8-week Dose-escalation Period.

Approximately 60 subjects will be randomized with the expectation that 40 subjects will complete the study. A key criteria in including a subject in the study is ISH with the following mean seated BP: SBP≧145 and <180 mm Hg, diastolic blood pressure (DBP)<90 mm Hg.

During the double-blind Dose-escalation Period, subjects randomized to the 6R-BH4 group receive 6R-BH4 in coated tablets that each contain 100 mg of sapropterin dihydrochloride. The dosage is 5 mg/kg BID for the first 4 weeks. It is increased to 10 mg/kg BID for the following 4 weeks. Subjects take 2 doses each day: the first within 1 hour after the morning meal and the second within 1 hour after the last (evening) meal. The tablets are swallowed whole. The duration of treatment is 8 weeks (57 days).

During the single-blind Run-in Period, all subjects receive placebo tablets, indistinguishable from active tablets, at the sapropterin dihydrochloride dosage equivalent of 5 mg/kg BID. Subjects take 2 doses of placebo each day: the first within 1 hour after the morning meal and the second within 1 hour after the last (evening) meal. The tablets are swallowed whole.

During the Dose-escalation Period, subjects randomized to the placebo group receive placebo tablets administered in the same manner as 6R-BH4.

### Criteria for Evaluation

The primary efficacy variable is change in endothelial function measured by FMD, from baseline to Day 57 (Week 8). Secondary efficacy variables are: change in endothelial function, measured by FMD, from baseline to Day 29 (Week 4); change in mean daytime SBP, measured by ABPM, from baseline to Day 29 (Week 4); change in mean daytime SBP, measured by ABPM, from baseline to Day 57 (Week 8); change in arterial stiffness, measured noninvasively as augmentation; index (AI), pulse wave velocity (PWV), estimated central arterial pressure, and pulse pressure (PP), from baseline to Day 29 (Week 4); and change in arterial stiffness, measured noninvasively as AI, PWV, estimated central arterial pressure, and PP, from baseline to Day 57 (Week 8). Exploratory efficacy variables are changes from baseline to Day 29 (Week 4), and from baseline to Day 57 (Week 8) in: plasma cyclic guanosine monophosphate (cGMP); urine 8-isoprostanes; multianalyte profile (MAP) results; and diastolic function.

Safety criteria include incidence of treatment-emergent AEs throughout the study; changes in vital signs from baseline to scheduled timepoints; changes in laboratory test results from baseline to scheduled timepoints; and changes in ECG from baseline to scheduled timepoints.

### EXAMPLE 5

### Effect of BH4 in Combination with PDE5 Inhibitor in vivo

This study evaluated the cardiovascular effects of high doses of 6R-BH4 in combination with the PDE5 inhibitor sildenafil citrate in vivo. Eight male research nonnaïve and naïve purebred beagles were dosed in a double Latin square crossover design as described in the following table. All animals were dosed by oral gavage. Test articles were administered at a dose volume of 5 mL/kg (for a total volume of 10 mL/kg for the combination dose); control animals were dosed at a dose volume of 10 mL/kg. 6R-BH4 (sapropterin dihydrochloride) provided as 300 mg tablets (100 mg active 6R-BH4/tablet) stored at room temperature with desiccant.

| | | | | Dose Period |
|---|---|---|---|---|
| | Dose Period 1 | Dose Period 2 | Dose Period 3 | 4 |
| Animal | Day 1 | Day 4 | Day 8 | Day 11 |
| No. 1 | 6R-BH4^{a} | 6R-BH4 + | Control^{c} | Sildenafil^{d} |
| | | Sildenafil^{b} | | |
| No. 2 | 6R-BH4 + | Sildenafil | 6R-BH4 | Control |
| | Sildenafil | | | |
| No. 3 | Control | 6R-BH4 | Sildenafil | 6R-BH4 + |
| | | | | Sildenafil |
| No. 4 | Sildenafil | Control | 6R-BH4 + | 6R-BH4 |
| | | | Sildenafil | |
| No. 5 | Control | 6R-BH4 + | Sildenafil | 6R-BH4 |
| | | Sildenafil | | |
| No. 6 | 6R-BH4 + | 6R-BH4 | Control | Sildenafil |
| | Sildenafil | | | |
| No. 7 | Sildenafil | Control | 6R-BH4 | 6R-BH4 + |
| | | | | Sildenafil |
| No. 8 | 6R-BH4 | Sildenafil | 6R-BH4 + | Control |
| | | | Sildenafil | |

^{a}Animals given 6R-BH4 received 100 mg/kg at a dose volume of 5 mL/kg.
^{b}Animals given the combination treatment received 6R-BH4 (100 mg/kg; dose volume of 5 mL/kg) followed by a dose of sildenafil citrate (30 mg/kg; dose volume of 5 mL/kg) for a total volume of 10 mL/kg.
^{c}Animals in the control group received reverse osmosis water at a dose volume of 10 mL/kg.
^{d}Animals given sildenafil citrate received 30 mg/kg at a dose volume of 5 mL/kg.

At initiation of treatment, the animals were approximately 7 to 10 months old, and their body weights prior to dosing ranged from 7.8 to 12.0 kg.

At least 2 weeks prior to initiation of treatment, animals were fasted overnight, anesthetized, and an electrocardiogram (ECG) and blood pressure transmitter were implanted into the abdomen and sutured to the abdominal wall. The blood pressure catheter was placed in an artery and advanced to the abdominal aorta. Animals were checked twice daily (a.m. and p.m.) for mortality, abnormalities, and signs of pain and distress. Additional findings were recorded as observed. All animals survived to scheduled termination of the study.

Detailed observations were taken three times during the predose phase (including the day before dosing) and on Days 3 and 10 of the dosing phase. For each dosing day, ECG and pressure measurements were recorded for at least 90 minutes prior to dosing, continuously for at least 8 hours after dosing, and then for one period of at least 15 minutes in duration each hour through at least 24 hours postdose. Blood pressure measurements included systolic, diastolic, and mean arterial pressures and pulse pressure (systolic-diastolic). For each dosing day, blood pressure assessments were taken predose and approximately 2, 4, 8, 12, and 24 hours postdose.

Quantitative evaluation of ECG measurements, including RR interval, QT, and rate-corrected QT (QTc, using Fridericia's method), were done.

Positive inotropic effects (+dP/dtₘₐₓ) and heart rate were calculated from a left ventricular pressure signal predose and approximately 2, 4, 8, 12, and 24 hours postdose.

Assessment of cardiovascular effects and toxicity was based on mortality, clinical signs, body weights, abdominal temperature measurements, cardiovascular parameters including electrocardiographic analysis and hemodynamic data (systolic, diastolic, and mean arterial pressures), inotropic state (+dP/dtₘₐₓ), and heart rate. Mean systolic pressure for the various treatments over the 24 hour period after dosing is displayed in Figure D. Mean diastolic pressure for the various treatments over the 24 hour period after dosing is displayed in Figure E. Mean arterial pressure for the various treatments over the 24 hour period after dosing is displayed in Figure F. Mean arterial pulse pressure for the various treatments over the 24 hour period after dosing is displayed in Figure G. Mean (+dP/dtₘₐₓ) is displayed in Figure H. Mean heart rate is displayed in FIG. 1.

The QT interval measured on ECG was decreased at 2 and 4 hours postdose in animals given sildenafil citrate alone or in combination with 6R-BH4. The QT interval was not different between sildenafil citrate alone and in combination with 6R-BH4. No QT changes from controls were seen in animals given 6R-BH4 alone. Therefore, the QT changes were attributed to sildenafil citrate administration alone. No changes in heart rate corrected QT interval (QTc) were observed. The fact that the QTc normalized the QT interval data indicates that the decrease in QT interval was the result of increased heart rate (decreased RR interval) seen after sildenafil citrate administration. Two animals exhibited paroxysmal ventricular tachycardia and other animals exhibited isolated transient arrhythmias both before and after administration of test article; the arrhythmias were attributed to the implanted catheter in the left ventricle for pressure measurements rather than a result of the test articles.

No treatment-related systolic pressure, mean arterial pressure, or +dP/dtₘₐₓ changes were observed. Diastolic pressure was significantly decreased in animals given the combination dose or sildenafil citrate alone when compared with controls over all time points. This effect was not observed after administration of 6R-BH4 alone. No significant differences were seen between the effect of sildenafil citrate alone and in combination with 6R-BH4, suggesting this was primarily an effect of sildenafil citrate. The administration of 6R-BH4 along with sildenafil citrate did not augment or inhibit this effect.

Heart rate was significantly increased in animals given either sildenafil citrate alone or in combination with 6R-BH4 over all time points. No significant differences were noted between sildenafil citrate alone and sildenafil citrate in combination with 6R-BH4, suggesting this was primarily an effect of sildenafil citrate; 6R-BH4 did not augment or attenuate this effect.

Arterial pulse pressure was significantly increased in animals given sildenafil citrate alone at 2, 4, and 8 hours postdose. Arterial pulse pressure was significantly increased 2 and 4 hours after administration of sildenafil citrate and in combination with 6R-BH4. These changes were considered secondary to the decrease in diastolic pressure seen after these treatments.

The arterial pulse pressure was significantly decreased in animals given the combination of sildenafil citrate and 6R-BH4 when compared to administration of sildenafil citrate alone at the 2- and 8-hour time points, suggesting that 6R-BH4 may have an attenuating effect on the pulse pressure increase caused by sildenafil citrate.

Results of the study showed that sildenafil citrate and sildenafil citrate in combination with 6R-BH4 resulted in decreased diastolic pressure in dogs. This decrease in pressure was accompanied by an increase in heart rate and an increase in arterial pulse pressure. A decrease in blood pressure and an increase in heart rate are documented effects of sildenafil citrate in dogs. The increase in heart rate and pulse pressure was concluded to be secondary to the decrease in blood pressure. No effect of 6R-BH4 alone was noted on any of the cardiovascular parameters measured.

As noted, the results suggested an unexpected effect that the combination of PDE5 inhibitor with 6R-BH4 attenuated the observed sildenafil citrate-induced increase in pulse pressure in these dogs that would have been observed with sildenafil alone.

The addition of 100 mg of 6R-BH4/kg to 30 mg of sildenafil citrate/kg did not adversely affect the cardiovascular effects of sildenafil citrate in these dogs.

### EXAMPLE 6

### A Phase 2, Multicenter, Multinational, Randomized, Double-Blind, Placebo-Controlled, Parallel Study of the Effects of 6R-BH4 on Symptomatic Peripheral Arterial Disease (PAD)

### Objectives

The primary efficacy objective is to compare oral 6R-BH4 to placebo with respect to change from Baseline to Week 24 in peak walking time (PWT) in subjects with intermittent claudication (IC) caused by peripheral arterial disease (PAD).

The secondary efficacy objective is to compare oral 6R-BH4 to placebo with respect to change from Baseline to Week 24 in claudication onset time (COT) in subjects with IC caused by PAD. The tertiary efficacy objectives are to compare oral 6R-BH4 to placebo with respect to change from Baseline to Week 12 in PWT and claudication onset time (COT) in subjects with IC caused by PAD; to compare oral 6R-BH4 to placebo with respect to change from Baseline to Weeks 12 and 24 in the following in subjects with IC caused by PAD: functional status as measured by the Walking Impairment Questionnaire (WIQ) and Medical Outcomes Scale Short Form (MOS SF-36); mean systolic blood pressure (BP) (seated, rested); mean diastolic BP (seated, rested); ankle-brachial index (ABI) or toe-brachial index (TBI); International Index of Erectile Function (IIEF, short form) or the Female Sexual Function Index (FSFI), if available in subject's primary language; insulin sensitivity; biomarkers of endothelial nitric oxide synthase (eNOS) activity and oxidative stress as measured by cyclic guanosine monophosphate (cGMP) and 8-isoprostane, respectively; and endothelial function as measured by peripheral arterial tonometry (PAT). The safety objective is to assess the safety of oral dosing of 6R-BH4 in subjects with IC caused by PAD.

### Methodology

This Phase 2, multicenter, multinational, randomized, double-blind, placebo-controlled, parallel study is designed to assess the efficacy and safety of twice daily oral dosing of 6R-BH4 in subjects with IC caused by PAD.

Subjects who meet initial screening criteria will be monitored for up to 28 days to ensure that treadmill PWT on 2 consecutive tests meets the inclusion criteria and that dosages of permitted concomitant medications, including those for elevated cholesterol level, diabetes, or hypertension, are stable. The last treadmill test conducted at Screening, as well as the measurements of ABI or TBI, will serve as the baseline measurements for efficacy analyses.

Subjects will receive either 6R-BH4 or placebo. Approximately the first half (50%) of the subjects will receive either 6R-BH4 or placebo for a 24-week treatment period and will have a 1-week follow-up assessment by telephone on Week 25. The subsequent subjects will receive vitamin C with 6R-BH4 or placebo during the treatment period and will have a 1-week follow-up assessment by telephone on Week 25. Ankle brachial index (ABI) or toe brachial index (TBI) will be measured on Weeks 12 and 24. Trough treadmill tests will be administered on Weeks 12 and 24. Quality of life and International Index of Erectile Function (IIEF) or Female Sexual Function Index (FSFI) questionnaires will be administered prior to the treadmill test. Trough BP, heart rate (HR), and respiration rate will be measured at each visit during the treatment period. The method for administering treadmill tests and for obtaining BP will be standardized across all study centers, and timing of measurements will be standardized for each subject.

Blood and urine samples for routine clinical laboratory tests (including urine microalbumin level) and biomarkers of eNOS activity and oxidative stress will be collected at specified timepoints. Blood samples will also be analyzed for fasting lipids, insulin, and glucose, and for glycosylated hemoglobin A_{1c} (HbA_{1c}) levels at specified timepoints. A urine sample will be collected and stored at specified timepoints and may later be tested to monitor treatment compliance. ECGs will be evaluated at specified timepoints. Additional assessments for endothelial function will be measured at Baseline, Week 12 and Week 24 visits using PAT.

A sufficient number of subjects will be screened to ensure that approximately 210 subjects will be enrolled and approximately 174 subjects will complete the study. Individuals eligible to participate in this study include those who meet key criteria such as diagnosis of PAD secondary to atherosclerosis, with PAD documented at Screening by one of the following criteria: (a) Resting ABI <0.9 in at least one leg; (b) if resting ABI is 0.9-1.0, minimum post-exercise drop in ABI of at least 25% in at least one leg; (c) if resting ABI >1.3 (indicating non-compressible vessels), and vascular etiology documented by toe-brachial index (TBI)<0.7 in at least one leg. Other key criteria include PWT of at least 1 minute, but no more than 12 minutes on Gardner Treadmill Protocol and variation in PWT between two consecutive screening treadmill tests ≦25%.

The active treatment group will receive 6R-BH4 provided as tablets containing 100 mg of sapropterin dihydrochloride. This group will receive 800 mg/day of 6R-BH4, divided into two doses each day; 4 tablets in the morning within one hour after eating and 4 tablets within one hour after the last (evening) meal of the day. Tablets will be taken orally. When approximately 50% enrollment is reached, all subjects enrolled thereafter will also receive 1000 mg/day of vitamin C, given orally in two divided doses of 500 mg, with study drug. Subjects receiving vitamin C will continue to receive vitamin C until the completion of study. Subjects not receiving vitamin C will never receive vitamin C throughout the study. The duration of treatment is 24 weeks. Placebo tablets will be administered in the same manner as that of the investigational product. When approximately 50% enrollment is reached, all subjects enrolled thereafter will also receive 1000 mg/day of vitamin C, given orally in two divided doses of 500 mg, with placebo. Subjects receiving vitamin C will continue to receive vitamin C until the completion of study. Subjects not receiving vitamin C will never receive vitamin C throughout the study.

### Criteria for Evaluation:

The primary efficacy criteria is a mean change in PWT from Baseline to Week 24. The secondary efficacy criteria is a mean change in COT from Baseline to Week 24. The tertiary efficacy criteria are (1) a mean change from Baseline to Week 12 in PWT and COT and (2) a mean change from Baseline to Weeks 12 and 24 in functional status, as measured by the Walking Impairment Questionnaire (WIQ) and Medical Outcomes Scale Short Form (MOS SF-36), mean systolic BP (seated, rested), mean diastolic BP (seated, rested), ABI or TBI, IIEF (short form) or the FSFI, if available in subject's primary language, insulin sensitivity, biomarkers of eNOS activity and oxidative stress as measured by cGMP and 8-isoprostane, respectively, and noninvasive measurements of endothelial function using PAT at study sites selected for substudy of additional and exploratory measure of efficacy.

The safety criteria are based on the adverse event assessment, physical examination, vital signs, ECG, clinical laboratory tests (CBC, chemistry, urinalysis including urine microalbumin test), fasting serum lipids, thyroid stimulating hormone (TSH), T3, T4 and change in concomitant medications.

### EXAMPLE 7

### Safety and Efficacy of Tetrahydrobiopterin in Patients with Chronic Kidney Disease (CKD) and Albuminuria: An Open-Label Pilot Study

### Objectives

The primary objective is to evaluate the safety and efficacy of 6R-BH4 given alone or with vitamin C (6R-BH4+vitamin C) in reducing albuminuria (measured in a 24-hour urine collection as well as by albumin/creatinine ratio in spot early morning urine samples) in patients with mild to moderate chronic kidney disease (CKD) with estimated glomerular filtration rate (eGFR)≧40 ml/min/1.73 m². The secondary objective is to evaluate the effects of 6R-BH4 alone or with Vitamin C on eGFR, and blood pressure. The safety objective is to assess the safety of 400 mg BID oral dosing of 6R-BH4 with or without 500 mg BID vitamin C in subjects with mild to moderate CKD.

### Study Design and Plan

This Phase 2 study is designed to assess the efficacy and safety of twice daily oral dosing of 6R-BH4 in subjects with mild to moderate CKD. Subjects will receive 6R-BH4 for a 6-week treatment period and then will receive 6R-BH4 plus vitamin C for an additional 6 weeks. Patients will have a follow-up visit on Week 18. Spot early morning urine for albumin:creatinine ratio will be collected at Weeks −1, 0, 3, 6, 9, 12, and 16 and 24-hour urine collection for albumin and creatinine will be performed at Weeks 0, 6, 9, 12 and 18. Blood and urine samples for routine clinical laboratory tests will be collected at Weeks 0, 3, 6, 9, 12 and 16 and samples for special biomarkers at Week 0, 6 and 12 will be archived. ECGs will be evaluated at Weeks—1 and 12, and vital signs, including trough blood pressures, will be recorded at each visit.

### Hypothesis:

6R-BH4 alone or in conjunction with high dose vitamin C is hypothesized to improve the availability of NO in patients with CKD and improve endothelial function. This is postulated to have an effect on reducing albuminuria in patients with CKD and would have implications for stabilization of renal function as well as reduction in cardiovascular risk. In this pilot study, it is anticipated that treatment with BH4 will be associated with at least 15% (or greater) reduction in albuminuria (measured as albumin excretion in the 24-hour urine samples as well as by urine albumin:creatinine ratio in early morning spot urine) as a result of BH4 alone (after 6 weeks of dosing) or combined with vitamin C (after 12 weeks of dosing).

Thirty subjects are planned to complete the study. A key criteria for including a subject in the study is a diagnosis of clinically stable CKD (estimated Glomerular Filtration Rate (eGFR)≧40 ml/min/1.73 m² by the abbreviated (4-variable) MDRD equation and with a rate of decline of eGFR no greater than 1 ml/min/1.73 m² per month over the prior 3 months) with albuminuria (urine albumin excretion in the 24-hr urine sample of between 100-3000 mg).

Study drug treatments include 6R-BH4 tablets, each containing 100 mg sapropterin dihydrochloride, administered at 400 mg orally twice daily for 6 weeks, then 6R-BH4 tablets, each containing 100 mg sapropterin dihydrochloride, administered at 400 mg orally twice daily plus 500 mg orally twice daily vitamin C for 6 weeks. Subjects will take the first dose in the morning within one hour after eating and the second dose within one hour after the last (evening) meal of the day. The duration of treatment will be 12 weeks and duration of study will be 18 weeks.

### Criteria for Evaluation

The primary efficacy endpoints are 24-hour urinary albumin excretion and albumin/creatinine ratio in spot urine specimen. The secondary efficacy endpoints include estimated glomerular filtration rate (using the abbreviated MDRD equation) and trough seated, supine and standing blood pressures.

The safety criteria are based on general tolerability of the drug (symptoms such as dizziness, malaise, weakness, etc); effect on renal function as measured by eGFR; incidence of adverse effects and significant adverse effects throughout the study including hypotension as evidenced by orthostatic symptoms or by actual clinical hypotensive episodes; allergic reactions (cutaneous or other); gastrointestinal side effects such as nausea, vomiting, altered taste, etc.; changes in ECG from baseline to Week 12; changes in laboratory test results from baseline to Week 12; and changes in vital signs from baseline to Week 12.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating an endothelial dysfunction comprising administering to a human in need thereof (6R)-tetrahydrobiopterin (BH4) in a range of about 1 mg/kg to about 15 mg/kg per day to treat said endothelial dysfunction.

2. The method of claim 1, wherein the amount of BH4 administered is in a range of greater than 3 mg/kg to about 15 mg/kg per day.

3. The method of claim 1, wherein the amount of BH4 administered is up to about 10 mg/kg per day.

4. The method of claim 2, wherein the amount of BH4 administered is 10 mg/kg per day or less.

5. The method of claim 2, wherein the amount of BH4 administered is 10 mg/kg per day.

6. The method of claim 2, wherein the amount of BH4 administered is 5 mg/kg per day.

7. The method of claim 1, wherein the endothelial dysfunction is hypertension.

8. The method of claim 1, wherein the human suffers from diabetes mellitus type 2.

9. The method of claim 1, wherein the administering is performed orally.

10. The method of claim 1, wherein the BH4 is administered at least twice daily.

11. The method of claim 10, wherein the BH4 is administered only twice daily.

12. A method of treating an endothelial dysfunction comprising the step of administering to a human in need thereof (6R)-tetrahydrobiopterin (BH4) in an amount effective to treat said endothelial dysfunction and a therapeutic drug other than BH4.

13. The method of claim 12, wherein the therapeutic drug other than BH4 comprises a PDE 5 inhibitor.

14. The method of claim 13, wherein the PDE 5 inhibitor is selected from the group consisting of sildenafil, a pharmaceutically acceptable salt of sildenafil, a solvate of sildenafil, tadalafil, a pharmaceutically acceptable salt of tadalafil, a solvate of tadalafil, vardenafil, a pharmaceutically acceptable salt of vardenafil, a solvate of vardenafil, udenafil, a pharmaceutically acceptable salt of udenafil, a solvate of udenafil, and mixtures thereof.

15. The method of claim 13, wherein the PDE 5 inhibitor comprises sildenafil or a pharmaceutically acceptable salt or solvate thereof.

16. A method of treating a sickle cell disease comprising the step of administering to a human in need thereof (6R)-tetrahydrobiopterin (BH4) in an amount effective to treat said sickle cell disease.

17. The method of claim 16, wherein the effective amount is in a range of about 1 mg/kg per day to about 20 mg/kg per day.

18. The method of claim 17, wherein the effective amount is in a range of about 2 mg/kg per day to about 10 mg/kg per day.

19. A method of treating an intermittent claudication comprising the step of administering to a human in need thereof (6R)-tetrahydrobiopterin (BH4) in an amount effective to treat said intermittent claudication.

20. The method of claim 19, wherein the effective amount is in a range of about 1 mg/kg per day to about 20 mg/kg per day.

21. The method of claim 20, wherein the effective amount is in a range of about 2 mg/kg per day to about 10 mg/kg per day.

22. A method of treating a pulmonary arterial hypertension comprising the step of administering to a human in need thereof (6R)-tetrahydrobiopterin (BH4) in an amount effective to treat said pulmonary arterial hypertension.

23. The method of claim 22, wherein the effective amount is in a range of about 1 mg/kg per day to about 20 mg/kg per day.

24. The method of claim 23, wherein the effective amount is in a range of about 2 mg/kg per day to about 10 mg/kg per day.

25. A method of treating albuminuria associated with chronic kidney disease comprising the step of administering to a human in need thereof (6R)-tetrahydrobiopterin (BH4) in an amount effective to treat said albuminuria associated with chronic kidney disease.

26. The method of claim 25, wherein the effective amount is in a range of about 1 mg/kg per day to about 20 mg/kg per day.

27. The method of claim 26, wherein the effective amount is in a range of about 2 mg/kg per day to about 10 mg/kg per day.

28. A method of treating isolated systolic hypertension comprising the step of administering to a human in need thereof (6R)-tetrahydrobiopterin (BH4) in an amount effective to treat said isolated systolic hypertension.

29. The method of claim 28, wherein the effective amount is in a range of about 1 mg/kg per day to about 20 mg/kg per day.

30. The method of claim 29, wherein the effective amount is in a range of about 2 mg/kg per day to about 10 mg/kg per day.

31. A method of treating albuminuria associated with coronary artery disease comprising the step of administering to a human in need thereof (6R)-tetrahydrobiopterin (BH4) in an amount effective to treat said coronary artery disease.

32. The method of claim 31, wherein the effective amount is in a range of about 1 mg/kg per day to about 20 mg/kg per day.

33. The method of claim 32, wherein the effective amount is in a range of about 2 mg/kg per day to about 10 mg/kg per day.
